SEQUENCE LISTING (76 sequences)

SEQ ID NO: 1 - DNA, 2076 bp, Homo sapiens
  CDS (1)..(2076)
atgggcaccgtcagctccaggcggtcctggtggccgctgccactgctgctgctgctgctgctgctcctgggtcccgcgggcgcccgtgcgcaggaggacgaggacggcgactacgaggagctggtgctagccttgcgctccgaggaggacggcctggccgaagcacccgagcacggaaccacagccaccttccaccgctgcgccaaggatccgtggaggttgcctggcacctacgtggtggtgctgaaggaggagacccacctctcgcagtcagagcgcactgcccgccgcctgcaggcccaggctgcccgccggggatacctcaccaagatcctgcatgtcttccatggccttcttcctggcttcctggtgaagatgagtggcgacctgctggagctggccttgaagttgccccatgtcgactacatcgaggaggactcctctgtctttgcccagagcatcccgtggaacctggagcggattacccctccgcggtaccgggcggatgaataccagccccccgacggaggcagcctggtggaggtgtatctcctagacaccagcatacagagtgaccaccgggaaatcgagggcagggtcatggtcaccgacttcgagaatgtgcccgaggaggacgggacccgcttccacagacaggccagcaagtgtgacagtcatggcacccacctggcaggggtggtcagcggccgggatgccggcgtggccaagggtgccagcatgcgcagcctgcgcgtgctcaactgccaagggaagggcacggttagcggcaccctcataggcctggagtttattcggaaaagccagctggtccagcctgtggggccactggtggtgctgctgcccctggcgggtgggtacagccgcgtcctcaacgccgcctgccagcgcctggcgagggctggggtcgtgctggtcaccgctgccggcaacttccgggacgatgcctgcctctactccccagcctcagctcccgaggtcatcacagttggggccaccaatgcccaggaccagccggtgaccctggggactttggggaccaactttggccgctgtgtggacctctttgccccaggggaggacatcattggtgcctccagcgactgcagcacctgctttgtgtcacagagtgggacatcacaggctgctgcccacgtggctggcattgcagccatgatgctgtctgccgagccggagctcaccctggccgagttgaggcagagactgatccacttctctgccaaagatgtcatcaatgaggcctggttccctgaggaccagcgggtactgacccccaacctggtggccgccctgccccccagcacccatggggcaggttggcagctgttttgcaggactgtgtggtcagcacactcggggcctacacggatggccacagccatcgcccgctgcgccccagatgaggagctgctgagctgctccagtttctccaggagtgggaagcggcggggcgagcgcatggaggcccaagggggcaagctggtctgccgggcccacaacgcttttgggggtgagggtgtctacgccattgccaggtgctgcctgctaccccaggccaactgcagcgtccacacagctccaccagctgaggccagcatggggacccgtgtccactgccaccaacagggccacgtcctcacaggctgcagctcccactgggaggtggaggaccttggcacccacaagccgcctgtgctgaggccacgaggtcagcccaaccagtgcgtgggccacagggaggccagcatccacgcttcctgctgccatgccccaggtctggaatgcaaagtcaaggagcatggaatcccggcccctcaggggcaggtgaccgtggcctgcgaggagggctggaccctgactggctgcagcgccctccctgggacctcccacgtcctgggggcctacgccgtagacaacacgtgtgtagtcaggagccgggacgtcagcactacaggcagcaccagcgaagaggccgtgacagccgttgccatctgctgccggagccggcacctggcgcaggcctcccaggagctccag

SEQ ID NO: 2 - protein, 692 aa, Homo sapiens
MGTVSSRRSWWPLPLLLLLLLLLGPAGARAQEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAIARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQGQVTVACEEGWTLTGCSALPGTSHVLGAYAVDNTCVVRSRDVSTTGSTSEEAVTAVAICCRSRHLAQASQELQ

SEQ ID NO: 3 - DNA, 2076 bp, Macaca fascicularis
  CDS (1)..(2076)
atgggtaccgtcagctccaggcggtcctggtggcctctgccgctgccactgctgctgctcctgctcctgggtcccgctggcgcccgtgcgcaggaggacgaggacggcgactacgaggagctggtgctagccttgcgttccgaggaggacggcctggccgacgcacccgagcacggagccacagccaccttccaccgctgcgccaaggatccgtggaggttgcccggcacctacgtggtggtgctgaaggaggagacccaccgctcgcagtcagagcgcactgcccgccgcctgcaggcccaagctgcccgccggggatacctcaccaagatcctgcatgtcttccatcaccttcttcctggcttcctggtgaagatgagtggcgacctgctggagctggccctgaagttgccccatgtcgactacatcgaggaggactcctctgtcttcgcccagagcatcccatggaacctggagcgaattactcctgcacggtaccgggcggatgaataccagccccccaaaggaggcagcctggtggaggtgtatctcctagacaccagcatacagagtgaccaccgggaaatcgagggcagggtcatggtcaccgacttcgagagtgtgcccgaggaggacgggacccgcttccacagacaggccagcaagtgtgacagccatggcacccacctggcaggggtggtcagcggccgggatgccggcgtggccaagggcgccggcctgcgtagcctgcgcgtgctcaactgccaagggaagggcacggtcagcggcaccctcataggcctggagtttattcggaaaagccagctggtccagcccgtggggccactggttgtgctgctgcccctggcgggtgggtacagccgggtcttcaacgccgcctgccagcgcctggcgagggctggggtcgtgctggtcaccgctgccggcaacttccgggacgatgcctgcctctactctccagcctcggctcccgaggtcatcacagttggggccaccaatgcccaggaccagccggtgaccctggggactttggggaccaactttggccgctgtgtggacctctttgccccaggggaggacatcattggtgcctccagcgactgcagcacctgctttgtgtcacggagtgggacatcgcaggctgctgcccacgtggctggcattgcagccatgatgctgtctgccgagccggagctcactctggccgagttgaggcagagactgatccacttctctgccaaagatgtcatcaatgaggcctggttccctgaggaccagcgggtactgacccccaacctggtggccgccctgccccccagcacccacagggcaggttggcagctgttttgcaggactgtgtggtcagcacactcgggtcctacacggatggccacagccgtagcccgctgcgcccaggatgaggagctgctgagctgctccagtttctccaggagtgggaagcggcggggcgagcgcatcgaggcccaagggggcaagcgggtctgccgggcccacaacgcttttgggggtgagggtgtctacgccattgccaggtgctgcctgctaccccaggtcaactgcagcgtccacacagctccaccagctggggccagcatggggacccgtgtccactgccatcagcagggccacgtcctcacaggctgcagctcccactgggaggtggaggaccttggcacccacaagccgcctgtgctgaggccacgaggtcagcccaaccagtgtgtgggccacagggaggccagcatccacgcttcctgctgccatgccccaggtctggaatgcaaagtcagggagcatggaatcccggcccctcaggagcaggttatcgtggcctgtgaggacggctggaccctgaccggctgcagtgccctccctgggacctcccatgtcctgggggcctacgctgtagacaacacgtgtgtggtcaggagccgggacgtcagcaccacaggcagcaccagcgaagaagccgtggcagccgttgccatctgctgccggagccggcacctggtgcaggcctcccaggagctccag

SEQ ID NO: 4 - protein, 692 aa, Macaca fascicularis
MGTVSSRRSWWPLPLPLLLLLLLGPAGARAQEDEDGDYEELVLALRSEEDGLADAPEHGATATFHRCAKDPWRLPGTYVVVLKEETHRSQSERTARRLQAQAARRGYLTKILHVFHHLLPGFLVKMSGDLLELALKLPHVDYIEEDSSVFAQSIPWNLERITPARYRADEYQPPKGGSLVEVYLLDTSIQSDHREIEGRVMVTDFESVPEEDGTRFHRQASKCDSHGTHLAGVVSGRDAGVAKGAGLRSLRVLNCQGKGTVSGTLIGLEFIRKSQLVQPVGPLVVLLPLAGGYSRVFNAACQRLARAGVVLVTAAGNFRDDACLYSPASAPEVITVGATNAQDQPVTLGTLGTNFGRCVDLFAPGEDIIGASSDCSTCFVSRSGTSQAAAHVAGIAAMMLSAEPELTLAELRQRLIHFSAKDVINEAWFPEDQRVLTPNLVAALPPSTHRAGWQLFCRTVWSAHSGPTRMATAVARCAQDEELLSCSSFSRSGKRRGERIEAQGGKRVCRAHNAFGGEGVYAIARCCLLPQVNCSVHTAPPAGASMGTRVHCHQQGHVLTGCSSHWEVEDLGTHKPPVLRPRGQPNQCVGHREASIHASCCHAPGLECKVREHGIPAPQEQVIVACEDGWTLTGCSALPGTSHVLGAYAVDNTCVVRSRDVSTTGSTSEEAVAAVAICCRSRHLVQASQELQ

SEQ ID NO: 5 - protein, 326 aa, ARTIFICIAL SEQUENCE
  IgG2 heavy chain
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 6 - protein, 107 aa, ARTIFICIAL SEQUENCE [sequence omitted]

SEQ ID NO: 7 - protein, 105 aa, ARTIFICIAL SEQUENCE
  LAMBDA LIGHT CHAIN
QPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 8 - protein, 216 aa, ARTIFICIAL SEQUENCE
  Antibody Light Chain
QSVLTQPPSVSAAPGQKVTISCSGSSSNIGINYVSWYQLLPGTAPKLLIYDNNKRPLGIPDRFSGSKSGTSATLGITGLQTGDETDYYCGTWDSSLSAWVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 9 - protein, 216 aa, ARTIFICIAL SEQUENCE
  Antibody Light Chain
QSVLTQPPSVSAAPGQKVTISCSGSSSNIGINYVSWYQLLPGTAPKLLIYDNNKRPLGIPDRFSGSKSGTSVTLGITGLQTGDEADYFCGTWDSNLSAWVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 10 - protein, 212 aa, ARTIFICIAL SEQUENCE
  Antibody Light Chain
SYELTQPPSVSVSPGQTASITCSGDKLGDKYACWFQQKPGQSPVLVIYQDSKRPFGIPDRFSGSKSGNTATLTISGTQAMEEADYYCQAWDSSTAVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 11 - protein, 212 aa, ARTIFICIAL SEQUENCE
  Antibody Light Chain
SYELTQPPSVSVSPGQTISISCSGDKLGDKNACWYQQKPGQSPGLVIYQDSKRPLGIPARFSGSNSGNTATLTISGTQAMDEADYYCQAWDISTVVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 12 - protein, 451 aa, ARTIFICIAL SEQUENCE
  Antibody Heavy Chain
QVQLVQSGAEVKKPGASVKVSCKASGYTFSSYGISWVRQAPGQGLEWMGWISAYNGNTNYAQKFQGRVTMTTDTSTSTAYMELRSLRSDDTAVYYCAREVRLAARPDHYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 13 - protein, 451 aa, ARTIFICIAL SEQUENCE; identical to SEQ ID NO: 12
  Antibody Heavy Chain

SEQ ID NO: 14 - protein, 450 aa, ARTIFICIAL SEQUENCE
  Antibody Heavy Chain
QGQLVESGGGVVQPGRSLRLSCAASGFTFSSYGMHWVRQAPGKGLEWVAVIWHDGSNKYYADSVKGRFTISRDNSKNTLYLQMNSLRAEDTAVYYCARVVPDNWNQDYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 15 - protein, 446 aa, ARTIFICIAL SEQUENCE
  Antibody Heavy Chain
QVQLVESGGGVVQPGRSLRLSCAASGFTFSSYGMHWVRQAPGKGLEWVAAIWYDGSNKYYAESVKGRFTISRDNSKYTLYLQVNSLRAEDTAVYYCARRGGLAARPGGLDVWGQGTTVTVASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 16 - protein, 111 aa, ARTIFICIAL SEQUENCE
  Antibody Variable Light Chain
QSVLTQPPSVSAAPGQKVTISCSGSSSNIGINYVSWYQLLPGTAPKLLIYDNNKRPLGIPDRFSGSKSGTSATLGITGLQTGDETDYYCGTWDSSLSAWVFGGGTKLTVLG

SEQ ID NO: 17 - protein, 111 aa, ARTIFICIAL SEQUENCE
  Antibody Variable Light Chain
QSVLTQPPSVSAAPGQKVTISCSGSSSNIGINYVSWYQLLPGTAPKLLIYDNNKRPLGIPDRFSGSKSGTSVTLGITGLQTGDEADYFCGTWDSNLSAWVFGGGTKLTVLG

SEQ ID NO: 18 - protein, 107 aa, ARTIFICIAL SEQUENCE
  Antibody Variable Light Chain
SYELTQPPSVSVSPGQTASITCSGDKLGDKYACWFQQKPGQSPVLVIYQDSKRPFGIPDRFSGSKSGNTATLTISGTQAMEEADYYCQAWDSSTAVFGGGTKLTVLG

SEQ ID NO: 19 - protein, 107 aa, ARTIFICIAL SEQUENCE
  Antibody Variable Light Chain
SYELTQPPSVSVSPGQTISISCSGDKLGDKNACWYQQKPGQSPGLVIYQDSKRPLGIPARFSGSNSGNTATLTISGTQAMDEADYYCQAWDISTVVFGGGTKLTVLG

SEQ ID NO: 20 - protein, 125 aa, ARTIFICIAL SEQUENCE
  Antibody Variable Heavy Chain
QVQLVQSGAEVKKPGASVKVSCKASGYTFSSYGISWVRQAPGQGLEWMGWISAYNGNTNYAQKFQGRVTMTTDTSTSTAYMELRSLRSDDTAVYYCAREVRLAARPDHYYGMDVWGQGTTVTVSS

SEQ ID NO: 21 - protein, 125 aa, ARTIFICIAL SEQUENCE; identical to SEQ ID NO: 20
  Antibody Variable Heavy Chain

SEQ ID NO: 22 - protein, 124 aa, ARTIFICIAL SEQUENCE
  Antibody Variable Heavy Chain
QGQLVESGGGVVQPGRSLRLSCAASGFTFSSYGMHWVRQAPGKGLEWVAVIWHDGSNKYYADSVKGRFTISRDNSKNTLYLQMNSLRAEDTAVYYCARVVPDNWNQDYYGMDVWGQGTTVTVSS

SEQ ID NO: 23 - protein, 120 aa, ARTIFICIAL SEQUENCE
  Antibody Variable Heavy Chain
QVQLVESGGGVVQPGRSLRLSCAASGFTFSSYGMHWVRQAPGKGLEWVAAIWYDGSNKYYAESVKGRFTISRDNSKYTLYLQVNSLRAEDTAVYYCARRGGLAARPGGLDVWGQGTTVTV

SEQ ID NO: 24 - DNA, 333 bp, ARTIFICIAL SEQUENCE
  Antibody Variable Light Chain
cagtctgtgttgacgcagccgccctcagtgtctgcggccccaggacagaaggtcaccatctcctgctctggaagcagctccaacattggcattaattatgtatcctggtaccaactactcccaggaacagcccccaaactcctcatttatgacaataataagcgacccttagggattcctgaccgattctctggctccaagtctggcacgtcagccaccctgggcatcaccggactccagactggggacgagaccgattattactgtggaacatgggatagcagcctgagtgcttgggtgttcggcggagggaccaagctgaccgtcctaggt

SEQ ID NO: 25 - DNA, 333 bp, ARTIFICIAL SEQUENCE
  Antibody Variable Light Chain
cagtctgtgttgacgcagccgccctcagtgtctgcggccccaggacagaaggtcaccatctcctgctctggaagtagctccaacattgggattaattatgtatcctggtaccagctactcccaggaacagcccccaaactcctcatttatgacaataataagcgacccttagggattcctgaccgattctctggctccaagtctggcacgtcagtcaccctgggcatcaccggactccagactggggacgaggccgattatttctgcggaacatgggatagcaacctgagtgcttgggttttcggcggagggaccaagctgaccgtcctaggt

SEQ ID NO: 26 - DNA, 321 bp, ARTIFICIAL SEQUENCE
  Antibody Variable Light Chain
tcctatgagctgactcagccaccctcagtgtccgtgtccccaggacagacggccagcatcacctgctctggagataaattgggggataaatatgcttgctggtttcagcagaagccaggccagtcccctgtgctggtcatttatcaagatagcaagcggccctttgggatccctgaccgattctctggctccaagtctgggaacacagccactctgaccatcagcgggacccaggctatggaagaggctgactattactgtcaggcgtgggacagtagcactgcggtattcggcggagggaccaagctgaccgtcctaggt

SEQ ID NO: 27 - DNA, 321 bp, ARTIFICIAL SEQUENCE
  Antibody Variable Light Chain
tcctatgagctgactcagccaccctcagtgtccgtgtccccaggacagacaatctccatcagctgttctggagataaattgggggataaaaatgcttgctggtatcagcagaagccaggccagtcccctgggctggtcatctatcaggatagcaagcggcccttagggatccctgcgcgattctctggctccaactctgggaacacagccactctgaccatcagcgggacccaggctatggatgaggctgactattactgtcaggcgtgggacatcagcactgtggtattcggcggagggaccaagctgaccgtcctaggt

SEQ ID NO: 28 - DNA, 375 bp, ARTIFICIAL SEQUENCE
  Antibody Variable Heavy Chain
caggttcagctggtgcagtctggagctgaggtgaagaagcctggggcctcagtgaaggtctcctgcaaggcctctggttacacctttagcagttatggtatcagctgggtgcgacaggcccctggacaagggcttgagtggatgggatggatcagcgcttacaatggtaatacaaactatgcacagaagttccagggcagagtcaccatgaccacagacacatccacgagcacagcctacatggagctgaggagcctgagatctgacgacacggccgtgtattactgtgcgagagaagttcgtctggcagctcgtcctgaccattactacggtatggacgtctggggccaagggaccacggtcaccgtctcctca

SEQ ID NO: 29 - DNA, 375 bp, ARTIFICIAL SEQUENCE; identical to SEQ ID NO: 28
  Antibody Variable Heavy Chain

SEQ ID NO: 30 - DNA, 372 bp, ARTIFICIAL SEQUENCE
  Antibody Variable Heavy Chain
caggggcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagtcatatggcatgatggaagtaataaatactatgcagactccgtgaagggccgattcaccatctccagagacaattccaagaacacgctgtatctgcaaatgaacagcctgagagccgaggacacggctgtgtattactgtgcgagagtcgttcccgataactggaaccaggactactacggtatggacgtctggggccaagggaccacggtcaccgtctcctca

SEQ ID NO: 31 - DNA, 366 bp, ARTIFICIAL SEQUENCE
  Antibody Variable Heavy Chain
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagctatatggtatgatggaagtaataaatactatgcagagtccgtgaagggccgattcaccatctccagagacaattccaagtacacgctgtatctgcaagtgaacagcctgagagccgaggacacggctgtgtattactgtgcgaggagggggggtctggcagctcgtccgggcggtctggacgtctggggccaagggaccacggtcaccgtctcctca

SEQ ID NO: 32-33 - skipped (2 entries)

SEQ ID NO: 34 - protein, 5 aa, ARTIFICIAL SEQUENCE
  CDRH Sequence
SYGIS

SEQ ID NO: 35 - protein, 5 aa, ARTIFICIAL SEQUENCE
  CDRH Sequence
SYGMH

SEQ ID NO: 36 - protein, 17 aa, ARTIFICIAL SEQUENCE
  CDRH Sequence
WISAYNGNTNYAQKFQG

SEQ ID NO: 37 - protein, 17 aa, ARTIFICIAL SEQUENCE
  CDRH Sequence
VIWHDGSNKYYADSVKG

SEQ ID NO: 38 - protein, 16 aa, ARTIFICIAL SEQUENCE
  CDRH Sequence
EVRLAARPDHYYGMDV

SEQ ID NO: 39 - protein, 15 aa, ARTIFICIAL SEQUENCE
  CDRH Sequence
VVPDNWNQDYYGMDV

SEQ ID NO: 40 - protein, 5 aa, ARTIFICIAL SEQUENCE
  CDRH Sequence
SYGMH

SEQ ID NO: 41 - protein, 17 aa, ARTIFICIAL SEQUENCE
  CDRH Sequence
AIWYDGSNKYYAESVKG

SEQ ID NO: 42 - protein, 13 aa, ARTIFICIAL SEQUENCE
  CDRH Sequence
RGGLAARPGGLDV

SEQ ID NO: 43 - protein, 13 aa, ARTIFICIAL SEQUENCE
  CDRL Sequence
SGSSSNIGINYVS

SEQ ID NO: 44 - protein, 11 aa, ARTIFICIAL SEQUENCE
  CDRL Sequence
SGDKLGDKYAC

SEQ ID NO: 45 - protein, 7 aa, ARTIFICIAL SEQUENCE
  CDRL Sequence
DNNKRPL

SEQ ID NO: 46 - protein, 7 aa, ARTIFICIAL SEQUENCE
  CDRL Sequence
QDSKRPF

SEQ ID NO: 47 - protein, 11 aa, ARTIFICIAL SEQUENCE
  CDRL Sequence
GTWDSSLSAWV

SEQ ID NO: 48 - protein, 11 aa, ARTIFICIAL SEQUENCE
  CDRL Sequence
GTWDSNLSAWV

SEQ ID NO: 49 - protein, 9 aa, ARTIFICIAL SEQUENCE
  CDRL Sequence
QAWDSSTAV

SEQ ID NO: 50 - protein, 11 aa, ARTIFICIAL SEQUENCE
  CDRL Sequence
SGDKLGDKNAC

SEQ ID NO: 51 - protein, 7 aa, ARTIFICIAL SEQUENCE
  CDRL Sequence
QDSKRPL

SEQ ID NO: 52 - protein, 9 aa, ARTIFICIAL SEQUENCE
  CDRL Sequence
QAWDISTVV

SEQ ID NO: 53 - DNA, 15 bp, ARTIFICIAL SEQUENCE
  Coding Sequence for CDRH
agttatggtatcagc

SEQ ID NO: 54 - DNA, 15 bp, ARTIFICIAL SEQUENCE
  Coding Sequence for CDRH
agctatggcatgcac

SEQ ID NO: 55 - DNA, 51 bp, ARTIFICIAL SEQUENCE
  Coding Sequence for CDRH
tggatcagcgcttacaatggtaatacaaactatgcacagaagttccagggc

SEQ ID NO: 56 - DNA, 51 bp, ARTIFICIAL SEQUENCE
  Coding Sequence for CDRH
gtcatatggcatgatggaagtaataaatactatgcagactccgtgaagggc

SEQ ID NO: 57 - DNA, 48 bp, ARTIFICIAL SEQUENCE
  Coding Sequence for CDRH
gaagttcgtctggcagctcgtcctgaccattactacggtatggacgtc

SEQ ID NO: 58 - DNA, 45 bp, ARTIFICIAL SEQUENCE
  Coding Sequence for CDRH
gtcgttcccgataactggaaccaggactactacggtatggacgtc

SEQ ID NO: 59 - DNA, 15 bp, ARTIFICIAL SEQUENCE
  Coding Sequence for CDRH
agctatggcatgcac

SEQ ID NO: 60 - DNA, 51 bp, ARTIFICIAL SEQUENCE
  Coding Sequence for CDRH
gctatatggtatgatggaagtaataaatactatgcagagtccgtgaagggc

SEQ ID NO: 61 - DNA, 39 bp, ARTIFICIAL SEQUENCE
  Coding Sequence for CDRH
agggggggtctggcagctcgtccgggcggtctggacgtc

SEQ ID NO: 62 - DNA, 39 bp, ARTIFICIAL SEQUENCE
  Coding Sequence for CDRL
tctggaagcagctccaacattggcattaattatgtatcc

SEQ ID NO: 63 - DNA, 33 bp, ARTIFICIAL SEQUENCE
  Coding Sequence for CDRL
tctggagataaattgggggataaatatgcttgc

SEQ ID NO: 64 - DNA, 21 bp, ARTIFICIAL SEQUENCE
  Coding Sequence for CDRL
gacaataataagcgaccctta

SEQ ID NO: 65 - DNA, 21 bp, ARTIFICIAL SEQUENCE
  Coding Sequence for CDRL
caagatagcaagcggcccttt

SEQ ID NO: 66 - DNA, 33 bp, ARTIFICIAL SEQUENCE
  Coding Sequence for CDRL
ggaacatgggatagcagcctgagtgcttgggtg

SEQ ID NO: 67 - DNA, 33 bp, ARTIFICIAL SEQUENCE
  Coding Sequence for CDRL
ggaacatgggatagcaacctgagtgcttgggtt

SEQ ID NO: 68 - DNA, 27 bp, ARTIFICIAL SEQUENCE
  Coding Seqeunce for CDRL
caggcgtgggacagtagcactgcggta

SEQ ID NO: 69 - DNA, 33 bp, ARTIFICIAL SEQUENCE
  Coding Sequence for CDRL
tctggagataaattgggggataaaaatgcttgc

SEQ ID NO: 70 - DNA, 21 bp, ARTIFICIAL SEQUENCE
  Coding Sequence for CDRL
caggatagcaagcggccctta

SEQ ID NO: 71 - DNA, 27 bp, ARTIFICIAL SEQUENCE
  Coding Sequence for CDRL
caggcgtgggacatcagcactgtggta

SEQ ID NO: 72 - protein, 6 aa, ARTIFICIAL SEQUENCE
  Antigen Binding Protein Coding Sequence
HHHHHH

SEQ ID NO: 73 - protein, 449 aa, Homo sapiens
EVQLVESGGGLVKPGGSLRLSCAASGFTFSSYSMNWVRQAPGKGLEWVSSISSSSSYISYADSVKGRFTISRDNAKNSLYLQMNSLRAEDTAVYFCARDYDFWSAYYDAFDVWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 74 - protein, 216 aa, Homo sapiens
QSVLTQPPSVSGAPGQRVTISCTGSSSNIGAGYDVHWYQQLPGTAPKLLISGNSNRPSGVPDRFSGSKSGTSASLAITGLQAEDEADYYCQSYDSSLSGSVFGGGTKLTVLQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 75 - protein, 441 aa, Homo sapiens
QVQLVQSGAEVKKPGASVKVSCKASGYTLTSYGISWVRQAPGQGLEWMGWISFYNGNTNYAQKVQGRVTMTTDTSTSTVYMELRSLRSDDTAVYYCARGYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 76 - protein, 214 aa, Homo sapiens
QSALTQPASVSGSPGQSITISCTGTSSDVGGYNSVSWYQQHPGKPPKLMIYEVSNRPSGVSIRFSGSKSGNTASLTISGLQAEDEADYFCSSYTSTSMVFGGGTKLTVLQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

(19) United States
(12) Patent Application Publication
(10) Pub. No.: US 2016/0039945 A1
(43) Pub. Date: Feb. 11, 2016

(54) HUMAN ANTIGEN BINDING PROTEINS THAT BIND TO PROPROTEIN CONVERTASE SUBTILISIN KEXIN TYPE 9

(71) Applicant: AMGEN INC., Thousand Oaks, CA (US)

(72) Inventors: Chadwick Terence King, North Vancouver (CA); Simon Mark Jackson, San Carlos, CA (US)

(73) Assignee: AMGEN INC., Thousand Oaks, CA (US)

(21) Appl. No.: 14/777,371

(22) PCT Filed: Mar. 12, 2014
(86) PCT No.: PCT/US14/24702

§ 371 Date: Sep. 15, 2015

Related U.S. Application Data
(60) Provisional application No. 61/798,970, filed on Mar. 15, 2013.

(51) Int. Cl. C07K 16/40; A61K 45/06; G01N 33/573; A61K 39/395

(52) U.S. Cl. CPC C07K 16/40; A61K 39/3955; A61K 45/06; G01N 33/573; C07K 2317/51; C07K 2317/515; G01N 2333/96433

(57) ABSTRACT

The present invention provides compositions and methods relating to or derived from antigen binding proteins capable of inhibiting PCSK9 binding to LDLR. In embodiments, the antigen binding proteins specifically bind PCSK9. In some embodiments, an antigen binding protein is a fully human, humanized, or chimeric antibodies, binding fragments and derivatives of such antibodies, and polypeptides that specifically bind PCSK9 Other embodiments provide nucleic acids encoding such antigen binding proteins, and fragments and derivatives thereof, and polypeptides, cells comprising such polynucleotides, methods of making such antigen binding proteins, and fragments and derivatives thereof, and polypeptides, and methods of using such antigen binding proteins, fragments and derivatives thereof, and polypeptides, including methods of treating or diagnosing subjects suffering from hypercholesterolemia and related disorders or conditions.

### FIELD OF THE INVENTION

The present disclosure relates to nucleic acid molecules encoding antigen binding proteins (“APBs”) that bind to proprotein convertase subtilisin kexin type 9 (hereinafter “PCSK9”), as well as pharmaceutical compositions comprising antigen binding proteins that bind to PCSK9, including antigen binding proteins that inhibit the binding of PCSK9 to the LDL receptor, and methods for treating metabolic disorders using such nucleic acids, polypeptides, or pharmaceutical compositions. Diagnostic methods using the antigen binding proteins are also provided.

### BACKGROUND

Proprotein convertase subtilisin kexin type 9 (PCSK9) is a serine protease involved in regulating the levels of the low density lipoprotein receptor (LDLR) protein (Horton et al., 2007; Seidah and Prat, 2007). In vitro experiments have shown that adding PCSK9 to HepG2 cells lowers the levels of cell surface LDLR (Benjannet et al., 2004; Lagace et al., 2006; Maxwell et al., 2005; Park et al., 2004). Experiments with mice have shown that increasing PCSK9 protein levels decreases levels of LDLR protein in the liver (Benjannet et al., 2004; Lagace et al., 2006; Maxwell et al., 2005; Park et al., 2004), while PCSK9 knockout mice have increased levels of LDLR in the liver (Rashid et al., 2005). Additionally, various human PCSK9 mutations that result in either increased or decreased levels of plasma LDL have been identified (Kotowski et al., 2006; Zhao et al., 2006). PCSK9 has been shown to reduce LDL-receptor levels in the liver, resulting in high levels of LDL-cholesterol in the plasma and increased susceptibility to coronary heart disease (Peterson et al., J Lipid Res. 49(7):1595-9 (2008)). Therefore, it would be highly advantageous to produce a therapeutic antagonist of PCSK9 that inhibits the activity of PCSK9 and the corresponding role PCSK9 plays in various disease conditions.

### SUMMARY

The invention is in part based on a variety of antibodies to PCSK9. PCSK9 presents as an important and advantageous therapeutic target, and the invention provides antibodies as therapeutic and diagnostic agents for use in targeting pathological conditions associated with expression and/or activity of PCSK9. Accordingly, the invention provides methods, compositions, kits and articles of manufacture related to PCSK9.

In one embodiment, an isolated anti-PCSK9 antigen binding protein, comprising an immunoglobulin heavy chain polypeptide, or functional fragment thereof, and an immunoglobulin light chain polypeptide, or functional fragment thereof, wherein the anti-PCSK9 antigen binding protein competes for binding to human PCSK9 with an 101F7, 103B7 or 131D12 antigen binding protein and inhibits the binding of 101F7, 103B7 or 131D12 antigen binding protein to cleaved, mature human PCSK9 (amino acids 31 to 692 of the amino acid sequence of SEQ ID NO:2) by at least. 75%, 80%, 85%, 90%, 95%, 97% or 99% are provided. In a further embodiment an isolated anti-PCSK9 antigen binding protein 1 that competes for binding to human PCSK9 with both antibody 31H4 and antibody 26H5 and inhibits the binding of both 31H4 and 26H5 to cleaved, mature human PCSK9 (amino acids 31 to 692 of the amino acid sequence of SEQ ID NO:2) by at least. 75%, 80%, 85%, 90%, 95%, 97% or 99% are provided.

In a further embodiment an isolated anti-PCSK9 antigen binding protein of any of the preceding ABPs comprising an immunoglobulin heavy chain variable domain polypeptide, or functional fragment thereof having at least 85% sequence identity with any one of SEQ ID NO: 20, 21, 22, or 23 is provided. In a further embodiment an isolated anti-PCSK9 antigen binding protein of any of the above described ABPs comprising an immunoglobulin heavy chain variable domain polypeptide, or functional fragment thereof having at least 90% sequence identity with any one of SEQ ID NO: 20, 21, 22, or 23 are provided. In a further embodiment, an isolated anti-PCSK9 antigen binding protein of any of the above described ABPs comprising an immunoglobulin heavy chain variable domain polypeptide, or functional fragment thereof having at least 95% sequence identity with any one of SEQ ID NO: 20, 21, 22, or 23 is provided. In a further embodiment, an isolated anti-PCSK9 antigen binding protein of any of the above described ABPs comprising an immunoglobulin heavy chain variable domain polypeptide, or functional fragment thereof having the amino acid sequence according to any one of SEQ ID NO: 20, 21, 22, or 23 is provided. In a further embodiment an isolated anti-PCSK9 antigen binding protein of any of the above-described ABPs comprising an immunoglobulin light chain variable domain polypeptide, or functional fragment thereof having at least 85% sequence identity with any one of SEQ ID NO: 16, 17, 18 or 19 is provided. In a further embodiment, an isolated anti-PCSK9 antigen binding protein of any of the above described ABPs comprising an immunoglobulin light chain variable domain polypeptide, or functional fragment thereof having at least 90% sequence identity with any one of SEQ ID NO: 16, 17, 18 or 19 is provided. In a further embodiment, an isolated anti-PCSK9 antigen binding protein of any of the above described ABPs comprising an immunoglobulin light chain variable domain polypeptide, or functional fragment thereof having at least 95% sequence identity with any one of SEQ ID NO: 16, 17, 18 or 19 is provided. In a further embodiment, an isolated anti-PCSK9 antigen binding protein of any of the above described ABPs comprising an immunoglobulin light chain variable domain polypeptide, or functional fragment thereof having the amino acid sequence according to any one of SEQ ID NO: 16, 17, 18 or 19 is provided.

In a further embodiment, an isolated anti-PCSK9 antigen binding protein of any of the above described ABPs comprising at least one, two, three, four, five or six CDRs having the amino acid sequence of SEQ ID NOs: 34 to 52 are provided. In a further embodiment, an isolated anti-PCSK9 antigen binding protein of any of the above described ABPs comprising CDRS comprising amino acid sequences according to SEQ ID Nos 43, 45, 47, 53, 55 and 57 are provided. In a further embodiment, an isolated anti-PCSK9 antigen binding protein of any of the above described ABPs comprising CDRS comprising amino acid sequences according to SEQ ID Nos 43, 45, 48, 53, 55 and 57 are provided. In a further embodiment, an isolated anti-PCSK9 antigen binding protein of any of the above described ABPs comprising CDRS comprising amino acid sequences according to SEQ ID Nos 44, 46, 49, 54, 56 and 58 are provided. In a further embodiment, an isolated anti-PCSK9 antigen binding protein of any of the above described ABPs comprising CDRS comprising amino acid sequences according to SEQ ID Nos 50, 51, 52, 59, 60, and 61 are provided

In a further embodiment, an anti-PCSK9 antigen binding protein of any of the above described ABPS, wherein the antigen binding protein is a monoclonal antibody is provided. In a further embodiment, an anti-PCSK9 antigen binding protein of any of the above described ABPS, wherein the antibody is humanized is provided. In a further embodiment, an anti-PCSK9 antibody of any of the above described antibodies, wherein the antibody is human is provided. In a further embodiment, an anti-PCSK9 antibody of any of the above described antibodies, wherein the antibody is an antibody fragment selected from a Fab, Fab′-SH, Fv, scFv or (Fab′)₂ fragment is provided. In a further embodiment, an anti-PCSK9 antibody of any of the above described antibodies, wherein at least a portion of the framework sequence is a human consensus framework sequence is provided.

In a further embodiment, an isolated nucleic acid encoding an anti-PCSK9 antigen binding protein of any of the above described ABPs is provided. In a further embodiment, a vector comprising the nucleic acid encoding an above described ABP is provided. In one embodiment, the vector of the invention is an expression vector. In another embodiment, a host cell comprising the vector of the invention is provided. In one embodiment host cell of the invention is a prokaryotic host cell. In another embodiment of the invention, the host cell is a eukaryotic host cell. In a further embodiment, a method for making an anti-PCSK9 antigen binding protein of the invention, said method comprising culturing a host cell comprising a vector comprising a nucleic acid encoding an above described anti-PCSK9 antigen binding protein 1 under conditions suitable for expression of the nucleic acid encoding the anti-PCSK9 antibody is provided. In a further embodiment the method of the invention, further comprising recovering the anti-PCSK9 antigen binding protein from the host cell is provided.

In another embodiment, a pharmaceutical composition comprising an above described anti-PCSK9 antigen binding protein and a pharmaceutically acceptable carrier is provided. In a further embodiment, a method of reducing LDL-cholesterol level in a subject, said method comprising administering to the subject an effective amount of any of the above described anti-PCSK9 antigen binding proteins is provided. In a further embodiment, a method of treating cholesterol related disorder in a subject, said method comprising administering to the subject an effective amount of any of the above-described anti-PCSK9 antigen binding proteins is provided. In a further embodiment, a method of treating hypercholesterolemia in a subject, said method comprising administering to the subject an effective amount of the any of the above-described anti-PCSK9 antigen binding proteins is provided. In another embodiment, the above described method of treatment further comprising administering to the subject an effective amount of a second medicament, wherein the anti-PCSK9 antigen binding protein is the first medicament is provided. In some embodiments a method wherein the second medicament elevates the level of LDLR is provided. In some embodiments a method wherein the second medicament reduces the level of LDL-cholesterol is provided. In some embodiments, a method wherein the second medicament comprises a statin is provided. In some embodiments, a method wherein the statin is selected from the group consisting of atorvastatin, fluvastatin, lovastatin, mevastatin, pitavastatin, pravastatin, rosuvastatin, simvastatin, and any combination thereof, is provided. In another embodiment, a method of inhibiting binding of PCSK9 to LDLR in a subject, said method comprising administering to the subject an effective amount of any of the above described anti-PCSK9 antigen binding proteins is provided.

In a further embodiment, a method of detecting PCSK9 protein in a sample, said method comprising (a) contacting the sample with any of the above described antigen binding proteins and (b) detecting formation of a complex between the anti-PCSK9 antigen binding protein and the PCSK9 protein is provided.

### DETAILED DESCRIPTION

The section headings used herein are for organizational purposes only and are not to be construed as limiting the subject matter described.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

Generally, nomenclatures used in connection with, and techniques of, cell and tissue culture, molecular biology, immunology, microbiology, genetics and protein and nucleic acid chemistry and hybridization described herein are those well known and commonly used in the art. The methods and techniques of the present application are generally performed according to conventional methods well known in the art and as described in various general and more specific references that are cited and discussed throughout the present specification unless otherwise indicated. See, e.g., Sambrook et al., Molecular Cloning: A Laboratory Manual, 3^{rd} ed., Cold Spring Harbor Laboratory Press, Cold Spring Harbor, N.Y. (2001) and subsequent editions, Ausubel et al., Current Protocols in Molecular Biology, Greene Publishing Associates (1992), and Harlow & Lane, Antibodies: A Laboratory Manual, Cold Spring Harbor Laboratory Press, Cold Spring Harbor, N.Y. (1988), which are incorporated herein by reference. Enzymatic reactions and purification techniques are performed according to manufacturer's specifications, as commonly accomplished in the art or as described herein. The terminology used in connection with, and the laboratory procedures and techniques of, analytical chemistry, synthetic organic chemistry, and medicinal and pharmaceutical chemistry described herein are those well known and commonly used in the art. Standard techniques can be used for chemical syntheses, chemical analyses, pharmaceutical preparation, formulation, and delivery, and treatment of patients.

It should be understood that the instant disclosure is not limited to the particular methodology, protocols, and reagents, etc., described herein and as such can vary. The terminology used herein is for the purpose of describing particular embodiments only, and is not intended to limit the scope of the present disclosure.

Other than in the operating examples, or where otherwise indicated, all numbers expressing quantities of ingredients or reaction conditions used herein should be understood as modified in all instances by the term “about.” The term “about” when used in connection with percentages can mean±5%, e.g., 1%, 2%, 3%, or 4%.

### I. DEFINITIONS

As used herein, the terms “a” and “an” mean “one or more” unless specifically stated otherwise.

As used herein, an “antigen binding protein” is a protein comprising a portion that binds to an antigen or target and, optionally, a scaffold or framework portion that allows the antigen binding portion to adopt a conformation that promotes binding of the antigen binding protein to the antigen. Examples of antigen binding proteins include a human antibody, a humanized antibody; a chimeric antibody; a recombinant antibody; a single chain antibody; a diabody; a triabody; a tetrabody; a Fab fragment; a F(ab′)₂ fragment; an IgD antibody; an IgE antibody; an IgM antibody; an IgG1 antibody; an IgG2 antibody; an IgG3 antibody; or an IgG4 antibody, and fragments thereof. The antigen binding protein can comprise, for example, an alternative protein scaffold or artificial scaffold with grafted CDRs or CDR derivatives. Such scaffolds include, but are not limited to, antibody-derived scaffolds comprising mutations introduced to, for example, stabilize the three-dimensional structure of the antigen binding protein as well as wholly synthetic scaffolds comprising, for example, a biocompatible polymer. See, e.g., Korndorfer et al., (2003) Proteins: Structure, Function, and Bioinformatics, 53(1):121-129; Roque et al., (2004) Biotechnol. Prog. 20:639-654. In addition, peptide antibody mimetics (“PAMs”) can be used, as well as scaffolds based on antibody mimetics utilizing fibronectin components as a scaffold.

An antigen binding protein can have, for example, the structure of a naturally occurring immunoglobulin. An “immunoglobulin” is a tetrameric molecule. In a naturally occurring immunoglobulin, each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology 2^{nd} ed. Ch. 7 (Paul, W., ed., Raven Press, N.Y. (1989)), incorporated by reference in its entirety for all purposes. The variable regions of each light/heavy chain pair form the antibody binding site such that an intact immunoglobulin has two binding sites.

Naturally occurring immunoglobulin chains exhibit the same general structure of relatively conserved framework regions (FR) joined by three hypervariable regions, also called complementarity determining regions or CDRs. From N-terminus to C-terminus, both light and heavy chains comprise the domains FR1, CDR1, FR2, CDR2, FR3, CDR3 and FR4. The assignment of amino acids to each domain can be done in accordance with the definitions of Kabat et al., (1991) “Sequences of Proteins of Immunological Interest”, 5^{th} Ed., US Dept. of Health and Human Services, PHS, NIH, NIH Publication no. 91-3242. Although presented herein using the Kabat nomenclature system, as desired, the CDRs disclosed herein can also be redefined according an alternative nomenclature scheme, such as that of Chothia (see Chothia & Lesk, (1987) J. Mol. Biol. 196:901-917; Chothia et al., (1989) Nature 342:878-883 or Honegger & Pluckthun, (2001) J. Mol. Biol. 309:657-670).

In the context of the instant disclosure an antigen binding protein is said to “specifically bind” or “selectively bind” its target antigen when the dissociation constant (K_{D}) is ≦10⁻⁸ M. The antibody specifically binds antigen with “high affinity” when the K_{D} is ≦5×10⁻⁹ M, and with “very high affinity” when the K_{D} is ≦5×10⁻¹⁰ M. In one embodiment, the antibodies will bind to PCSK9 with a K_{D} of between about 10⁻⁷ M and 10⁻¹² M, and in yet another embodiment the antibodies will bind with a K_{D}≦5×10⁻⁹.

An “antibody” refers to an intact immunoglobulin or to an antigen binding portion thereof that competes with the intact antibody for specific binding, unless otherwise specified. Antigen binding portions can be produced by recombinant DNA techniques or by enzymatic or chemical cleavage of intact antibodies. Antigen binding portions include, inter alia, Fab, Fab′, F(ab′)₂, Fv, domain antibodies (dAbs), fragments including complementarity determining regions (CDRs), single-chain antibodies (scFv), chimeric antibodies, diabodies, triabodies, tetrabodies, and polypeptides that contain at least a portion of an immunoglobulin that is sufficient to confer specific antigen binding to the polypeptide.

A Fab fragment is a monovalent fragment having the V_{L}, V_{H}, C_{L} and C_{H}1 domains; a F(ab′)₂ fragment is a bivalent fragment having two Fab fragments linked by a disulfide bridge at the hinge region; a Fd fragment has the V_{H} and C_{H}1 domains; an Fv fragment has the V_{L} and V_{H} domains of a single arm of an antibody; and a dAb fragment has a V_{H} domain, a V_{L} domain, or an antigen-binding fragment of a V_{H} or V_{L} domain (U.S. Pat. Nos. 6,846,634, and 6,696,245; and US App. Pub. Nos. 05/0202512, 04/0202995, 04/0038291, 04/0009507, 03/0039958, Ward et al., Nature 341:544-546 (1989)).

A single-chain antibody (scFv) is an antibody in which a V_{L} and a V_{H} region are joined via a linker (e.g., a synthetic sequence of amino acid residues) to form a continuous protein chain wherein the linker is long enough to allow the protein chain to fold back on itself and form a monovalent antigen binding site (see, e.g., Bird et al., (1988) Science 242:423-26 and Huston et al., (1988) Proc. Natl. Acad. Sci. USA 85:5879-83). Diabodies are bivalent antibodies comprising two polypeptide chains, wherein each polypeptide chain comprises V_{H} and V_{L} domains joined by a linker that is too short to allow for pairing between two domains on the same chain, thus allowing each domain to pair with a complementary domain on another polypeptide chain (see, e.g., Holliger et al., (1993) Proc. Natl. Acad. Sci. USA 90:6444-48, and Poljak et al., (1994) Structure 2:1121-23). If the two polypeptide chains of a diabody are identical, then a diabody resulting from their pairing will have two identical antigen binding sites. Polypeptide chains having different sequences can be used to make a diabody with two different antigen binding sites. Similarly, tribodies and tetrabodies are antibodies comprising three and four polypeptide chains, respectively, and forming three and four antigen binding sites, respectively, which can be the same or different.

Complementarity determining regions (CDRs) and framework regions (FR) of a given antibody can be identified using the system described by Kabat et al., (1991) “Sequences of Proteins of Immunological Interest”, 5^{th} Ed., US Dept. of Health and Human Services, PHS, NIH, NIH Publication no. 91-3242. Although presented using the Kabat nomenclature system, as desired, the CDRs disclosed herein can also be redefined according an alternative nomenclature scheme, such as that of Chothia (see Chothia & Lesk, (1987) J. Mol. Biol. 196:901-917; Chothia et al., (1989) Nature 342:878-883 or Honegger & Pluckthun, (2001) J. Mol. Biol. 309:657-670). One or more CDRs can be incorporated into a molecule either covalently or noncovalently to make it an antigen binding protein. An antigen binding protein can incorporate the CDR(s) as part of a larger polypeptide chain, can covalently link the CDR(s) to another polypeptide chain, or can incorporate the CDR(s) noncovalently. The CDRs permit the antigen binding protein to specifically bind to a particular antigen of interest.

An antigen binding protein can but need not have one or more binding sites. If there is more than one binding site, the binding sites can be identical to one another or can be different. For example, a naturally occurring human immunoglobulin typically has two identical binding sites, while a “bispecific” or “bifunctional” antibody has two different binding sites. Antigen binding proteins of this bispecific form (e.g., those comprising various heavy and light chain CDRs provided herein) comprise aspects of the instant disclosure.

The term “human antibody” includes all antibodies that have one or more variable and constant regions derived from human immunoglobulin sequences. In one embodiment, all of the variable and constant domains are derived from human immunoglobulin sequences (a fully human antibody). These antibodies can be prepared in a variety of ways, examples of which are described below, including through the immunization with an antigen of interest of a mouse that is genetically modified to express antibodies derived from human heavy and/or light chain-encoding genes, such as a mouse derived from a XENOMOUSE®, ULTIMAB™, HUMAB-MOUSE®, VELOCIMOUSE®, VELOCIMMUNE®, KYMOUSE, or ALIVAMAB system, or derived from human heavy chain transgenic mouse, transgenic rat human antibody repertoire, transgenic rabbit human antibody repertoire or cow human antibody repertoire or HUTARG™ technology. Phage-based approaches can also be employed.

A humanized antibody has a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject. In one embodiment, certain amino acids in the framework and constant domains of the heavy and/or light chains of the non-human species antibody are mutated to produce the humanized antibody. In another embodiment, the constant domain(s) from a human antibody are fused to the variable domain(s) of a non-human species. In another embodiment, one or more amino acid residues in one or more CDR sequences of a non-human antibody are changed to reduce the likely immunogenicity of the non-human antibody when it is administered to a human subject, wherein the changed amino acid residues either are not critical for immunospecific binding of the antibody to its antigen, or the changes to the amino acid sequence that are made are conservative changes, such that the binding of the humanized antibody to the antigen is not significantly worse than the binding of the non-human antibody to the antigen. Examples of how to make humanized antibodies can be found in U.S. Pat. Nos. 6,054,297, 5,886,152 and 5,877,293.

The term “chimeric antibody” refers to an antibody that contains one or more regions from one antibody and one or more regions from one or more other antibodies. In one embodiment, one or more of the CDRs are derived from a human antibody that binds to PCSK9. In another embodiment, all of the CDRs are derived from a human antibody that binds to PCSK9. In another embodiment, the CDRs from more than one human antibody that binds to PCSK9 are mixed and matched in a chimeric antibody. For instance, a chimeric antibody can comprise a CDR1 from the light chain of a first human antibody that binds to PCSK9, a CDR2 and a CDR3 from the light chain of a second human antibody that binds to PCSK9, and the CDRs from the heavy chain from a third antibody that binds to PCSK9. Further, the framework regions can be derived from one of the same antibodies that binds PCSK9, from one or more different antibodies, such as a human antibody, or from a humanized antibody. In one example of a chimeric antibody, a portion of the heavy and/or light chain is identical with, homologous to, or derived from an antibody from a particular species or belonging to a particular antibody class or subclass, while the remainder of the chain(s) is/are identical with, homologous to, or derived from an antibody or antibodies from another species or belonging to another antibody class or subclass. Also included are fragments of such antibodies that exhibit the desired biological activity (e.g., the ability to specifically bind to PCSK9).

The term “light chain” includes a full-length light chain and fragments thereof having sufficient variable region sequence to confer binding specificity. A full-length light chain includes a variable region domain, V_{L}, and a constant region domain, C_{L}. The variable region domain of the light chain is at the amino-terminus of the polypeptide. Light chains include kappa (“κ”) chains and lambda (“λ”) chains.

The term “heavy chain” includes a full-length heavy chain and fragments thereof having sufficient variable region sequence to confer binding specificity. A full-length heavy chain includes a variable region domain, V_{H}, and three constant region domains, C_{H}1, C_{H}2, and C_{H}3. The V_{H} domain is at the amino-terminus of the polypeptide, and the C_{H} domains are at the carboxyl-terminus, with the C_{H}3 being closest to the carboxy-terminus of the polypeptide. Heavy chains can be of any isotype, including IgG (including IgG1, IgG2, IgG3 and IgG4 subtypes), IgA (including IgA1 and IgA2 subtypes), IgM and IgE.

The term “immunologically functional fragment” (or simply “fragment”) of an antigen binding protein, e.g., an antibody or immunoglobulin chain (heavy or light chain), as used herein, is an antigen binding protein comprising a portion (regardless of how that portion is obtained or synthesized) of an antibody that lacks at least some of the amino acids present in a full-length chain but which is capable of specifically binding to an antigen. Such fragments are biologically active in that they bind specifically to the target antigen and can compete with other antigen binding proteins, including intact antibodies, for specific binding to a given epitope. In one aspect, such a fragment will retain at least one CDR present in the full-length light or heavy chain, and in some embodiments will comprise a single heavy chain and/or light chain or portion thereof. These biologically active fragments can be produced by recombinant DNA techniques, or can be produced by enzymatic or chemical cleavage of antigen binding proteins, including intact antibodies. Immunologically functional immunoglobulin fragments include, but are not limited to, Fab, Fab′, F(ab′)₂, Fv, domain antibodies and single-chain antibodies, and can be derived from any mammalian source, including but not limited to human, mouse, rat, camelid or rabbit. It is contemplated further that a functional portion of the antigen binding proteins disclosed herein, for example, one or more CDRs, could be covalently bound to a second protein or to a small molecule to create a therapeutic agent directed to a particular target in the body, possessing bifunctional therapeutic properties, or having a prolonged serum half-life.

An “Fc” region contains two heavy chain fragments comprising the C_{H}2 and C_{H}3 domains of an antibody. The two heavy chain fragments are held together by two or more disulfide bonds and by hydrophobic interactions of the C_{H}3 domains.

An “Fab′ fragment” contains one light chain and a portion of one heavy chain that contains the V_{H} domain and the C_{H}1 domain and also the region between the C_{H}1 and C_{H}2 domains, such that an interchain disulfide bond can be formed between the two heavy chains of two Fab′ fragments to form an F(ab′)₂ molecule.

An “F(ab′)₂ fragment” contains two light chains and two heavy chains containing a portion of the constant region between the C_{H}1 and C_{H}2 domains, such that an interchain disulfide bond is formed between the two heavy chains. A F(ab′)₂ fragment thus is composed of two Fab′ fragments that are held together by a disulfide bond between the two heavy chains.

The “Fv region” comprises the variable regions from both the heavy and light chains, but lacks the constant regions.

A “domain antibody” is an immunologically functional immunoglobulin fragment containing only the variable region of a heavy chain or the variable region of a light chain. In some instances, two or more V_{H} regions are covalently joined with a peptide linker to create a bivalent domain antibody. The two V_{H} regions of a bivalent domain antibody can target the same or different antigens.

A “hemibody” is an immunologically-functional immunoglobulin construct comprising a complete heavy chain, a complete light chain and a second heavy chain Fc region paired with the Fc region of the complete heavy chain. A linker can, but need not, be employed to join the heavy chain Fc region and the second heavy chain Fc region. In particular embodiments a hemibody is a monovalent form of an antigen binding protein disclosed herein. In other embodiments, pairs of charged residues can be employed to associate one Fc region with the second Fc region.

A “bivalent antigen binding protein” or “bivalent antibody” comprises two antigen binding sites. In some instances, the two binding sites have the same antigen specificities. Bivalent antigen binding proteins and bivalent antibodies can be bispecific, as described herein, and form aspects of the instant disclosure.

A “multispecific antigen binding protein” or “multispecific antibody” is one that targets more than one antigen or epitope, and forms another aspect of the instant disclosure.

A “bispecific,” “dual-specific” or “bifunctional” antigen binding protein or antibody is a hybrid antigen binding protein or antibody, respectively, having two different antigen binding sites. Bispecific antigen binding proteins and antibodies are a species of multispecific antigen binding protein or multispecific antibody and can be produced by a variety of methods including, but not limited to, fusion of hybridomas or linking of Fab′ fragments. See, e.g., Songsivilai and Lachmann, (1990) Clin. Exp. Immunol. 79:315-321; Kostelny et al., (1992) J. Immunol. 148:1547-1553. The two binding sites of a bispecific antigen binding protein or antibody will bind to two different epitopes, which can reside on the same (e.g., PCSK9) or different protein targets, including (e.g., : lecithin cholesterol acyl transferase (LCAT), angiopoietin protein like-3 (ANGPTL3), ANGPTL4, Endothelial Lipase (EL), apolipoprotein CIII (ApoCIII), lipoprotein lipase (LPL), fibroblast growth factor 21 (FGF21)).

The term “polynucleotide” or “nucleic acid” includes both single-stranded and double-stranded nucleotide polymers. The nucleotides comprising the polynucleotide can be ribonucleotides or deoxyribonucleotides or a modified form of either type of nucleotide. Said modifications include base modifications such as bromouridine and inosine derivatives, ribose modifications such as 2′,3′-dideoxyribose, and internucleotide linkage modifications such as phosphorothioate, phosphorodithioate, phosphoroselenoate, phosphorodiselenoate, phosphoroanilothioate, phoshoraniladate and phosphoroamidate.

The term “oligonucleotide” means a polynucleotide comprising 200 or fewer nucleotides. In some embodiments, oligonucleotides are 10 to 60 bases in length. In other embodiments, oligonucleotides are 12, 13, 14, 15, 16, 17, 18, 19, or 20 to 40 nucleotides in length. Oligonucleotides can be single stranded or double stranded, e.g., for use in the construction of a mutant gene. Oligonucleotides can be sense or antisense oligonucleotides. An oligonucleotide can include a label, including a radiolabel, a fluorescent label, a hapten or an antigenic label, for detection assays. Oligonucleotides can be used, for example, as PCR primers, cloning primers or hybridization probes.

An “isolated nucleic acid molecule” means a DNA or RNA of genomic, mRNA, cDNA, or synthetic origin or some combination thereof which is not associated with all or a portion of a polynucleotide in which the isolated polynucleotide is found in nature, or is linked to a polynucleotide to which it is not linked in nature. For purposes of this disclosure, it is understood that “a nucleic acid molecule comprising” a particular nucleotide sequence does not encompass intact chromosomes. Isolated nucleic acid molecules “comprising” specified nucleic acid sequences can include, in addition to the specified sequences, coding sequences for up to ten or even up to twenty other proteins or portions thereof, or can include operably linked regulatory sequences that control expression of the coding region of the recited nucleic acid sequences, and/or can include vector sequences.

Unless specified otherwise, the left-hand end of any single-stranded polynucleotide sequence discussed herein is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream sequences;” sequence regions on the DNA strand having the same sequence as the RNA transcript that are 3′ to the 3′ end of the RNA transcript are referred to as “downstream sequences.”

The term “control sequence” refers to a polynucleotide sequence that can affect the expression and processing of coding sequences to which it is ligated. The nature of such control sequences can depend upon the host organism. In particular embodiments, control sequences for prokaryotes can include a promoter, a ribosomal binding site, and a transcription termination sequence. For example, control sequences for eukaryotes can include promoters comprising one or a plurality of recognition sites for transcription factors, transcription enhancer sequences, and transcription termination sequence. “Control sequences” can include leader sequences and/or fusion partner sequences.

The term “vector” means any molecule or entity (e.g., nucleic acid, plasmid, bacteriophage or virus) used to transfer protein coding information into a host cell.

The term “expression vector” or “expression construct” refers to a vector that is suitable for transformation of a host cell and contains nucleic acid sequences that direct and/or control (in conjunction with the host cell) expression of one or more heterologous coding regions operatively linked thereto. An expression construct can include, but is not limited to, sequences that affect or control transcription, translation, and, if introns are present, affect RNA splicing of a coding region operably linked thereto.

As used herein, “operably linked” means that the components to which the term is applied are in a relationship that allows them to carry out their inherent functions under suitable conditions. For example, a control sequence in a vector that is “operably linked” to a protein coding sequence is ligated thereto so that expression of the protein coding sequence is achieved under conditions compatible with the transcriptional activity of the control sequences.

The term “host cell” means a cell that has been transformed, or is capable of being transformed, with a nucleic acid sequence and thereby expresses a gene of interest. The term includes the progeny of the parent cell, whether or not the progeny is identical in morphology or in genetic make-up to the original parent cell, so long as the gene of interest is present.

The term “transduction” means the transfer of genes from one bacterium to another, usually by bacteriophage. “Transduction” also refers to the acquisition and transfer of eukaryotic cellular sequences by replication-defective retroviruses.

The term “transfection” means the uptake of foreign or exogenous DNA by a cell, and a cell has been “transfected” when the exogenous DNA has been introduced inside the cell membrane. A number of transfection techniques are well known in the art and are disclosed herein. See, e.g., Graham et al., (1973) Virology 52:456; Sambrook et al., (2001), supra; Davis et al., (1986) Basic Methods in Molecular Biology, Elsevier; Chu et al., (1981) Gene 13:197. Such techniques can be used to introduce one or more exogenous DNA moieties into suitable host cells.

The term “transformation” refers to a change in a cell's genetic characteristics, and a cell has been transformed when it has been modified to contain new DNA or RNA. For example, a cell is transformed where it is genetically modified from its native state by introducing new genetic material via transfection, transduction, or other techniques. Following transfection or transduction, the transforming DNA can recombine with that of the cell by physically integrating into a chromosome of the cell, or can be maintained transiently as an episomal element without being replicated, or can replicate independently as a plasmid. A cell is considered to have been “stably transformed” when the transforming DNA is replicated with the division of the cell.

The terms “polypeptide” or “protein” are used interchangeably herein to refer to a polymer of amino acid residues. The terms also apply to amino acid polymers in which one or more amino acid residues is an analog or mimetic of a corresponding naturally occurring amino acid, as well as to naturally occurring amino acid polymers. The terms can also encompass amino acid polymers that have been modified, e.g., by the addition of carbohydrate residues to form glycoproteins, or phosphorylated. Polypeptides and proteins can be produced by a naturally-occurring and non-recombinant cell, or polypeptides and proteins can be produced by a genetically-engineered or recombinant cell. Polypeptides and proteins can comprise molecules having the amino acid sequence of a native protein, or molecules having deletions from, additions to, and/or substitutions of one or more amino acids of the native sequence. The terms “polypeptide” and “protein” encompass antigen binding proteins that specifically or selectively bind to PCSK9, or sequences that have deletions from, additions to, and/or substitutions of one or more amino acids of an antigen binding protein that specifically or selectively binds to PCSK9. The term “polypeptide fragment” refers to a polypeptide that has an amino-terminal deletion, a carboxyl-terminal deletion, and/or an internal deletion as compared with the full-length protein. Such fragments can also contain modified amino acids as compared with the full-length protein. In certain embodiments, fragments are about five to 500 amino acids long. For example, fragments can be at least 5, 6, 8, 10, 14, 20, 50, 70, 100, 110, 150, 200, 250, 300, 350, 400, or 450 amino acids long. Useful polypeptide fragments include immunologically functional fragments of antibodies, including binding domains. In the case of an antigen binding protein that binds to PCSK9, useful fragments include but are not limited to a CDR region, a variable domain of a heavy or light chain, a portion of an antibody chain or just its variable region including two CDRs, and the like.

The term “isolated protein” referred means that a subject protein (1) is free of at least some other proteins with which it would normally be found, (2) is essentially free of other proteins from the same source, e.g., from the same species, (3) is expressed by a cell from a different species, (4) has been separated from at least about 50 percent of polynucleotides, lipids, carbohydrates, or other materials with which it is associated in nature, (5) is operably associated (by covalent or noncovalent interaction) with a polypeptide with which it is not associated in nature, or (6) does not occur in nature. Typically, an “isolated protein” constitutes at least about 5%, at least about 10%, at least about 25%, or at least about 50% of a given sample. Genomic DNA, cDNA, mRNA or other RNA, of synthetic origin, or any combination thereof can encode such an isolated protein. Preferably, the isolated protein is substantially free from proteins or polypeptides or other contaminants that are found in its natural environment that would interfere with its therapeutic, diagnostic, prophylactic, research or other use.

A “variant” of a polypeptide (e.g., an antigen binding protein, or an antibody) comprises an amino acid sequence wherein one or more amino acid residues are inserted into, deleted from and/or substituted into the amino acid sequence relative to another polypeptide sequence. Variants include fusion proteins.

A “derivative” of a polypeptide is a polypeptide (e.g., an antigen binding protein, or an antibody) that has been chemically modified in some manner distinct from insertion, deletion, or substitution variants, e.g., by conjugation to another chemical moiety.

The term “naturally occurring” as used throughout the specification in connection with biological materials such as polypeptides, nucleic acids, host cells, and the like, refers to materials which are found in nature.

“Antigen binding region” means a protein, or a portion of a protein, that specifically binds a specified antigen, e.g. PCSK9. For example, that portion of an antigen binding protein that contains the amino acid residues that interact with an antigen and confer on the antigen binding protein its specificity and affinity for the antigen is referred to as “antigen binding region.” An antigen binding region typically includes one or more “complementary binding regions” (“CDRs”). Certain antigen binding regions also include one or more “framework” regions. A “CDR” is an amino acid sequence that contributes to antigen binding specificity and affinity. “Framework” regions can aid in maintaining the proper conformation of the CDRs to promote binding between the antigen binding region and an antigen.

In certain aspects, recombinant antigen binding proteins that bind to PCSK9, are provided. In this context, a “recombinant protein” is a protein made using recombinant techniques, i.e., through the expression of a recombinant nucleic acid as described herein. Methods and techniques for the production of recombinant proteins are well known in the art.

The term “compete” when used in the context of antigen binding proteins (e.g., neutralizing antigen binding proteins, neutralizing antibodies, agonistic antigen binding proteins, agonistic antibodies and binding proteins that bind to PCSK9 that compete for the same epitope or binding site on a target means competition between antigen binding proteins as determined by an assay in which the antigen binding protein (e.g., antibody or immunologically functional fragment thereof) under study prevents or inhibits the specific binding of a reference molecule (e.g., a reference ligand, or reference antigen binding protein, such as a reference antibody) to a common antigen (e.g., PCSK9 or a fragment thereof). Numerous types of competitive binding assays can be used to determine if a test molecule competes with a reference molecule for binding. Examples of assays that can be employed include solid phase direct or indirect radioimmunoassay (RIA), solid phase direct or indirect enzyme immunoassay (EIA), sandwich competition assay (see, e.g., Stahli et al., (1983) Methods in Enzymology 9:242-253); solid phase direct biotin-avidin EIA (see, e.g., Kirkland et al., (1986) J. Immunol. 137:3614-3619) solid phase direct labeled assay, solid phase direct labeled sandwich assay (see, e.g., Harlow and Lane, (1988) supra); solid phase direct label RIA using I-125 label (see, e.g., Morel et al., (1988) Molec. Immunol. 25:7-15); solid phase direct biotin-avidin EIA (see, e.g., Cheung, et al., (1990) Virology 176:546-552); and direct labeled RIA (Moldenhauer et al., (1990) Scand. J. Immunol. 32:77-82). Typically, such an assay involves the use of a purified antigen bound to a solid surface or cells bearing either of an unlabelled test antigen binding protein or a labeled reference antigen binding protein. Competitive inhibition is measured by determining the amount of label bound to the solid surface or cells in the presence of the test antigen binding protein. Usually the test antigen binding protein is present in excess. Antigen binding proteins identified by competition assay (competing antigen binding proteins) include antigen binding proteins binding to the same epitope as the reference antigen binding proteins and antigen binding proteins binding to an adjacent epitope sufficiently proximal to the epitope bound by the reference antigen binding protein for steric hindrance to occur. Additional details regarding methods for determining competitive binding are provided in the examples herein. Usually, when a competing antigen binding protein is present in excess, it will inhibit specific binding of a reference antigen binding protein to a common antigen by at least 40%, 45%, 50%, 55%, 60%, 65%, 70% or 75%. In some instance, binding is inhibited by at least 80%, 85%, 90%, 95%, or 97% or more.

The term “antigen” refers to a molecule or a portion of a molecule capable of being bound by a selective binding agent, such as an antigen binding protein (including, e.g., an antibody or immunological functional fragment thereof), and may also be capable of being used in an animal to produce antibodies capable of binding to that antigen. An antigen can possess one or more epitopes that are capable of interacting with different antigen binding proteins, e.g., antibodies.

The term “epitope” means the amino acids of a target molecule that are contacted by an antigen binding protein (for example, an antibody) when the antigen binding protein is bound to the target molecule. The term includes any subset of the complete list of amino acids of the target molecule that are contacted when an antigen binding protein, such as an antibody, is bound to the target molecule. An epitope can be contiguous or non-contiguous (e.g., (i) in a single-chain polypeptide, amino acid residues that are not contiguous to one another in the polypeptide sequence but that within in context of the target molecule are bound by the antigen binding protein, or (ii) in a multimeric receptor comprising two or more individual components, amino acid residues that are present on one or more of the individual components, but which are still bound by the antigen binding protein). In certain embodiments, epitopes can be mimetic in that they comprise a three dimensional structure that is similar to an antigenic epitope used to generate the antigen binding protein, yet comprise none or only some of the amino acid residues found in that epitope used to generate the antigen binding protein. Most often, epitopes reside on proteins, but in some instances can reside on other kinds of molecules, such as nucleic acids. Epitope determinants can include chemically active surface groupings of molecules such as amino acids, sugar side chains, phosphoryl or sulfonyl groups, and can have specific three dimensional structural characteristics, and/or specific charge characteristics. Generally, antigen binding proteins specific for a particular target molecule will preferentially recognize an epitope on the target molecule in a complex mixture of proteins and/or macromolecules.

The term “identity” refers to a relationship between the sequences of two or more polypeptide molecules or two or more nucleic acid molecules, as determined by aligning and comparing the sequences. “Percent identity” means the percent of identical residues between the amino acids or nucleotides in the compared molecules and is calculated based on the size of the smallest of the molecules being compared. For these calculations, gaps in alignments (if any) must be addressed by a particular mathematical model or computer program (i.e., an “algorithm”). Methods that can be used to calculate the identity of the aligned nucleic acids or polypeptides include those described in Computational Molecular Biology, (Lesk, A. M., ed.), (1988) New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., (1987) Sequence Analysis in Molecular Biology, New York: Academic Press; Sequence Analysis Primer, (Gribskov, M. and Devereux, J., eds.), 1991, New York: M. Stockton Press; and Carillo et al., (1988) J. Applied Math. 48:1073.

In calculating percent identity, the sequences being compared are aligned in a way that gives the largest match between the sequences. The computer program used to determine percent identity is the GCG program package, which includes GAP (Devereux et al., (1984) Nucl. Acid Res. 12:387; Genetics Computer Group, University of Wisconsin, Madison, Wis.). The computer algorithm GAP is used to align the two polypeptides or polynucleotides for which the percent sequence identity is to be determined. The sequences are aligned for optimal matching of their respective amino acid or nucleotide (the “matched span”, as determined by the algorithm). A gap opening penalty (which is calculated as 3× the average diagonal, wherein the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. In certain embodiments, a standard comparison matrix (see, Dayhoff et al., (1978) Atlas of Protein Sequence and Structure 5:345-352 for the PAM 250 comparison matrix; Henikoff et al., (1992) Proc. Natl. Acad. Sci. U.S.A. 89:10915-10919 for the BLOSUM 62 comparison matrix) is also used by the algorithm.

Recommended parameters for determining percent identity for polypeptides or nucleotide sequences using the GAP program are the following:

Algorithm: Needleman et al., 1970, J. Mol. Biol. 48:443-453;

Comparison matrix: BLOSUM 62 from Henikoff et al., 1992, supra;

Gap Penalty: 12 (but with no penalty for end gaps)

Gap Length Penalty: 4

Threshold of Similarity: 0

Certain alignment schemes for aligning two amino acid sequences can result in matching of only a short region of the two sequences, and this small aligned region can have very high sequence identity even though there is no significant relationship between the two full-length sequences. Accordingly, the selected alignment method (e.g., the GAP program) can be adjusted if so desired to result in an alignment that spans at least 50 contiguous amino acids of the target polypeptide.

As used herein, “substantially pure” means that the described species of molecule is the predominant species present, that is, on a molar basis it is more abundant than any other individual species in the same mixture. In certain embodiments, a substantially pure molecule is a composition wherein the object species comprises at least 50% (on a molar basis) of all macromolecular species present. In other embodiments, a substantially pure composition will comprise at least 80%, 85%, 90%, 95%, or 99% of all macromolecular species present in the composition. In other embodiments, the object species is purified to essential homogeneity wherein contaminating species cannot be detected in the composition by conventional detection methods and thus the composition consists of a single detectable macromolecular species.

The terms “treat” and “treating” refer to any indicia of success in the treatment or amelioration of an injury, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. For example, certain methods presented herein can be employed to treat dyslipidemia, either prophylactically or as an acute treatment, to decrease circulating cholesterol levels and/or ameliorate a symptom associated with primary hyperlipidemia (heterozygous familial and non-familial), mixed dyslipidemia, and homozygous familial hypercholesterolemia.

An “effective amount” is generally an amount sufficient to reduce the severity and/or frequency of symptoms, eliminate the symptoms and/or underlying cause, prevent the occurrence of symptoms and/or their underlying cause, and/or improve or remediate the damage that results from or is associated with diabetes, obesity and dyslipidemia. In some embodiments, the effective amount is a therapeutically effective amount or a prophylactically effective amount. A “therapeutically effective amount” is an amount sufficient to remedy a disease state (e.g., diabetes, obesity or dyslipidemia) or symptoms, particularly a state or symptoms associated with the disease state, or otherwise prevent, hinder, retard or reverse the progression of the disease state or any other undesirable symptom associated with the disease in any way whatsoever. A “prophylactically effective amount” is an amount of a pharmaceutical composition that, when administered to a subject, will have the intended prophylactic effect, e.g., preventing or delaying the onset (or reoccurrence) of diabetes, obesity or dyslipidemia, or reducing the likelihood of the onset (or reoccurrence) of diabetes, obesity or dyslipidemia or associated symptoms. The full therapeutic or prophylactic effect does not necessarily occur by administration of one dose, and may occur only after administration of a series of doses. Thus, a therapeutically or prophylactically effective amount can be administered in one or more administrations.

“Amino acid” takes its normal meaning in the art. The twenty naturally-occurring amino acids and their abbreviations follow conventional usage. See, Immunology—A Synthesis, 2^{nd} Edition, (E. S. Golub and D. R. Green, eds.), Sinauer Associates: Sunderland, Mass. (1991), incorporated herein by reference for any purpose. Stereoisomers (e.g., D-amino acids) of the twenty conventional amino acids, unnatural or non-naturally occurring or encoded amino acids such as α-,α-disubstituted amino acids, N-alkyl amino acids, and other unconventional amino acids can also be suitable components for polypeptides and are included in the phrase “amino acid.” Examples of non-natural and non-naturally encoded amino acids (which can be substituted for any naturally-occurring amino acid found in any sequence disclosed herein, as desired) include: 4-hydroxyproline, γ-carboxyglutamate, ε-N,N,N-trimethyllysine, ε-N-acetyllysine, ε-phosphoserine, N-acetylserine, N-formylmethionine, 3-methylhistidine, 5-hydroxylysine, σ-N-methylarginine, and other similar amino acids and imino acids (e.g., 4-hydroxyproline). In the polypeptide notation used herein, the left-hand direction is the amino terminal direction and the right-hand direction is the carboxyl-terminal direction, in accordance with standard usage and convention. A non-limiting lists of examples of non-naturally occurring/encoded amino acids that can be inserted into an antigen binding protein sequence or substituted for a wild-type residue in an antigen binding sequence include β-amino acids, homoamino acids, cyclic amino acids and amino acids with derivatized side chains. Examples include (in the L-form or D-form; abbreviated as in parentheses): citrulline (Cit), homocitrulline (hCit), Nα-methylcitrulline (NMeCit), Nα-methylhomocitrulline (Nα-MeHoCit), ornithine (Orn), Nα-Methylornithine (Nα-MeOrn or NMeOrn), sarcosine (Sar), homolysine (hLys or hK), homoarginine (hArg or hR), homoglutamine (hQ), Nα-methylarginine (NMeR), Nα-methylleucine (Nα-MeL or NMeL), N-methylhomolysine (NMeHoK), Nα-methylglutamine (NMeQ), norleucine (Nle), norvaline (Nva), 1,2,3,4-tetrahydroisoquinoline (Tic), Octahydroindole-2-carboxylic acid (Oic), 3-(1-naphthyl)alanine (1-Nal), 3-(2-naphthyl)alanine (2-Nal), 1,2,3,4-tetrahydroisoquinoline (Tic), 2-indanylglycine (IgI), para-iodophenylalanine (pI-Phe), para-aminophenylalanine (4AmP or 4-Amino-Phe), 4-guanidino phenylalanine (Guf), glycyllysine (abbreviated “K(Nε-glycyl)” or “K(glycyl)” or “K(gly)”), nitrophenylalanine (nitrophe), aminophenylalanine (aminophe or Amino-Phe), benzylphenylalanine (benzylphe), γ-carboxyglutamic acid (γ-carboxyglu), hydroxyproline (hydroxypro), p-carboxyl-phenylalanine (Cpa), α-aminoadipic acid (Aad), Nα-methyl valine (NMeVal), N-α-methyl leucine (NMeLeu), Nα-methylnorleucine (NMeNle), cyclopentylglycine (Cpg), cyclohexylglycine (Chg), acetylarginine (acetylarg), α,β-diaminopropionoic acid (Dpr), α,γ-diaminobutyric acid (Dab), diaminopropionic acid (Dap), cyclohexylalanine (Cha), 4-methyl-phenylalanine (MePhe), β,β-diphenyl-alanine (BiPhA), aminobutyric acid (Abu), 4-phenyl-phenylalanine (or biphenylalanine; 4Bip), α-amino-isobutyric acid (Aib), beta-alanine, beta-aminopropionic acid, piperidinic acid, aminocaprioic acid, aminoheptanoic acid, aminopimelic acid, desmosine, diaminopimelic acid, N-ethylglycine, N-ethylaspargine, hydroxylysine, allo-hydroxylysine, isodesmosine, allo-isoleucine, N-methylglycine, N-methylisoleucine, N-methylvaline, 4-hydroxyproline (Hyp), γ-carboxyglutamate, ε-N,N,N-trimethyllysine, ε-N-acetyllysine, O-phosphoserine, N-acetylserine, N-formylmethionine, 3-methylhistidine, 5-hydroxylysine, ω-methylarginine, 4-Amino-O-Phthalic Acid (4APA), and other similar amino acids, and derivatized forms of any of those specifically listed.

### II. GENERAL OVERVIEW

Antigen-binding proteins that bind to PCSK9 with extended in vivo half lives are provided herein. In some embodiments, the antigen binding proteins of the invention having extended half lives are pH sensitive binders. In some embodiments, the antigen binding proteins of the invention having extended half lives comprise mutations in their constant domains. In some embodiments, the antigen binding proteins of the invention having extended half lives are pH sensitive binders and comprise mutations in their constant domains.

In some embodiments of the present disclosure the antigen binding proteins provided can comprise polypeptides into which one or more complementary determining regions (CDRs) can be embedded and/or joined. In such antigen binding proteins, the CDRs can be embedded into a “framework” region, which orients the CDR(s) such that the proper antigen binding properties of the CDR(s) is achieved. In general, such antigen binding proteins that are provided inhibit the binding of PCSK9 to the LDLR, Accordingly, the antigen binding proteins provided herein and offer potential therapeutic benefit for the range of conditions which hypercholesterolemia, primary hyperlipidemia (heterozygous familial and non-familial), mixed dyslipidemia, homozygous familial hypercholesterolemia, cardiovascular disease, and broadly any disease or condition in which it is desirable to inhibit in vivo the binding of PCSK9 to LDLR.

Certain antigen binding proteins described herein are antibodies or are derived from antibodies. In certain embodiments, the polypeptide structure of the antigen binding proteins is based on antibodies, including, but not limited to, monoclonal antibodies, bispecific antibodies, minibodies, domain antibodies, synthetic antibodies (sometimes referred to herein as “antibody mimetics”), chimeric antibodies, humanized antibodies, human antibodies, antibody fusions (sometimes referred to herein as “antibody conjugates”), hemibodies and fragments thereof. The various structures are further described herein below.

The antigen binding proteins provided herein have been demonstrated to bind PCSK9 (e.g. human PCSK9). The antigen binding proteins that specifically bind to PCSK9 that are disclosed herein have a variety of utilities. Some of the antigen binding proteins, for instance, are useful in specific binding assays, in the affinity purification of PCSK9, including the human PCSK9, and in screening assays to identify other inhibitors of PCSK9 binding to LDLR.

The antigen binding proteins that specifically bind to PCSK9 that are disclosed herein can be used in a variety of treatment applications, as explained herein. For example, certain antigen binding proteins are useful for treating conditions associated with elevated cholesterol levels in a patient, such as reducing, alleviating, or treating dyslipidemia and cardiovascular disease. Other uses for the antigen binding proteins include, for example, diagnosis of diseases or conditions associated with PCSK9 and screening assays to determine the presence or absence of PCSK9. Some of the antigen binding proteins described herein can be useful in treating conditions, symptoms and/or the pathology associated with increased cholesterol levels. Exemplary conditions include, but are not limited to, dyslipidemia and cardiovascular disease.

### PCSK9

The antigen binding proteins disclosed herein inhibit the binding of PCSK9 to LDLR as defined herein. In vivo, the mature form of PCSK9 is the active form of the molecule. The nucleotide sequence encoding full length human PCSK9 is provided; the nucleotides encoding the pro-domain sequence are underlined.

| Accession number NM_174936 | |
|---|---|
| (SEQ ID NO: 1) | |
| ATGGGCACCG TCAGCTCCAG GCGGTCCTGG TGGCCGCTGC CACTGCTGCT GCTGCTGCTG | 60 |
| CTGCTCCTGG GTCCCGCGGG CGCCCGTGCG CAGGAGGACG AGGACGGCGA CTACGAGGAG | 120 |
| CTGGTGCTAG CCTTGCGCTC CGAGGAGGAC GGCCTGGCCG AAGCACCCGA GCACGGAACC | 180 |
| ACAGCCACCT TCCACCGCTG CGCCAAGGAT CCGTGGAGGT TGCCTGGCAC CTACGTGGTG | 240 |
| GTGCTGAAGG AGGAGACCCA CCTCTCGCAG TCAGAGCGCA CTGCCCGCCG CCTGCAGGCC | 300 |
| CAGGCTGCCC GCCGGGGATA CCTCACCAAG ATCCTGCATG TCTTCCATGG CCTTCTTCCT | 360 |
| GGCTTCCTGG TGAAGATGAG TGGCGACCTG CTGGAGCTGG CCTTGAAGTT GCCCCATGTC | 420 |
| GACTACATCG AGGAGGACTC CTCTGTCTTT GCCCAGAGCA TCCCGTGGAA CCTGGAGCGG | 480 |
| ATTACCCCTC CGCGGTACCG GGCGGATGAA TACCAGCCCC CCGACGGAGG CAGCCTGGTG | 540 |
| GAGGTGTATC TCCTAGACAC CAGCATACAG AGTGACCACC GGGAAATCGA GGGCAGGGTC | 600 |
| ATGGTCACCG ACTTCGAGAA TGTGCCCGAG GAGGACGGGA CCCGCTTCCA CAGACAGGCC | 660 |
| AGCAAGTGTG ACAGTCATGG CACCCACCTG GCAGGGGTGG TCAGCGGCCG GGATGCCGGC | 720 |
| GTGGCCAAGG GTGCCAGCAT GCGCAGCCTG CGCGTGCTCA ACTGCCAAGG GAAGGGCACG | 780 |
| GTTAGCGGCA CCCTCATAGG CCTGGAGTTT ATTCGGAAAA GCCAGCTGGT CCAGCCTGTG | 840 |
| GGGCCACTGG TGGTGCTGCT GCCCCTGGCG GGTGGGTACA GCCGCGTCCT CAACGCCGCC | 900 |
| TGCCAGCGCC TGGCGAGGGC TGGGGTCGTG CTGGTCACCG CTGCCGGCAA CTTCCGGGAC | 960 |
| GATGCCTGCC TCTACTCCCC AGCCTCAGCT CCCGAGGTCA TCACAGTTGG GGCCACCAAT | 1020 |
| GCCCAGGACC AGCCGGTGAC CCTGGGGACT TTGGGGACCA ACTTTGGCCG CTGTGTGGAC | 1080 |
| CTCTTTGCCC CAGGGGAGGA CATCATTGGT GCCTCCAGCG ACTGCAGCAC CTGCTTTGTG | 1140 |
| TCACAGAGTG GGACATCACA GGCTGCTGCC CACGTGGCTG GCATTGCAGC CATGATGCTG | 1200 |
| TCTGCCGAGC CGGAGCTCAC CCTGGCCGAG TTGAGGCAGA GACTGATCCA CTTCTCTGCC | 1260 |
| AAAGATGTCA TCAATGAGGC CTGGTTCCCT GAGGACCAGC GGGTACTGAC CCCCAACCTG | 1320 |
| GTGGCCGCCC TGCCCCCCAG CACCCATGGG GCAGGTTGGC AGCTGTTTTG CAGGACTGTG | 1380 |
| TGGTCAGCAC ACTCGGGGCC TACACGGATG GCCACAGCCA TCGCCCGCTG CGCCCCAGAT | 1440 |
| GAGGAGCTGC TGAGCTGCTC CAGTTTCTCC AGGAGTGGGA AGCGGCGGGG CGAGCGCATG | 1500 |
| GAGGCCCAAG GGGGCAAGCT GGTCTGCCGG GCCCACAACG CTTTTGGGGG TGAGGGTGTC | 1560 |
| TACGCCATTG CCAGGTGCTG CCTGCTACCC CAGGCCAACT GCAGCGTCCA CACAGCTCCA | 1620 |
| CCAGCTGAGG CCAGCATGGG GACCCGTGTC CACTGCCACC AACAGGGCCA CGTCCTCACA | 1680 |
| GGCTGCAGCT CCCACTGGGA GGTGGAGGAC CTTGGCACCC ACAAGCCGCC TGTGCTGAGG | 1740 |
| CCACGAGGTC AGCCCAACCA GTGCGTGGGC CACAGGGAGG CCAGCATCCA CGCTTCCTGC | 1800 |
| TGCCATGCCC CAGGTCTGGA ATGCAAAGTC AAGGAGCATG GAATCCCGGC CCCTCAGGGG | 1860 |
| CAGGTGACCG TGGCCTGCGA GGAGGGCTGG ACCCTGACTG GCTGCAGCGC CCTCCCTGGG | 1920 |
| ACCTCCCACG TCCTGGGGGC CTACGCCGTA GACAACACGT GTGTAGTCAG GAGCCGGGAC | 1980 |
| GTCAGCACTA CAGGCAGCAC CAGCGAAGAG GCCGTGACAG CCGTTGCCAT CTGCTGCCGG | 2040 |
| AGCCGGCACC TGGCGCAGGC CTCCCAGGAG CTCCAG | |

The amino acid sequence of full length human PCSK9 is provided; the amino acids that make up the pro-domain sequence are underlined:

Accession number NP_777596

(SEQ ID NO: 2)

MGTVSSRRSWWPLPLLLLLLLLLGPAGARA
QEDEDGDYEELVLALRSEEDGLAEAPEHGTTATFHRCAKDPWRLPGTYVV
VLKEETHLSQSERTARRLQAQAARRGYLTKILHVFHGLLPGFLVKMSGDL
LELALKLPHVDYIEEDSSVFAQSIPWNLERITPPRYRADEYQPPDGGSLV
EVYLLDTSIQSDHREIEGRVMVTDFENVPEEDGTRFHRQASKCDSHGTH
LAGVVSGRDAGVAKGASMRSLRVLNCQGKGTVSGTLIGLEFIRKSQLVQ
PVGPLVVLLPLAGGYSRVLNAACQRLARAGVVLVTAAGNFRDDACLYS
PASAPEVITVGATNAQDQPVTLGTLGTNFGRCVDLFAPGEDIIGASSDC
STCFVSQSGTSQAAAHVAGIAAMMLSAEPELTLAELRQRLIHFSAKDVI
NEAWFPEDQRVLTPNLVAALPPSTHGAGWQLFCRTVWSAHSGPTRM
ATAIARCAPDEELLSCSSFSRSGKRRGERMEAQGGKLVCRAHNAFG
GEGVYAIARCCLLPQANCSVHTAPPAEASMGTRVHCHQQGHVLTGC
SSHWEVEDLGTHKPPVLRPRGQPNQCVGHREASIHASCCHAPGLE
CKVKEHGIPAPQGQVTVACEEGWTLTGCSALPGTSHVLGAYAVDN
TCVVRSRDVSTTGSTSEEAVTAVAICCRSRHLAQASQELQ

The nucleotide sequence encoding full length cynomolgus PCSK9 is provided; the nucleotides encoding the pro-domain sequence are underlined.

(SEQ ID NO: 3)

ATGGGTACCGTCAGCTCCAGGCGGTCCTGGTGGCCTCTGCCGCTGCCACT
GCTGCTGCTCCTGCTCCTGGGTCCCGCTGGCGCCCGTGCGCAGGAGGACG
AGGACGGCGACTACGAGGAGCTGGTGCTAGCCTTGCGTTCCGAGGAGGAC
GGCCTGGCCGACGCACCCGAGCACGGAGCCACAGCCACCTTCCACCGCTG
CGCCAAGGATCCGTGGAGGTTGCCCGGCACCTACGTGGTGGTGCTGAAGG
AGGAGACCCACCGCTCGCAGTCAGAGCGCACTGCCCGCCGCCTGCAGGCC
CAAGCTGCCCGCCGGGGATACCTCACCAAGATCCTGCATGTCTTCCATCA
CCTTCTTCCTGGCTTCCTGGTGAAGATGAGTGGCGACCTGCTGGAGCTGG
CCCTGAAGTTGCCCCATGTCGACTACATCGAGGAGGACTCCTCTGTCTTC
GCCCAGAGCATCCCATGGAACCTGGAGCGAATTACTCCTGCACGGTACCG
GGCGGATGAATACCAGCCCCCCAAAGGAGGCAGCCTGGTGGAGGTGTATC
TCCTAGACACCAGCATACAGAGTGACCACCGGGAAATCGAGGGCAGGGTC
ATGGTCACCGACTTCGAGAGTGTGCCCGAGGAGGACGGGACCCGCTTCCA
CAGACAGGCCAGCAAGTGTGACAGCCATGGCACCCACCTGGCAGGGGTGG
TCAGCGGCCGGGATGCCGGCGTGGCCAAGGGCGCCGGCCTGCGTAGCCTG
CGCGTGCTCAACTGCCAAGGGAAGGGCACGGTCAGCGGCACCCTCATAGG
CCTGGAGTTTATTCGGAAAAGCCAGCTGGTCCAGCCCGTGGGGCCACTGG
TTGTGCTGCTGCCCCTGGCGGGTGGGTACAGCCGGGTCTTCAACGCCGCC
TGCCAGCGCCTGGCGAGGGCTGGGGTCGTGCTGGTCACCGCTGCCGGCAA
CTTCCGGGACGATGCCTGCCTCTACTCTCCAGCCTCGGCTCCCGAGGTCA
TCACAGTTGGGGCCACCAATGCCCAGGACCAGCCGGTGACCCTGGGGACT
TTGGGGACCAACTTTGGCCGCTGTGTGGACCTCTTTGCCCCAGGGGAGGA
CATCATTGGTGCCTCCAGCGACTGCAGCACCTGCTTTGTGTCACGGAGTG
GGACATCGCAGGCTGCTGCCCACGTGGCTGGCATTGCAGCCATGATGCTG
TCTGCCGAGCCGGAGCTCACTCTGGCCGAGTTGAGGCAGAGACTGATCCA
CTTCTCTGCCAAAGATGTCATCAATGAGGCCTGGTTCCCTGAGGACCAGC
GGGTACTGACCCCCAACCTGGTGGCCGCCCTGCCCCCCAGCACCCACAGG
GCAGGTTGGCAGCTGTTTTGCAGGACTGTGTGGTCAGCACACTCGGGTCC
TACACGGATGGCCACAGCCGTAGCCCGCTGCGCCCAGGATGAGGAGCTGC
TGAGCTGCTCCAGTTTCTCCAGGAGTGGGAAGCGGCGGGGCGAGCGCATC
GAGGCCCAAGGGGGCAAGCGGGTCTGCCGGGCCCACAACGCTTTTGGGGG
TGAGGGTGTCTACGCCATTGCCAGGTGCTGCCTGCTACCCCAGGTCAACT
GCAGCGTCCACACAGCTCCACCAGCTGGGGCCAGCATGGGGACCCGTGTC
CACTGCCATCAGCAGGGCCACGTCCTCACAGGCTGCAGCTCCCACTGGGA
GGTGGAGGACCTTGGCACCCACAAGCCGCCTGTGCTGAGGCCACGAGGTC
AGCCCAACCAGTGTGTGGGCCACAGGGAGGCCAGCATCCACGCTTCCTGC
TGCCATGCCCCAGGTCTGGAATGCAAAGTCAGGGAGCATGGAATCCCGGC
CCCTCAGGAGCAGGTTATCGTGGCCTGTGAGGACGGCTGGACCCTGACCG
GCTGCAGTGCCCTCCCTGGGACCTCCCATGTCCTGGGGGCCTACGCTGTA
GACAACACGTGTGTGGTCAGGAGCCGGGACGTCAGCACCACAGGCAGCAC
CAGCGAAGAAGCCGTGGCAGCCGTTGCCATCTGCTGCCGGAGCCGGCACC
TGGTGCAGGCCTCCCAGGAGCTCCAG

The amino acid sequence of full length cynomolgous PCSK9 is provided; the amino acids that make up the pro-domain sequence are underlined:

(SEQ ID NO: 4)
MGTVSSRRSWWPLPLPLLLLLLLGPAGARA
QEDEDGDYEELVLALRSEEDGLADAPEHGATATFHRCAKDPWRLPGTYVV
VLKEETHRSQSERTARRLQAQAARRGYLTKILHVFHHLLPGFLVKMSGDL
LELALKLPHVDYIEEDSSVFAQSIPWNLERITPARYRADEYQPPKGGSLV
EVYLLDTSIQSDHREIEGRVMVTDFESVPEEDGTRFHRQASKCDSHGT
HLAGVVSGRDAGVAKGAGLRSLRVLNCQGKGTVSGTLIGLEFIRKSQL
VQPVGPLVVLLPLAGGYSRVFNAACQRLARAGVVLVTAAGNFRDDAC
LYSPASAPEVITVGATNAQDQPVTLGTLGTNFGRCVDLFAPGEDIIGASS
DCSTCFVSRSGTSQAAAHVAGIAAMMLSAEPELTLAELRQRLIHFSAKD
VINEAWFPEDQRVLTPNLVAALPPSTHRAGWQLFCRTVWSAHSGPTR
MATAVARCAQDEELLSCSSFSRSGKRRGERIEAQGGKRVCRAHNAF
GGEGVYAIARCCLLPQVNCSVHTAPPAGASMGTRVHCHQQGHVLTG
CSSHWEVEDLGTHKPPVLRPRGQPNQCVGHREASIHASCCHAPGL
ECKVREHGIPAPQEQVIVACEDGWTLTGCSALPGTSHVLGAYAVDN
TCVVRSRDVSTTGSTSEEAVAAVAICCRSRHLVQASQELQ

As described herein, PCSK9 proteins can also include fragments. The term PCSK9 also includes post-translational modifications of the PCSK9 amino acid sequence, for example, possible N-linked glycosylation sites. Thus, the antigen binding proteins can bind to or be generated from proteins glycosylated at one or more position.

Antigen Binding Proteins that Specifically Bind to PCSK9

A variety of selective binding agents useful for inhibiting PCSK9 binding to LDLR are provided. These agents include, for instance, antigen binding proteins that contain an antigen binding domain (e.g., single chain antibodies, domain antibodies, hemibodies, immunoadhesions, and polypeptides with an antigen binding region) and specifically bind to PCSK9, in particular a human PCSK9.

In general, the antigen binding proteins that are provided typically comprise one or more CDRs as described herein (e.g., 1, 2, 3, 4, 5 or 6 CDRs). In some embodiments the antigen binding proteins are naturally expressed by clones, while in other embodiments, the antigen binding protein can comprise (a) a polypeptide framework structure and (b) one or more CDRs that are inserted into and/or joined to the polypeptide framework structure. In some of these embodiments a CDR forms a component of a heavy or light chains expressed by the clones described herein; in other embodiments a CDR can be inserted into a framework in which the CDR is not naturally expressed. A polypeptide framework structure can take a variety of different forms. For example, a polypeptide framework structure can be, or comprise, the framework of a naturally occurring antibody, or fragment or variant thereof, or it can be completely synthetic in nature. Examples of various antigen binding protein structures are further described below.

In some embodiments in which the antigen binding protein comprises (a) a polypeptide framework structure and (b) one or more CDRs that are inserted into and/or joined to the polypeptide framework structure, the polypeptide framework structure of an antigen binding protein is an antibody or is derived from an antibody, including, but not limited to, monoclonal antibodies, bispecific antibodies, minibodies, domain antibodies, synthetic antibodies (sometimes referred to herein as “antibody mimetics”), chimeric antibodies, humanized antibodies, antibody fusions (sometimes referred to as “antibody conjugates”), and portions or fragments of each, respectively. In some instances, the antigen binding protein is an immunological fragment of an antibody (e.g., a Fab, a Fab′, a F(ab′)₂, or a scFv).

Certain of the antigen binding proteins as provided herein specifically bind to PCSK9, including the human form of this protein. In one embodiment, an antigen binding protein specifically binds human self-cleaved, mature, secreted PCSK9 comprising amino acids 31 to 692 of the amino acid sequence of SEQ ID NO: 2 and inhibits PCSK9 from binding to LDLR.

### Antigen Binding Protein Structure

Some of the antigen binding proteins that specifically bind PCSK9, including the human form provided herein have a structure typically associated with naturally occurring antibodies. The structural units of these antibodies typically comprise one or more tetramers, each composed of two identical couplets of polypeptide chains, though some species of mammals also produce antibodies having only a single heavy chain. In a typical antibody, each pair or couplet includes one full-length “light” chain (in certain embodiments, about 25 kDa) and one full-length “heavy” chain (in certain embodiments, about 50-70 kDa). Each individual immunoglobulin chain is composed of several “immunoglobulin domains,” each consisting of roughly 90 to 110 amino acids and expressing a characteristic folding pattern. These domains are the basic units of which antibody polypeptides are composed. The amino-terminal portion of each chain typically includes a variable domain that is responsible for antigen recognition. The carboxy-terminal portion is more conserved evolutionarily than the other end of the chain and is referred to as the “constant region” or “C region”. Human light chains generally are classified as kappa (“κ”) and lambda (“λ”) light chains, and each of these contains one variable domain and one constant domain. Heavy chains are typically classified as mu, delta, gamma, alpha, or epsilon chains, and these define the antibody's isotype as IgM, IgD, IgG, IgA, and IgE, respectively. IgG has several subtypes, including, but not limited to, IgG1, IgG2, IgG3, and IgG4. IgM subtypes include IgM, and IgM2. IgA subtypes include IgA1 and IgA2. In humans, the IgA and IgD isotypes contain four heavy chains and four light chains; the IgG and IgE isotypes contain two heavy chains and two light chains; and the IgM isotype contains five heavy chains and five light chains. The heavy chain C region typically comprises one or more domains that can be responsible for effector function. The number of heavy chain constant region domains will depend on the isotype. IgG heavy chains, for example, each contain three C region domains known as C_{H}1, C_{H}2 and C_{H}3. The antibodies that are provided can have any of these isotypes and subtypes. In certain embodiments, an antigen binding protein that specifically binds to PCSK9.

In full-length light and heavy chains, the variable and constant regions are joined by a “J” region of about twelve or more amino acids, with the heavy chain also including a “D” region of about ten more amino acids. See, e.g., Fundamental Immunology, 2nd ed., Ch. 7 (Paul, W., ed.) 1989, New York: Raven Press (hereby incorporated by reference in its entirety for all purposes). The variable regions of each light/heavy chain pair typically form the antigen binding site.

One example of an IgG2 heavy constant domain of an exemplary monoclonal antibody that specifically binds to PCSK9 has the amino acid sequence:

(SEQ ID NO: 5)
ASTKGPSVFPLAPCSRSTSESTAALGCLVKDYFPEPVTVSWNSGALTSG
VHTFPAVLQSSGLYSLSSVVTVPSSNFGTQTYTCNVDHKPSNTKVDKTV
ERKCCVECPPCPAPPVAGPSVFLFPPKPKDTLMISRTPEVTCVVVDVS
HEDPEVQFNWYVDGVEVHNAKTKPREEQFNSTFRVVSVLTVVHQDWL
NGKEYKCKVSNKGLPAPIEKTISKTKGQPREPQVYTLPPSREEMTKNQ
VSLTCLVKGFYPSDIAVEWESNGQPENNYKTTPPMLDSDGSFFLYSK
LTVDKSRWQQGNVFSCSVMHEALHNHYTQKSLSLSPGK.

One example of a kappa light constant domain of an exemplary monoclonal antibody that binds to a PCSK9 has the amino acid sequence:

(SEQ ID NO: 6)
RTVAAPSVFIFPPSDEQLKSGTASVVCLLNNFYPREAKVQWKVDNALQ
SGNSQESVTEQDSKDSTYSLSSTLTLSKADYEKHKVYACEVTHQGLS
SPVTKSFNRGEC.

One example of a lambda light constant domain of an exemplary monoclonal antibody that binds to PCSK9 has the amino acid sequence:

(SEQ ID NO: 7)
QPKANPTVTLFPPSSEELQANKATLVCLISDFYPGAVTV
AWKADGSPVKAGVETTKPSKQSNNKYAASSYLSLTPEQWKSHRSYSCQVT
HEGSTVEKTVAPTECS.

Variable regions of immunoglobulin chains generally exhibit the same overall structure, comprising relatively conserved framework regions (FR) joined by three hypervariable regions, more often called “complementarity determining regions” or CDRs. The CDRs from the two chains of each heavy chain/light chain pair mentioned above typically are aligned by the framework regions to form a structure that binds specifically with a specific epitope on the target protein (e.g., PCSK9). From N-terminal to C-terminal, naturally-occurring light and heavy chain variable regions both typically conform with the following order of these elements: FR1, CDR1, FR2, CDR2, FR3, CDR3 and FR4. A numbering system has been devised for assigning numbers to amino acids that occupy positions in each of these domains. This numbering system is defined in Kabat et al., (1991) “Sequences of Proteins of Immunological Interest”, 5^{th} Ed., US Dept. of Health and Human Services, PHS, NIH, NIH Publication no. 91-3242. Although presented using the Kabat nomenclature system, as desired, the CDRs disclosed herein can also be redefined according an alternative nomenclature scheme, such as that of Chothia (see Chothia & Lesk, (1987) J. Mol. Biol. 196:901-917; Chothia et al., (1989) Nature 342:878-883 or Honegger & Pluckthun, (2001) J. Mol. Biol. 309:657-670).

The various heavy chain and light chain variable regions of antigen binding proteins provided herein are depicted in Table 2. Each of these variable regions can be attached to the disclosed heavy and light chain constant regions to form a complete antibody heavy and light chain, respectively. Further, each of the so-generated heavy and light chain sequences can be combined to form a complete antibody structure. It should be understood that the heavy chain and light chain variable regions provided herein can also be attached to other constant domains having different sequences than the exemplary sequences listed above.

Specific examples of some of the full length light and heavy chains of the antibodies that are provided and their corresponding amino acid sequences are summarized in Tables 1A and 1B. Table 1A shows exemplary light chain sequences, and Table 1B shows exemplary heavy chain sequences.

TABLE 1A
Exemplary Antibody Light Chain Sequences

| Contained | | SEQ ID | |
|---|---|---|---|
| in Clone | Designation | NO: | Amino Acid Sequence |
| 101F7 | L1 | 8 | QSVLTQPPSVSAAPGQKVTISCSGSSSNIGIN |
| | | | YVSWYQLLPGTAPKLLIYDNNKRPLGIPDRF |
| | | | SGSKSGTSATLGITGLQTGDETDYYCGTWD |
| | | | SSLSAWVFGGGTKLTVLG |
| | | | QPKANPTVTLFPPSSEELQANKATLVCLISD |
| | | | FYPGAVTV |
| | | | AWKADGSPVKAGVETTKPSKQSNNKYAAS |
| | | | SYLSLTPEQWKSHRSYSCQVT |
| | | | HEGSTVEKTVAPTECS |
| 103B7 | L2 | 9 | QSVLTQPPSVSAAPGQKVTISCSGSSSNIGIN |
| | | | YVSWYQLLPGTAPKLLIYDNNKRPLGIPDRF |
| | | | SGSKSGTSVTLGITGLQTGDEADYFCGTWD |
| | | | SNLSAWVFGGGTKLTVLG |
| | | | QPKANPTVTLFPPSSEELQANKATLVCLISD |
| | | | FYPGAVTV |
| | | | AWKADGSPVKAGVETTKPSKQSNNKYAAS |
| | | | SYLSLTPEQWKSHRSYSCQVT |
| | | | HEGSTVEKTVAPTECS |
| 131D12 | L3 | 10 | SYELTQPPSVSVSPGQTASITCSGDKLGDKY |
| | | | ACWFQQKPGQSPVLVIYQDSKRPFGIPDRFS |
| | | | GSKSGNTATLTISGTQAMEEADYYCQAWD |
| | | | SSTAVFGGGTKLTVLG |
| | | | QPKANPTVTLFPPSSEELQANKATLVCLISD |
| | | | FYPGAVTV |
| | | | AWKADGSPVKAGVETTKPSKQSNNKYAAS |
| | | | SYLSLTPEQWKSHRSYSCQVT |
| | | | HEGSTVEKTVAPTECS |
| 27B2 | L4 | 11 | SYELTQPPSVSVSPGQTISISCSGDKLGDKNA |
| | | | CWYQQKPGQSPGLVIYQDSKRPLGIPARFSG |
| | | | SNSGNTATLTISGTQAMDEADYYCQAWDIS |
| | | | TVVFGGGTKLTVLG |
| | | | QPKANPTVTLFPPSSEELQANKATLVCLISD |
| | | | FYPGAVTV |
| | | | AWKADGSPVKAGVETTKPSKQSNNKYAAS |
| | | | SYLSLTPEQWKSHRSYSCQVT |
| | | | HEGSTVEKTVAPTECS |

TABLE 1B
Exemplary Antibody Heavy Chain Sequences

| Contained | | SEQ ID | |
|---|---|---|---|
| in Clone | Designation | NO: | Amino Acid Sequence |
| 101F7 | H1 | 12 | QVQLVQSGAEVKKPGASVKVSCKASGYTFSSY |
| | | | GISWVRQAPGQGLEWMGWISAYNGNTNYAQ |
| | | | KFQGRVTMTTDTSTSTAYMELRSLRSDDTAVY |
| | | | YCAREVRLAARPDHYYGMDVWGQGTTVTVSS |
| | | | ASTKGPSVFPLAPCSRSTSESTAALGCLVKDYF |
| | | | PEPVTVSWNSGALTSGVHTF |
| | | | PAVLQSSGLYSLSSVVTVPSSNFGTQTYTCNVD |
| | | | HKPSNTKVDKTVERKCC |
| | | | VECPPCPAPPVAGPSVFLFPPKPKDTLMISRTPE |
| | | | VTCVVVDVSHEDPEVQ |
| | | | FNWYVDGVEVHNAKTKPREEQFNSTFRVVSV |
| | | | LTVVHQDWLNGKEYKCKVS |
| | | | NKGLPAPIEKTISKTKGQPREPQVYTLPPSREEM |
| | | | TKNQVSLTCLVKGFYP |
| | | | SDIAVEWESNGQPENNYKTTPPMLDSDGSFFL |
| | | | YSKLTVDKSRWQQGNVFS |
| | | | CSVMHEALHNHYTQKSLSLSPGK |
| 103B7 | H2 | 13 | QVQLVQSGAEVKKPGASVKVSCKASGYTFSSY |
| | | | GISWVRQAPGQGLEWMGWISAYNGNTNYAQ |
| | | | KFQGRVTMTTDTSTSTAYMELRSLRSDDTAVY |
| | | | YCAREVRLAARPDHYYGMDVWGQGTTVTVSS |
| | | | ASTKGPSVFPLAPCSRSTSESTAALGCLVKDYF |
| | | | PEPVTVSWNSGALTSGVHTF |
| | | | PAVLQSSGLYSLSSVVTVPSSNFGTQTYTCNVD |
| | | | HKPSNTKVDKTVERKCC |
| | | | VECPPCPAPPVAGPSVFLFPPKPKDTLMISRTPE |
| | | | VTCVVVDVSHEDPEVQ |
| | | | FNWYVDGVEVHNAKTKPREEQFNSTFRVVSV |
| | | | LTVVHQDWLNGKEYKCKVS |
| | | | NKGLPAPIEKTISKTKGQPREPQVYTLPPSREEM |
| | | | TKNQVSLTCLVKGFYP |
| | | | SDIAVEWESNGQPENNYKTTPPMLDSDGSFFL |
| | | | YSKLTVDKSRWQQGNVFS |
| | | | CSVMHEALHNHYTQKSLSLSPGK |
| 131D12 | H3 | 14 | QGQLVESGGGVVQPGRSLRLSCAASGFTFSSY |
| | | | GMHWVRQAPGKGLEWVAVIWHDGSNKYYAD |
| | | | SVKGRFTISRDNSKNTLYLQMNSLRAEDTAVY |
| | | | YCARVVPDNWNQDYYGMDVWGQGTTVTVSS |
| | | | ASTKGPSVFPLAPCSRSTSESTAALGCLVKDYF |
| | | | PEPVTVSWNSGALTSGVHTF |
| | | | PAVLQSSGLYSLSSVVTVPSSNFGTQTYTCNVD |
| | | | HKPSNTKVDKTVERKCC |
| | | | VECPPCPAPPVAGPSVFLFPPKPKDTLMISRTPE |
| | | | VTCVVVDVSHEDPEVQ |
| | | | FNWYVDGVEVHNAKTKPREEQFNSTFRVVSV |
| | | | LTVVHQDWLNGKEYKCKVS |
| | | | NKGLPAPIEKTISKTKGQPREPQVYTLPPSREEM |
| | | | TKNQVSLTCLVKGFYP |
| | | | SDIAVEWESNGQPENNYKTTPPMLDSDGSFFL |
| | | | YSKLTVDKSRWQQGNVFS |
| | | | CSVMHEALHNHYTQKSLSLSPGK |
| 27B2 | H4 | 15 | QVQLVESGGGVVQPGRSLRLSCAASGFTFSSY |
| | | | GMHWVRQAPGKGLEWVAAIWYDGSNKYYAE |
| | | | SVKGRFTISRDNSKYTLYLQVNSLRAEDTAVY |
| | | | YCARRGGLAARPGGLDVWGQGTTVTV |
| | | | ASTKGPSVFPLAPCSRSTSESTAALGCLVKDYF |
| | | | PEPVTVSWNSGALTSGVHTF |
| | | | PAVLQSSGLYSLSSVVTVPSSNFGTQTYTCNVD |
| | | | HKPSNTKVDKTVERKCC |
| | | | VECPPCPAPPVAGPSVFLFPPKPKDTLMISRTPE |
| | | | VTCVVVDVSHEDPEVQ |
| | | | FNWYVDGVEVHNAKTKPREEQFNSTFRVVSV |
| | | | LTVVHQDWLNGKEYKCKVS |
| | | | NKGLPAPIEKTISKTKGQPREPQVYTLPPSREEM |
| | | | TKNQVSLTCLVKGFYP |
| | | | SDIAVEWESNGQPENNYKTTPPMLDSDGSFFL |
| | | | YSKLTVDKSRWQQGNVFS |
| | | | CSVMHEALHNHYTQKSLSLSPGK |

Each of the exemplary heavy chains (H1, H2, H3 etc.) listed in Table 1B, infra, can be combined with any of the exemplary light chains shown in Table 1A, infra, to form an antibody. Examples of such combinations include H1 combined with any of L1 through L4 H2 combined with any of L1 through L4; H3 combined with any of L1 through L4, H4 combined with any of L1 through L4 and so on. In some instances, the antibodies include at least one heavy chain and one light chain from those listed in Tables 1A and 1B, infra; particular examples pairings of light chains and heavy chains include L1 with H1, L2 with H2, L3 with H3, L4 with H4, L1 with H2, H3 or H4, L2 with H1, H3 or H4, L3 with H2, H1 or H4, L4 with H1, H2 or H3. In addition to antigen binding proteins comprising a heavy and a light chain from the same clone, a heavy chain from a first clone can be paired with a light chain from a second clone (e.g., a heavy chain from a first clone paired with a light chain from a second clone or a heavy chain from a first clone paired with a light chain from a second clone). Generally, such pairings can include VL with 90% or greater homology can be paired with the heavy chain of the naturally occurring clone.

In some instances, the antibodies comprise two different heavy chains and two different light chains listed in Tables 1A and 1B, infra. In other instances, the antibodies contain two identical light chains and two identical heavy chains. As an example, an antibody or immunologically functional fragment can include two L1 light chains with two H1 heavy chains, two L2 light chains with two H1 heavy chains, two L3 light chains with two H2 heavy chains or two H3 heavy chains, as well as other similar combinations of pairs comprising the light chains and pairs of heavy chains as listed in Tables 1A and 1B, infra.

In another aspect of the instant disclosure, “hemibodies” are provided. A hemibody is a monovalent antigen binding protein comprising (i) an intact light chain, and (ii) a heavy chain fused to an Fc region (e.g., an IgG2 Fc region of SEQ ID NO: 5), optionally via a linker, The linker can be a (G₄S)ₓ linker (SEQ ID NO: 77) where “x” is a non-zero integer (e.g., (G₄S)_{2,}(G₄S)_{3,}(G₄S)_{4,}(G₄S)_{5,}(G₄S)_{6,}(G₄S)_{7,}(G₄S)_{8,}(G₄S)_{9,}(G₄S)_{10,}; SEQ ID NOS: 78- 75, respectively). Hemibodies can be constructed using the provided heavy and light chain components.

Other antigen binding proteins that are provided are variants of antibodies formed by combination of the heavy and light chains shown in Tables 1A and 1B, infra and comprise light and/or heavy chains that each have at least 70%, 75%, 80%, 85%, 90%, 95%, 96%, 97%, 98% or 99% identity to the amino acid sequences of these chains. In some instances, such antibodies include at least one heavy chain and one light chain, whereas in other instances the variant forms contain two identical light chains and two identical heavy chains.

### Variable Domains of Antigen Binding Proteins

Also provided are antigen binding proteins that contain an antibody heavy chain variable region selected from the group consisting of V_{H}1, V_{H}2, or V_{H}3 as shown in Table 2B and/or an antibody light chain variable region selected from the group consisting of V_{L}1, V_{L}2, V_{L}3, or V_{L}4 as shown in Table 2A, and immunologically functional fragments, derivatives, muteins and variants of these light chain and heavy chain variable regions.

TABLE 2A
Exemplary Antibody Variable Light (V_{L}) Chains

| Contained | | SEQ ID | |
|---|---|---|---|
| in Clone | Designation | NO. | Amino Acid Sequence |
| 101F7 | V_{L}1 | 16 | QSVLTQPPSVSAAPGQKVTISCSGSSSNIGINYVSWYQL |
| | | | LPGTAPKLLIYDNNKRPLGIPDRFSGSKSGTSATLGITGL |
| | | | QTGDETDYYCGTWDSSLSAWVFGGGTKLTVLG |
| 103B7 | V_{L}2 | 17 | QSVLTQPPSVSAAPGQKVTISCSGSSSNIGINYVSWYQL |
| | | | LPGTAPKLLIYDNNKRPLGIPDRFSGSKSGTSVTLGITGL |
| | | | QTGDEADYFCGTWDSNLSAWVFGGGTKLTVLG |
| 131D12 | V_{L}3 | 18 | SYELTQPPSVSVSPGQTASITCSGDKLGDKYACWFQQK |
| | | | PGQSPVLVIYQDSKRPFGIPDRFSGSKSGNTATLTISGTQ |
| | | | AMEEADYYCQAWDSSTAVFGGGTKLTVLG |
| 27B2 | V_{L}4 | 19 | SYELTQPPSVSVSPGQTISISCSGDKLGDKNACWYQQKP |
| | | | GQSPGLVIYQDSKRPLGIPARFSGSNSGNTATLTISGTQ |
| | | | AMDEADYYCQAWDISTVVFGGGTKLTVLG |

TABLE 2B
Exemplary Antibody Variable Heavy (V_{H}) Chains

| Contained | | SEQ ID | |
|---|---|---|---|
| in Clone | Designation | NO. | Amino Acid Sequence |
| 101F7 | V_{H} 1 | 20 | QVQLVQSGAEVKKPGASVKVSCKASGYTFSSYGIS |
| | | | WVRQAPGQGLEWMGWISAYNGNTNYAQKFQGRV |
| | | | TMTTDTSTSTAYMELRSLRSDDTAVYYCAREVRLA |
| | | | ARPDHYYGMDVWGQGTTVTVSS |
| 103B7 | V_{H} 1 | 21 | QVQLVQSGAEVKKPGASVKVSCKASGYTFSSYGIS |
| | | | WVRQAPGQGLEWMGWISAYNGNTNYAQKFQGRV |
| | | | TMTTDTSTSTAYMELRSLRSDDTAVYYCAREVRLA |
| | | | ARPDHYYGMDVWGQGTTVTVSS |
| 131D12 | V_{H} 2 | 22 | QGQLVESGGGVVQPGRSLRLSCAASGFTFSSYGMH |
| | | | WVRQAPGKGLEWVAVIWHDGSNKYYADSVKGRFT |
| | | | ISRDNSKNTLYLQMNSLRAEDTAVYYCARVVPDNW |
| | | | NQDYYGMDVWGQGTTVTVSS |
| 27B2 | V_{H} 3 | 23 | QVQLVESGGGVVQPGRSLRLSCAASGFTFSSYGMH |
| | | | WVRQAPGKGLEWVAAIWYDGSNKYYAESVKGRFT |
| | | | ISRDNSKYTLYLQVNSLRAEDTAVYYCARRGGLAA |
| | | | RPGGLDVWGQGTTVTV |

TABLE 2C
Coding Sequence for Antibody Variable Light (V_{L}) Chains

| Contained | | SEQ ID | |
|---|---|---|---|
| in Clone | Designation | NO. | Coding Sequence |
| 101F7 | V_{L}1 | 24 | CAGTCTGTGTTGACGCAGCCGCCCTCAGTGTCTGCGG |
| | | | CCCCAGGACAGAAGGTCACCATCTCCTGCTCTGGAA |
| | | | GCAGCTCCAACATTGGCATTAATTATGTATCCTGGTA |
| | | | CCAACTACTCCCAGGAACAGCCCCCAAACTCCTCATT |
| | | | TATGACAATAATAAGCGACCCTTAGGGATTCCTGAC |
| | | | CGATTCTCTGGCTCCAAGTCTGGCACGTCAGCCACCC |
| | | | TGGGCATCACCGGACTCCAGACTGGGGACGAGACCG |
| | | | ATTATTACTGTGGAACATGGGATAGCAGCCTGAGTG |
| | | | CTTGGGTGTTCGGCGGAGGGACCAAGCTGACCGTCC |
| | | | TAGGT |
| 103B7 | V_{L}2 | 25 | CAGTCTGTGTTGACGCAGCCGCCCTCAGTGTCTGCGG |
| | | | CCCCAGGACAGAAGGTCACCATCTCCTGCTCTGGAA |
| | | | GTAGCTCCAACATTGGGATTAATTATGTATCCTGGTA |
| | | | CCAGCTACTCCCAGGAACAGCCCCCAAACTCCTCATT |
| | | | TATGACAATAATAAGCGACCCTTAGGGATTCCTGAC |
| | | | CGATTCTCTGGCTCCAAGTCTGGCACGTCAGTCACCC |
| | | | TGGGCATCACCGGACTCCAGACTGGGGACGAGGCCG |
| | | | ATTATTTCTGCGGAACATGGGATAGCAACCTGAGTG |
| | | | CTTGGGTTTTCGGCGGAGGGACCAAGCTGACCGTCC |
| | | | TAGGT |
| 131D12 | V_{L}3 | 26 | TCCTATGAGCTGACTCAGCCACCCTCAGTGTCCGTGT |
| | | | CCCCAGGACAGACGGCCAGCATCACCTGCTCTGGAG |
| | | | ATAAATTGGGGGATAAATATGCTTGCTGGTTTCAGC |
| | | | AGAAGCCAGGCCAGTCCCCTGTGCTGGTCATTTATCA |
| | | | AGATAGCAAGCGGCCCTTTGGGATCCCTGACCGATT |
| | | | CTCTGGCTCCAAGTCTGGGAACACAGCCACTCTGAC |
| | | | CATCAGCGGGACCCAGGCTATGGAAGAGGCTGACTA |
| | | | TTACTGTCAGGCGTGGGACAGTAGCACTGCGGTATT |
| | | | CGGCGGAGGGACCAAGCTGACCGTCCTAGGT |
| 27B2 | V_{L}4 | 27 | TCCTATGAGCTGACTCAGCCACCCTCAGTGTCCGTGT |
| | | | CCCCAGGACAGACAATCTCCATCAGCTGTTCTGGAG |
| | | | ATAAATTGGGGGATAAAAATGCTTGCTGGTATCAGC |
| | | | AGAAGCCAGGCCAGTCCCCTGGGCTGGTCATCTATC |
| | | | AGGATAGCAAGCGGCCCTTAGGGATCCCTGCGCGAT |
| | | | TCTCTGGCTCCAACTCTGGGAACACAGCCACTCTGAC |
| | | | CATCAGCGGGACCCAGGCTATGGATGAGGCTGACTA |
| | | | TTACTGTCAGGCGTGGGACATCAGCACTGTGGTATTC |
| | | | GGCGGAGGGACCAAGCTGACCGTCCTAGGT |

TABLE 2D
Coding Sequence for Antibody Variable Heavy (V_{H}) Chains

| Contained | | SEQ ID | |
|---|---|---|---|
| in Clone | Designation | NO. | Coding Sequence |
| 101F7 | V_{H} 1 | 28 | CAGGTTCAGCTGGTGCAGTCTGGAGCTGAGGTGAAG |
| | | | AAGCCTGGGGCCTCAGTGAAGGTCTCCTGCAAGGCC |
| | | | TCTGGTTACACCTTTAGCAGTTATGGTATCAGCTGGG |
| | | | TGCGACAGGCCCCTGGACAAGGGCTTGAGTGGATGG |
| | | | GATGGATCAGCGCTTACAATGGTAATACAAACTATG |
| | | | CACAGAAGTTCCAGGGCAGAGTCACCATGACCACAG |
| | | | ACACATCCACGAGCACAGCCTACATGGAGCTGAGGA |
| | | | GCCTGAGATCTGACGACACGGCCGTGTATTACTGTG |
| | | | CGAGAGAAGTTCGTCTGGCAGCTCGTCCTGACCATT |
| | | | ACTACGGTATGGACGTCTGGGGCCAAGGGACCACGG |
| | | | TCACCGTCTCCTCA |
| 103B7 | V_{H} 1 | 29 | CAGGTTCAGCTGGTGCAGTCTGGAGCTGAGGTGAAG |
| | | | AAGCCTGGGGCCTCAGTGAAGGTCTCCTGCAAGGCC |
| | | | TCTGGTTACACCTTTAGCAGTTATGGTATCAGCTGGG |
| | | | TGCGACAGGCCCCTGGACAAGGGCTTGAGTGGATGG |
| | | | GATGGATCAGCGCTTACAATGGTAATACAAACTATG |
| | | | CACAGAAGTTCCAGGGCAGAGTCACCATGACCACAG |
| | | | ACACATCCACGAGCACAGCCTACATGGAGCTGAGGA |
| | | | GCCTGAGATCTGACGACACGGCCGTGTATTACTGTG |
| | | | CGAGAGAAGTTCGTCTGGCAGCTCGTCCTGACCATT |
| | | | ACTACGGTATGGACGTCTGGGGCCAAGGGACCACGG |
| | | | TCACCGTCTCCTCA |
| 131D12 | V_{H} 2 | 30 | CAGGGGCAGCTGGTGGAGTCTGGGGGAGGCGTGGTC |
| | | | CAGCCTGGGAGGTCCCTGAGACTCTCCTGTGCAGCG |
| | | | TCTGGATTCACCTTCAGTAGCTATGGCATGCACTGGG |
| | | | TCCGCCAGGCTCCAGGCAAGGGGCTGGAGTGGGTGG |
| | | | CAGTCATATGGCATGATGGAAGTAATAAATACTATG |
| | | | CAGACTCCGTGAAGGGCCGATTCACCATCTCCAGAG |
| | | | ACAATTCCAAGAACACGCTGTATCTGCAAATGAACA |
| | | | GCCTGAGAGCCGAGGACACGGCTGTGTATTACTGTG |
| | | | CGAGAGTCGTTCCCGATAACTGGAACCAGGACTACT |
| | | | ACGGTATGGACGTCTGGGGCCAAGGGACCACGGTCA |
| | | | CCGTCTCCTCA |
| 27B2 | V_{H} 3 | 31 | CAGGTGCAGCTGGTGGAGTCTGGGGGAGGCGTGGTC |
| | | | CAGCCTGGGAGGTCCCTGAGACTCTCCTGTGCAGCG |
| | | | TCTGGATTCACCTTCAGTAGCTATGGCATGCACTGGG |
| | | | TCCGCCAGGCTCCAGGCAAGGGGCTGGAGTGGGTGG |
| | | | CAGCTATATGGTATGATGGAAGTAATAAATACTATG |
| | | | CAGAGTCCGTGAAGGGCCGATTCACCATCTCCAGAG |
| | | | ACAATTCCAAGTACACGCTGTATCTGCAAGTGAACA |
| | | | GCCTGAGAGCCGAGGACACGGCTGTGTATTACTGTG |
| | | | CGAGGAGGGGGGGTCTGGCAGCTCGTCCGGGCGGTC |
| | | | TGGACGTCTGGGGCCAAGGGACCACGGTCACCGTCT |
| | | | CCTCA |

Each of the heavy chain variable regions listed in Table 2B can be combined with any of the light chain variable regions shown in Table 2A to form an antigen binding protein. Examples of such combinations include V_{H}1 combined with any of V_{L}1, V_{L}2, V_{L}3 or V_{L}4; V_{H}2 combined with any of V_{L}1, V_{L}2, V_{L}3 or V_{L}4; V_{H}3 combined with any of V_{L}1, V_{L}2, V_{L}3, or V_{L}4, and so on. In embodiments, the antigen binding proteins of the invention comprise V_{H}1 combined with V_{L}1, V_{H}1 combined with V_{L}2, V_{H}2 combined with V_{L}3 and V_{H}3 combined with V_{L}4.

In some instances, the antigen binding protein includes at least one heavy chain variable region and/or one light chain variable region from those listed in Tables 2A and 2B. In some instances, the antigen binding protein includes at least two different heavy chain variable regions and/or light chain variable regions from those listed in Table 2B. An example of such an antigen binding protein comprises (a) one V_{H}1, and (b) one of V_{H}2, V_{H}3, etc. Still another example of such an antigen binding protein comprises (a) one V_{L}1, and (b) one of V_{L}2 or V_{L}3. Again another example of such an antigen binding protein comprises (a) one V_{L}2, and (b) one of V_{L}1 or V_{L}3. Again another example of such an antigen binding protein comprises (a) one V_{L}3, and (b) one of V_{L}1 or V_{L}2 etc.

The various combinations of heavy chain variable regions can be combined with any of the various combinations of light chain variable regions.

In other embodiments, an antigen binding protein comprises two identical light chain variable regions and/or two identical heavy chain variable regions. As an example, the antigen binding protein can be an antibody or immunologically functional fragment thereof that includes two light chain variable regions and two heavy chain variable regions in combinations of pairs of light chain variable regions and pairs of heavy chain variable regions as listed in Tables 2A and 2B.

Some antigen binding proteins that are provided comprise a heavy chain variable domain comprising a sequence of amino acids that differs from the sequence of a heavy chain variable domain selected from V_{H}1 or V_{H}2, at only 1, 2, 3, 4, 5, 6, 7, 8, 9, 10, 11, 12, 13, 14 or 15 amino acid residues, wherein each such sequence difference is independently either a deletion, insertion or substitution of one amino acid, with the deletions, insertions and/or substitutions resulting in no more than 15 amino acid changes relative to the foregoing variable domain sequences. The heavy chain variable region in some antigen binding proteins comprises a sequence of amino acids that has at least 70%, 75%, 80%, 85%, 90%, 95%, 97% or 99% sequence identity to the amino acid sequences of the heavy chain variable region of V_{H}1 or V_{H}2.

Certain antigen binding proteins comprise a light chain variable domain comprising a sequence of amino acids that differs from the sequence of a light chain variable domain selected from V_{L}1, V_{L}2 or V_{L}3, at only 1, 2, 3, 4, 5, 6, 7, 8, 9, 10, 11, 12, 13, 14 or 15 amino acid residues, wherein each such sequence difference is independently either a deletion, insertion or substitution of one amino acid, with the deletions, insertions and/or substitutions resulting in no more than 15 amino acid changes relative to the foregoing variable domain sequences. The light chain variable region in some antigen binding proteins comprises a sequence of amino acids that has at least 70%, 75%, 80%, 85%, 90%, 95%, 97% or 99% sequence identity to the amino acid sequences of the light chain variable region of V_{L}1, V_{L}2, or V_{L}3.

In additional instances, antigen binding proteins comprise the following pairings of light chain and heavy chain variable domains: V_{L}1 with V_{H}1, V_{L}2 with V_{H}1, V_{L}3 with V_{H}1, V_{L}1 with V_{H} 2, V_{L}2 with V_{H}2 or, V_{L}3 with V_{H}2,

In some instances, the antigen binding proteins in the above pairings can comprise amino acid sequences that have at least 70%, 75%, 80%, 85%, 90%, 95%, 96%, 97%, 98% or 99% sequence identity with the specified heavy chain variable domain and at least 70%, 75%, 80%, 85%, 90%, 95%, 96%, 97%, 98% or 99% sequence identity with the specified light chain variable domains. In a particular embodiment the heavy chain variable domain and the light chain variable domain in the above pairings each comprise at least 70%, 75%, 80%, 85%, 90%, 95%, 96%, 97%, 98% or 99% sequence identity with the specified variable heavy chain domain and light chain variable domain, respectively.

Still other antigen binding proteins, e.g., antibodies or immunologically functional fragments, include variant forms of a variant heavy chain and a variant light chain as just described.

### Antigen Binding Protein CDRs

In various embodiments, the antigen binding proteins disclosed herein can comprise polypeptides into which one or more CDRs are grafted, inserted and/or joined. An antigen binding protein can have 1, 2, 3, 4, 5 or 6 CDRs. An antigen binding protein thus can have, for example, one heavy chain CDR1 (“CDRH1”), and/or one heavy chain CDR2 (“CDRH2”), and/or one heavy chain CDR3 (“CDRH3”), and/or one light chain CDR1 (“CDRL1”), and/or one light chain CDR2 (“CDRL2”), and/or one light chain CDR3 (“CDRL3”). Some antigen binding proteins include both a CDRH3 and a CDRL3. Specific heavy and light chain CDRs are identified in Tables 3A and 3B, respectively, infra.

Complementarity determining regions (CDRs) and framework regions (FR) of a given antibody, as used herein, are specified by the system described by Kabat et al., (1991) “Sequences of Proteins of Immunological Interest”, 5^{th} Ed., US Dept. of Health and Human Services, PHS, NIH, NIH Publication no. 91-3242. Certain antibodies that are disclosed herein comprise one or more amino acid sequences that are identical or have substantial sequence identity to the amino acid sequences of one or more of the CDRs presented in Table 3A (CDRHs) and Table 3B (CDRLs), infra.

TABLE 3A
Exemplary CDRH Sequences

| | SEQ | Contained | | |
|---|---|---|---|---|
| | ID | in | | |
| Clone | NO: | Reference | Designation | Sequence |
| 101F7 | 34 | V_{H}1 | CDRH1-1 | SYGIS |
| 103B7 | | V_{H}1 | | |
| 131D12 | 35 | V_{H}2 | CDRH1-2 | SYGMH |
| 101F7 | 36 | V_{H}1 | CDRH2-1 | WISAYNGNTNYAQKFQG |
| 103B7 | | V_{H}1 | | |
| 131D12 | 37 | V_{H}2 | CDRH2-2 | VIWHDGSNKYYADSVKG |
| 101F7 | 38 | V_{H}1 | CDRH3-1 | EVRLAARPDHYYGMDV |
| 103B7 | | V_{H}1 | | |
| 131D12 | 39 | V_{H}2 | CDRH3-2 | VVPDNWNQDYYGMDV |
| 27B2.6 | 40 | V_{H}3 | CDRH1-3 | SYGMH |
| 27B2.6 | 41 | V_{H}3 | CDRH2-3 | AIWYDGSNKYYAESVKG |
| 27B2.6 | 42 | V_{H}3 | CDRH3-3 | RGGLAARPGGLDV |

TABLE 3B
Exemplary CDRL Sequences

| | SEQ | | | |
|---|---|---|---|---|
| | ID | Contained in | | Amino |
| Clone | NO: | Reference | Designation | Acid Sequence |
| 101F7 | 43 | V_{L}1 | CDRL1-1 | SGSSSNIGINYVS |
| 103B7 | | V_{L}2 | | |
| 131D12 | 44 | V_{L}3 | CDRL1-2 | SGDKLGDKYAC |
| 101F7 | 45 | V_{L}1 | CDRL2-1 | DNNKRPL |
| 103B7 | | V_{L}2 | | |
| 131D12 | 46 | V_{L}3 | CDRL2-2 | QDSKRPF |
| 101F7 | 47 | V_{L}1 | CDRL3-1 | GTWDSSLSAWV |
| 103B7 | 48 | V_{L}2 | CDRL3-2 | GTWDSNLSAWV |
| 131D12 | 49 | V_{L}3 | CDRL3-3 | QAWDSSTAV |
| 27B2 | 50 | V_{L}3 | CDRL1-3 | SGDKLGDKNAC |
| 27B2 | 51 | V_{L}3 | CDRL2-3 | QDSKRPL |
| 27B2 | 52 | V_{L}3 | CDRL3-3 | QAWDISTVV |

TABLE 3C
Coding Sequences for CDRHs

| | SEQ | Contained | | |
|---|---|---|---|---|
| | ID | in | | |
| Clone | NO: | Reference | Designation | Coding Sequences |
| 101F7 | 53 | V_{H}1 | CDRH1-1 | AGTTATGGTATCAGC |
| 103B7 | | V_{H}1 | | |
| 131D12 | 54 | V_{H}2 | CDRH1-2 | AGCTATGGCATGCAC |
| 101F7 | 55 | V_{H}1 | CDRH2-1 | TGGATCAGCGCTTACAATGGTAATACAAACT |
| 103B7 | | V_{H}1 | | ATGCACAGAAGTTCCAGGGC |
| 131D12 | 56 | V_{H}2 | CDRH2-2 | GTCATATGGCATGATGGAAGTAATAAATACT |
| | | | | ATGCAGACTCCGTGAAGGGC |
| 101F7 | 57 | V_{H}1 | CDRH3-1 | GAAGTTCGTCTGGCAGCTCGTCCTGACCATT |
| 103B7 | | V_{H}1 | | ACTACGGTATGGACGTC |
| 131D12 | 58 | V_{H}2 | CDRH3-2 | GTCGTTCCCGATAACTGGAACCAGGACTACT |
| | | | | ACGGTATGGACGTC |
| 27B2 | 59 | V_{H}3 | CDRH1-3 | AGCTATGGCATGCAC |
| 27B2 | 60 | V_{H}3 | CDRH2-3 | GCTATATGGTATGATGGAAGTAATAAATACT |
| | | | | ATGCAGAGTCCGTGAAGGGC |
| 27B2 | 61 | V_{H}3 | CDRH3-3 | AGGGGGGGTCTGGCAGCTCGTCCGGGCGGTC |
| | | | | TGGACGTC |

TABLE 3D
Coding Sequences for CDRLs

| | SEQ | Contained | | |
|---|---|---|---|---|
| | ID | in | | |
| Clone | NO: | Reference | Designation | Coding Sequence |
| 101F7 | 62 | V_{L}1 | CDRL1-1 | TCTGGAAGCAGCTCCAACATTGGCATTA |
| 103B7 | | | | ATTATGTATCC |
| 131D12 | 63 | V_{L}3 | CDRL1-2 | TCTGGAGATAAATTGGGGGATAAATATG |
| | | | | CTTGC |
| 101F7 | 64 | V_{L}1 | CDRL2-1 | GACAATAATAAGCGACCCTTA |
| 103B7 | | | | |
| 131D12 | 65 | V_{L}3 | CDRL2-2 | CAAGATAGCAAGCGGCCCTTT |
| 101F7 | 66 | V_{L}1 | CDRL3-1 | GGAACATGGGATAGCAGCCTGAGTGCTT |
| | | | | GGGTG |
| 103B7 | 67 | V_{L}2 | CDRL3-2 | GGAACATGGGATAGCAACCTGAGTGCTT |
| | | | | GGGTT |
| 131D12 | 68 | V_{L}3 | CDRL3-3 | CAGGCGTGGGACAGTAGCACTGCGGTA |
| 27B2 | 69 | V_{L}3 | CDRL1-3 | TCTGGAGATAAATTGGGGGATAAAAAT |
| | | | | GCTTGC |
| 27B2 | 70 | V_{L}3 | CDRL2-3 | CAGGATAGCAAGCGGCCCTTA |
| 27B2 | 71 | V_{L}3 | CDRL3-3 | CAGGCGTGGGACATCAGCACTGTGGTA |

The structure and properties of CDRs within a naturally occurring antibody has been described, supra. Briefly, in a traditional antibody, the CDRs are embedded within a framework in the heavy and light chain variable region where they constitute the regions responsible for antigen binding and recognition. A variable region comprises at least three heavy or light chain CDRs, see, e.g., Kabat et al., (1991) “Sequences of Proteins of Immunological Interest”, 5^{th} Ed., US Dept. of Health and Human Services, PHS, NIH, NIH Publication no. 91-3242; see also Chothia and Lesk, (1987) J. Mol. Biol. 196:901-917; Chothia et al., (1989) Nature 342: 877-883), within a framework region (designated framework regions 1-4, FR1, FR2, FR3, and FR4, by Kabat et al., (1991); see also Chothia and Lesk, (1987) supra). The CDRs provided herein, however, can not only be used to define the antigen binding domain of a traditional antibody structure, but can be embedded in a variety of other polypeptide structures, as described herein.

In one aspect, the CDRs provided are (a) a CDRH selected from the group consisting of (i) a CDRH1 selected from the group consisting of SEQ ID NOS 12-15, 20-23, 34, 35, and 40; (ii) a CDRH2 selected from the group consisting of SEQ ID NOS 12-15, 20-23, 36, 37; and 41 (iii) a CDRH3 selected from the group consisting of SEQ ID NOS 12-15, 20-23, 38, 39; and 42; and (iv) a CDRH of (i), (ii) and (iii) that contains one or more amino acid substitutions, deletions or insertions of no more than five, four, three, two, or one amino acids; (B) a CDRL selected from the group consisting of (i) a CDRL1 selected from the group consisting of SEQ ID NOS 8-11, 16-19; 43, 44 and 50 (ii) a CDRL2 selected from the group consisting of SEQ ID NOS 8-11, 16-19; 45, 46 and 51; (iii) a CDRL3 selected from the group consisting of SEQ ID NOS 8-11, 16-19; 47, 48 and 52; and (iv) a CDRL of (i), (ii) and (iii) that contains one or more amino acid substitutions, deletions or insertions of no more than 1, 2, 3, 4, or 5 amino acids amino acids.

In another aspect, an antigen binding protein comprises 1, 2, 3, 4, 5, or 6 variant forms of the CDRs listed in Tables 3A and 3B, infra, each having at least 80%, 85%, 90%, 95%, 96%, 97%, 98%, or 99% sequence identity to a CDR sequence listed in Tables 3A and 3B, infra. Some antigen binding proteins comprise 1, 2, 3, 4, 5, or 6 of the CDRs listed in Tables 3A and 3B, infra, each differing by no more than 1, 2, 3, 4 or 5 amino acids from the CDRs listed in these tables.

In still another aspect, an antigen binding protein includes the following associations of CDRL1, CDRL2 and CDRL3, presented for convenience in tabular form and in reference to the clone source of the association:

TABLE 4
CDRL Associations

| Clone ID | CDRL1 | CDRL2 | CDRL3 |
|---|---|---|---|
| 101F7 | CDRL1-1 | CDRL2-1 | CDRL3-1 |
| 103B7 | CDRL1-1 | CDRL2-1 | CDRL3-2 |
| 131D12 | CDRL1-2 | CDRL2-2 | CDRL3-3 |

In an additional aspect, an antigen binding protein includes the following associations of CDRH1, CDRH2 and CDRH3, presented for convenience in tablular form and in reference to the clone source of the association:

TABLE 5
CDRH Associations

| Clone ID | CDRH1 | CDRH2 | CDRH3 |
|---|---|---|---|
| 101F7 | CDRH1-1 | CDRH2-1 | CDRH3-1 |
| 103B7 | CDRH1-1 | CDRH2-1 | CDRH3-1 |
| 131D12 | CDRH1-2 | CDRH2-2 | CDRH3-2 |

In an additional aspect, an antigen binding protein includes the following associations of CDRH1, CDRH2, CDRH3, CDRL1, CDRL2 and CDRL3 presented for convenience in tablular form and in reference to the clone source of the association:

TABLE 6
CDRH and CDRL Associations

| Clone ID | CDRL1 | CDRL2 | CDRL3 | CDRH1 | CDRH2 | CDRH3 |
|---|---|---|---|---|---|---|
| 101F7 | CDRL1-1 | CDRL2-1 | CDRL3-1 | CDRH1-1 | CDRH2-1 | CDRH3-1 |
| 103B7 | CDRL1-1 | CDRL2-1 | CDRL3-2 | CDRH1-1 | CDRH2-1 | CDRH3-1 |
| 131D12 | CDRL1-2 | CDRL2-2 | CDRL3-3 | CDRH1-2 | CDRH2-2 | CDRH3-2 |

### Exemplary Antigen Binding Proteins

According to one aspect, an isolated antigen binding protein comprising (a) one or more heavy chain complementary determining regions (CDRHs) comprising one or more of: (i) a CDRH1 selected from the group consisting of SEQ ID NOS 12-15, 20-23, 34, 35, and 40; (ii) a CDRH2 selected from the group consisting of SEQ ID NOS 12-15, 20-23, 36, 37; and 41 (iii) a CDRH3 selected from the group consisting of SEQ ID NOS 12-15, 20-23, 38, 39; and 42; and (iv) a CDRH of (i), (ii) and (iii) that comprises ten, nine, eight, seven, six, five, four, three, two or one amino acid substitutions, deletions, insertions and combinations thereof; (b) one or more light chain complementary determining regions (CDRLs) comprising one or more of: (i)) a CDRL selected from the group consisting of (i) a CDRL1 selected from the group consisting of SEQ ID NOS 8-11, 16-19; 43, 44 and 50 (ii) a CDRL2 selected from the group consisting of SEQ ID NOS 8-11, 16-19; 45, 46 and 51; (iii) a CDRL3 selected from the group consisting of SEQ ID NOS 8-11, 16-19; 47, 48 and 52

and (iv) a CDRL of (i), (ii) and (iii) that comprises ten, nine, eight, seven, six, five, four, three, four, two or one amino acid substitutions, deletions or insertions and combinations thereof; or (c) one or more heavy chain CDRHs of (a) and one or more light chain CDRLs of (b).

In another embodiment, the CDRHs have at least 70%, 75%, 80%, 85%, 90%, 95%, 96%, 97%, 98% or 99% sequence identity with an amino acid sequence selected from the group consisting of SEQ ID NOS 12-15, and/or the CDRLs have at least 70%, 75%, 80%, 85%, 90%, 95%, 96%, 97%, 98% or 99% sequence identity with an amino acid sequence selected from the group consisting of SEQ ID NOS 8-11. In a further embodiment, the VH is selected from the group consisting of SEQ ID NOS 12-15, and/or the V_{L} is selected from the group consisting of SEQ ID NOS 8-11.

According to one aspect, an isolated antigen binding protein comprising (a) one or more variable heavy chains (V_{H}S) comprising one or more of: (i) SEQ ID NOS 12-15; and (ii) a V_{H} of (i) that comprises ten, nine, eight, seven, six, five, four, three, two or one amino acid substitutions, deletions, insertions and combinations thereof; (b) one or more variable light chains (V_{L}S) selected from the group consisting of: (i) SEQ ID NOS 8-11, and (ii) a V_{L} of (i) that comprises ten, nine, eight, seven, six, five, four, three, two or one amino acid substitutions, deletions, insertions and combinations thereof; or (c) one or more variable heavy chains of (a) and one or more variable light chains of (b).

In another embodiment, the variable heavy chain (V_{H}) has at least 70%, 75%, 80%, 85%, 90%, 95%, 96%, 97%, 98% or 99% sequence identity with an amino acid sequence selected from the group consisting of SEQ ID NOS 12-15, and/or the variable light chain (V_{L}) has at least 70%, 75%, 80%, 85%, 90%, 95%, 96%, 97%. 98% or 99% sequence identity with an amino acid sequence selected from the group consisting of SEQ ID NOS 8-11.

In one aspect, also provided is an antigen binding protein that specifically binds to a linear or three-dimensional epitope comprising one or more amino acid residues from PCSK9, particularly human PCSK9.

In yet another embodiment, the isolated antigen binding protein described hereinabove comprises a first amino acid sequence comprising at least one of the CDRH consensus sequences disclosed herein, and a second amino acid sequence comprising at least one of the CDRL consensus sequences disclosed herein.

In one aspect, the first amino acid sequence comprises at least two of the CDRH consensus sequences, and/or the second amino acid sequence comprises at least two of the CDRL consensus sequences. In certain embodiments, the first and the second amino acid sequence are covalently bonded to each other.

In a further embodiment, the first amino acid sequence of the isolated antigen binding protein comprises the CDRH3, the CDRH2 and the CDRH1 parings shown in 6 for each clone, and/or the second amino acid sequence of the isolated antigen binding protein comprises the CDRL3, the CDRL2 and the CDRL1 pairings shown in Table 4 for each clone.

In a further embodiment, the antigen binding protein comprises at least two CDRH sequences of heavy chain sequences CDRH1-1, CDRH2-1, CDRH3-1, CDRh1-2, CDRH2-2 and CDRH3-2 as shown in table 3A.

In again a further embodiment, the antigen binding protein comprises at least two CDRL sequences of light chain sequences CDRL1-1, CDRL2-1, CDRL3-1, CDRL1-2, CDRL2-2 and CDRL3-2 and CDRL3-3, as shown in Table 3B.

In still a further embodiment, the antigen binding protein comprises at least two CDRH sequences of heavy chain sequences CDRH1-1, CDRH2-1, CDRH3-1, CDRh1-2, CDRH2-2 and CDRH3-2 as shown in table 3A. and at least two CDRLs of light chain sequences CDRL1-1, CDRL2-1, CDRL3-1, CDRL1-2, CDRL2-2 and CDRL3-2 and CDRL3-3, as shown in Table 3B.

In again another embodiment, the antigen binding protein comprises the CDRH1, CDRH2, and CDRH3 sequences of heavy chain sequences H1, H2, and H3 as shown in Tables 3A.

In yet another embodiment, the antigen binding protein comprises the CDRL1, CDRL2, and CDRL3 sequences of light chain sequences L1, L2 and L3 as shown in Table 3B.

In yet another embodiment, the antigen binding protein comprises all six CDRs of an antigen binding protein comprising the following V_{H} and V_{L} pairs: V_{L}1 with V_{H}1; V_{L}2 with V_{H}1; V_{L}3 with V_{H}1, V_{L}1 with V_{H}2; V_{L}2 with V_{H}2; and V_{L}3 with V_{H}2 as shown in Tables 2A and 2B and Table 6.

In one aspect, the isolated antigen binding proteins that specifically bind to PCSK9 provided herein can be a monoclonal antibody, a polyclonal antibody, a recombinant antibody, a human antibody, a humanized antibody, a chimeric antibody, a multispecific antibody, or an antibody fragment thereof.

In another embodiment, the antibody fragment of the isolated antigen-binding proteins provided herein can be a Fab fragment, a Fab′ fragment, an F(ab′)₂ fragment, an Fv fragment, a diabody, or a single chain antibody molecule.

In a further embodiment, an isolated antigen binding protein that specifically binds to PCSK9 provided herein is a human antibody and can be of the IgG1-, IgG2- IgG3- or IgG4-type.

In another embodiment, an isolated antigen binding protein that specifically binds to PCSK9 comprises a light or a heavy chain polypeptide as set forth in Tables 1A-1B. In some embodiments, an antigen binding protein that specifically binds to PCSK9 comprises a variable light or variable heavy domain such as those listed in Tables 2A-2B. In still other embodiments, an antigen binding protein that specifically binds to PCSK9 comprises one, two or three CDRHs or one, two or three CDRLs as set forth in Tables 3A-3B, 4A-4B, infra. In still other embodiments, an antigen binding protein that specifically binds to PCSK9 comprises all six CDRS as shown in Table 6. Such antigen binding proteins, and indeed any of the antigen binding proteins disclosed herein, can be PEGylated with one or more PEG molecules, for examples PEG molecules having a molecular weight selected from the group consisting of 5K, 10K, 20K, 40K, 50K, 60K, 80K, 100K or greater than 100K.

In yet another aspect, any antigen binding protein that specifically binds to PCSK9 provided herein can be coupled to a labeling group and can compete for binding to PCSK9 with an antigen binding protein of one of the isolated antigen binding proteins provided herein. In one embodiment, the isolated antigen binding protein provided herein can decrease blood triglyceride and cholesterol levels or improve other cardiovascular risk factors when administered to a patient, such as decrease blood total cholesterol, LDL-C, VLDL-C, apolipoprotein B, non-HDL-C, lipoprotein (a), and increase HDL-C.

As will be appreciated, for any antigen binding protein comprising more than one CDR provided in Tables 3A-3B, and any combination of CDRs independently selected from the depicted sequences may be useful. Thus, antigen binding proteins with one, two, three, four, five or six of independently selected CDRs can be generated. However, as will be appreciated by those in the art, specific embodiments generally utilize combinations of CDRs that are non-repetitive, e.g., antigen binding proteins are generally not made with two CDRH2 regions, etc.

Some of the antigen binding proteins that specifically bind to PCSK9 that are provided herein are discussed in more detail below.

### Antigen Binding Proteins and Binding Epitopes and Binding Domains

When an antigen binding protein is said to bind an epitope on PCSK9, what is meant is that the antigen binding protein specifically binds to a specified portion of PCSK9. In some embodiments, the antigen binding protein can specifically bind to a polypeptide consisting of specified residues (e.g., a specified segment of PCSK9).

In any of the foregoing embodiments, such an antigen binding protein does not need to contact every residue of PCSK9. Nor does every single amino acid substitution or deletion within PCSK9, necessarily significantly affect binding affinity.

Epitope specificity and the binding domain(s) of an antigen binding protein can be determined by a variety of methods. Some methods, for example, can use truncated portions of an antigen. Other methods utilize antigen mutated at one or more specific residues, such as by employing an alanine scanning or arginine scanning-type approach or by the generation and study of chimeric proteins in which various domains, regions or amino acids are swapped between two proteins (e.g., mouse and human forms of one or more of the antigens or target proteins), or by protease protection assays.

### Competing Antigen Binding Proteins

In another aspect, antigen binding proteins are provided that compete with one of the exemplified antibodies or functional fragments for binding PCSK9. Such antigen binding proteins can also bind to the same epitope as one of the herein exemplified antigen binding proteins, or an overlapping epitope. Antigen binding proteins and fragments that compete with or bind to the same epitope as the exemplified antigen binding proteins are expected to show similar functional properties. The exemplified antigen binding proteins and fragments include those with the heavy and light chains H1-H3 and L1-L4, variable region domains V_{L}1-V_{L}4 and V_{H}1-V_{H3}, and CDRs provided herein, including those in Tables 1, 2, 3, 4, 5 and 6. Thus, as a specific example, the antigen binding proteins that are provided include those that compete with an antibody comprising:

(a) 1, 2, 3, 4, 5 or all 6 of the CDRs listed for an antigen binding protein listed in Tables 3A and 3B, infra;

(b) a V_{H} and a V_{L} selected from V_{L}1-V_{L3} and V_{H}1-V_{H}2 and listed for an antigen binding protein listed in Tables 2A and 2B; or

(c) two light chains and two heavy chains as specified for an antigen binding protein listed in Tables 1A and 1B, infra.

Thus, in one embodiment, the present disclosure provides antigen binding proteins, including human antibodies, that competes for binding to PCSK9 with a reference antibody, wherein the reference antibody comprises a combination of light chain and heavy chain variable domain sequences selected from the group consisting of V_{L}1 with V_{H}1; V_{L}2 with V_{H}1; V_{L}3 with V_{H}1, V_{L}1 with V_{H}2; V_{L}2 with V_{H}2; and V_{L}3 with V_{H}2.

In another embodiment, the present disclosure provides antigen binding proteins, including human antibodies, that compete for PCSK9 with a reference antibody, wherein the reference antibody is 101F7, 103B7, 131D12 or 27B2.

In a further embodiment, an isolated antigen binding protein, such as a human antibody, is provided that binds to PCSK9 with substantially the same Kd as a reference antibody; reduces the ability of PCSK9 to block LDL uptake in vitro in a human HepG2 cell assay (or other suitable cell line or primary cell in culture) to the same degree as a reference antibody; lowers serum cholesterol levels; and/or competes for binding with said reference antibody to PCSK9, wherein the reference antibody is selected from the group consisting of 101F7, 103B7, 131D12 and 27B2. The ability to compete with an antibody can be determined using any suitable assay, such as those described herein, in which antigen binding proteins 101F7, 103B7, 131D12 and 27B2 can be used as the reference antibody.

### Monoclonal Antibodies

The antigen binding proteins that are provided include monoclonal antibodies that bind to PCSK9, and inhibit PCSK9 binding to LDLR to various degrees. Monoclonal antibodies can be produced using any technique known in the art, e.g., by immortalizing spleen cells harvested from the transgenic animal after completion of the immunization schedule. The spleen cells can be immortalized using any technique known in the art, e.g., by fusing them with myeloma cells to produce hybridomas. Myeloma cells for use in hybridoma-producing fusion procedures preferably are non-antibody-producing, have high fusion efficiency, and enzyme deficiencies that render them incapable of growing in certain selective media which support the growth of only the desired fused cells (hybridomas). Examples of suitable cell lines for use in mouse fusions include Sp-20, P3-X63/Ag8, P3-X63-Ag8.653, NS1/1.Ag 4 1, Sp210-Ag14, FO, NSO/U, MPC-11, MPC11-X45-GTG 1.7 and S194/5XXO Bul; examples of cell lines used in rat fusions include R210.RCY3, Y3-Ag 1.2.3, IR983F and 4B210. Other cell lines useful for cell fusions are U-266, GM1500-GRG2, LICR-LON-HMy2 and UC729-6.

In some instances, a hybridoma cell line is produced by immunizing an animal (e.g., a transgenic animal having human immunoglobulin sequences) with an immunogen comprising (1) self-cleaved, mature, secreted PCSK9 comprising amino acids 31 to 692 of the amino acid sequence of SEQ ID NO: 2 (as shown in Example 1); harvesting spleen cells from the immunized animal; fusing the harvested spleen cells to a myeloma cell line, thereby generating hybridoma cells; establishing hybridoma cell lines from the hybridoma cells (as shown in Example 2), and identifying a hybridoma cell line that produces an antibody that binds to PCSK9 and blocks PCSK9 from binding to LDLR (e.g., as described in Example 3). Such hybridoma cell lines, and the monoclonal antibodies produced by them, form aspects of the present disclosure.

Monoclonal antibodies secreted by a hybridoma cell line can be purified using any technique known in the art. Hybridomas or mAbs can be further screened to identify mAbs with particular properties, such as the ability to block PCSK9 from binding to LDLR. Examples of such screens are provided herein.

### Chimeric and Humanized Antibodies

Chimeric and humanized antibodies based upon the foregoing sequences can readily be generated. One example is a chimeric antibody, which is an antibody composed of protein segments from different antibodies that are covalently joined to produce functional immunoglobulin light or heavy chains or immunologically functional portions thereof. Generally, a portion of the heavy chain and/or light chain is identical with or homologous to a corresponding sequence in antibodies derived from a particular species or belonging to a particular antibody class or subclass, while the remainder of the chain(s) is/are identical with or homologous to a corresponding sequence in antibodies derived from another species or belonging to another antibody class or subclass. For methods relating to chimeric antibodies, see, for example, U.S. Pat. No. 4,816,567; and Morrison et al., (1985) Proc. Natl. Acad. Sci. USA 81:6851-6855, which are hereby incorporated by reference. CDR grafting is described, for example, in U.S. Pat. No. 6,180,370, No. 5,693,762, No. 5,693,761, No. 5,585,089, and No. 5,530,101.

Generally, a goal of making a chimeric antibody is to create a chimera in which the number of amino acids from the intended patient/recipient species is maximized. One example is the “CDR-grafted” antibody, in which the antibody comprises one or more complementarity determining regions (CDRs) from a particular species or belonging to a particular antibody class or subclass, while the remainder of the antibody chain(s) is/are identical with or homologous to a corresponding sequence in antibodies derived from another species or belonging to another antibody class or subclass. For use in humans, the variable region or selected CDRs from a rodent antibody often are grafted into a human antibody, replacing the naturally-occurring variable regions or CDRs of the human antibody.

One useful type of chimeric antibody is a “humanized” antibody. Generally, a humanized antibody is produced from a monoclonal antibody raised initially in a non-human animal. Certain amino acid residues in this monoclonal antibody, typically from non-antigen recognizing portions of the antibody, are modified to be homologous to corresponding residues in a human antibody of corresponding isotype. Humanization can be performed, for example, using various methods by substituting at least a portion of a rodent variable region for the corresponding regions of a human antibody (see, e.g., U.S. Pat. No. 5,585,089, and U.S. Pat. No. 5,693,762; Jones et al., (1986) Nature 321:522-525; Riechmann et al., (1988) Nature 332:323-27; Verhoeyen et al., (1988) Science 239:1534-1536).

In one aspect, the CDRs of the light and heavy chain variable regions of the antibodies provided herein (e.g., in Tables 3-6) are grafted to framework regions (FRs) from antibodies from the same, or a different, phylogenetic species. For example, the CDRs of the heavy and light chain variable regions V_{H}1, V_{H}2, and/or V_{L}1, V_{L}2, V_{L}3, can be grafted to consensus human FRs. To create consensus human FRs, FRs from several human heavy chain or light chain amino acid sequences can be aligned to identify a consensus amino acid sequence. In other embodiments, the FRs of a heavy chain or light chain disclosed herein are replaced with the frameworks (“FRs”) from a different heavy chain or light chain. In one aspect, rare amino acids in the FRs of the heavy and light chains of an antigen binding protein (e.g., an antibody) that specifically binds to a PCSK9 are not replaced, while the rest of the FR amino acids are replaced. A “rare amino acid” is a specific amino acid that is in a position in which this particular amino acid is not usually found in an FR. Alternatively, the grafted variable regions from the one heavy or light chain can be used with a constant region that is different from the constant region of that particular heavy or light chain as disclosed herein. In other embodiments, the grafted variable regions are part of a single chain Fv antibody.

In certain embodiments, constant regions from species other than human can be used along with the human variable region(s) to produce hybrid antibodies.

### Fully Human Antibodies

Fully human antibodies are provided by the instant disclosure. Methods are available for making fully human antibodies specific for a given antigen without exposing human beings to the antigen (“fully human antibodies”). One specific means provided for implementing the production of fully human antibodies is the “humanization” of the mouse humoral immune system. Introduction of human immunoglobulin (Ig) loci into mice in which the endogenous Ig genes have been inactivated is one means of producing fully human monoclonal antibodies (mAbs) in mouse, an animal that can be immunized with any desirable antigen. Using fully human antibodies can minimize the immunogenic and allergic responses that can sometimes be caused by administering mouse or mouse-derived mAbs to humans as therapeutic agents.

Fully human antibodies can be produced by immunizing transgenic animals (typically mice) that are capable of producing a repertoire of human antibodies in the absence of endogenous immunoglobulin production. Antigens for this purpose typically have six or more contiguous amino acids, and optionally are conjugated to a carrier, such as a hapten. See, e.g., Jakobovits et al., (1993) Proc. Natl. Acad. Sci. USA 90:2551-2555; Jakobovits et al., (1993) Nature 362:255-258; and Bruggermann et al., (1993) Year in Immunol. 7:33. In one example of such a method, transgenic animals are produced by incapacitating the endogenous mouse immunoglobulin loci encoding the mouse heavy and light immunoglobulin chains therein, and inserting into the mouse genome large fragments of human genome DNA containing loci that encode human heavy and light chain proteins. Partially modified animals, which have less than the full complement of human immunoglobulin loci, are then cross-bred to obtain an animal having all of the desired immune system modifications. When administered an immunogen, these transgenic animals produce antibodies that are immunospecific for the immunogen but have human rather than murine amino acid sequences, including the variable regions. For further details of such methods, see, e.g., WO96/33735 and WO94/02602. Additional methods relating to transgenic mice for making human antibodies are described in U.S. Pat. No. 5,545,807; U.S. Pat. No. 6,713,610; U.S. Pat. No. 6,673,986; U.S. Pat. No. 6,162,963; U.S. Pat. No. 5,545,807; U.S. Pat. No. 6,300,129; U.S. Pat. No. 6,255,458; U.S. Pat. No. 5,877,397; U.S. Pat. No. 5,874,299 and U.S. Pat. No. 5,545,806; in PCT publications WO91/10741, WO90/04036, and in EP 546073 and EP 546073.

According to certain embodiments, antibodies of the invention can be prepared through the utilization of a transgenic mouse that has a substantial portion of the human antibody producing genome inserted but that is rendered deficient in the production of endogenous, murine antibodies. Such mice, then, are capable of producing human immunoglobulin molecules and antibodies and are deficient in the production of murine immunoglobulin molecules and antibodies. Technologies utilized for achieving this result are disclosed in the patents, applications and references disclosed in the specification, herein. In certain embodiments, one can employ methods such as those disclosed in PCT Published Application No. WO 98/24893 or in Mendez et al., (1997) Nature Genetics, 15:146-156, which are hereby incorporated by reference for any purpose.

Generally, fully human monoclonal antibodies specific for PCSK9 can be produced as follows. Transgenic mice containing human immunoglobulin genes are immunized with the antigen of interest, e.g. those described herein, lymphatic cells (such as B-cells) from the mice that express antibodies are obtained. Such recovered cells are fused with a myeloid-type cell line to prepare immortal hybridoma cell lines, and such hybridoma cell lines are screened and selected to identify hybridoma cell lines that produce antibodies specific to the antigen of interest. In certain embodiments, the production of a hybridoma cell line that produces antibodies specific to PCSK9 is provided.

In certain embodiments, fully human antibodies can be produced by exposing human splenocytes (B or T cells) to an antigen in vitro, and then reconstituting the exposed cells in an immunocompromised mouse, e.g. SCID or nod/SCID. See, e.g., Brams et al., J. Immunol. 160: 2051-2058 (1998); Carballido et al., Nat. Med., 6: 103-106 (2000). In certain such approaches, engraftment of human fetal tissue into SCID mice (SCID-hu) results in long-term hematopoiesis and human T-cell development. See, e.g., McCune et al., Science, 241:1532-1639 (1988); Ifversen et al., Sem. Immunol., 8:243-248 (1996). In certain instances, humoral immune response in such chimeric mice is dependent on co-development of human T-cells in the animals. See, e.g., Martensson et al., Immunol., 83:1271-179 (1994). In certain approaches, human peripheral blood lymphocytes are transplanted into SCID mice. See, e.g., Mosier et al., Nature, 335:256-259 (1988). In certain such embodiments, when such transplanted cells are treated either with a priming agent, such as Staphylococcal Enterotoxin A (SEA), or with anti-human CD40 monoclonal antibodies, higher levels of B cell production is detected. See, e.g., Martensson et al., Immunol., 84: 224-230 (1995); Murphy et al., Blood, 86:1946-1953 (1995).

Thus, in certain embodiments, fully human antibodies can be produced by the expression of recombinant DNA in host cells or by expression in hybridoma cells. In other embodiments, antibodies can be produced using the phage display techniques described herein.

The antibodies described herein were prepared through the utilization of the XENOMOUSE® technology, as described herein. Such mice, then, are capable of producing human immunoglobulin molecules and antibodies and are deficient in the production of murine immunoglobulin molecules and antibodies. Technologies utilized for achieving the same are disclosed in the patents, applications, and references disclosed in the background section herein. In particular, however, a preferred embodiment of transgenic production of mice and antibodies therefrom is disclosed in U.S. patent application Ser. No. 08/759,620, filed Dec. 3, 1996 and International Patent Application Nos. WO 98/24893, published Jun. 11, 1998 and WO 00/76310, published Dec. 21, 2000, the disclosures of which are hereby incorporated by reference. See also Mendez et al., Nature Genetics, 15:146-156 (1997), the disclosure of which is hereby incorporated by reference.

Through the use of such technology, fully human monoclonal antibodies to a variety of antigens have been produced. Essentially, XENOMOUSE® lines of mice are immunized with an antigen of interest (e.g. an antigen provided herein), lymphatic cells (such as B-cells) are recovered from the hyper-immunized mice, and the recovered lymphocytes are fused with a myeloid-type cell line to prepare immortal hybridoma cell lines. These hybridoma cell lines are screened and selected to identify hybridoma cell lines that produced antibodies specific to the antigen of interest. Provided herein are methods for the production of multiple hybridoma cell lines that produce antibodies specific to PCSK9. Further, provided herein are characterization of the antibodies produced by such cell lines, including nucleotide and amino acid sequence analyses of the heavy and light chains of such antibodies.

The production of the XENOMOUSE® strains of mice is further discussed and delineated in U.S. patent application Ser. No. 07/466,008, filed Jan. 12, 1990, Ser. No. 07/610,515, filed Nov. 8, 1990, Ser. No. 07/919,297, filed Jul. 24, 1992, Ser. No. 07/922,649, filed Jul. 30, 1992, Ser. No. 08/031,801, filed Mar. 15, 1993, Ser. No. 08/112,848, filed Aug. 27, 1993, Ser. No. 08/234,145, filed Apr. 28, 1994, Ser. No. 08/376,279, filed Jan. 20, 1995, Ser. No. 08/430,938, filed Apr. 27, 1995, Ser. No. 08/464,584, filed Jun. 5, 1995, Ser. No. 08/464,582, filed Jun. 5, 1995, Ser. No. 08/463,191, filed Jun. 5, 1995, Ser. No. 08/462,837, filed Jun. 5, 1995, Ser. No. 08/486,853, filed Jun. 5, 1995, Ser. No. 08/486,857, filed Jun. 5, 1995, Ser. No. 08/486,859, filed Jun. 5, 1995, Ser. No. 08/462,513, filed Jun. 5, 1995, Ser. No. 08/724,752, filed Oct. 2, 1996, Ser. No. 08/759,620, filed Dec. 3, 1996, U.S. Publication 2003/0093820, filed Nov. 30, 2001 and U.S. Pat. Nos. 6,162,963, 6,150,584, 6,114,598, 6,075,181, and 5,939,598 and Japanese Patent Nos. 3 068 180 B2, 3 068 506 B2, and 3 068 507 B2. See also European Patent No., EP 0 463 151 B1, grant published Jun. 12, 1996, International Patent Application No., WO 94/02602, published Feb. 3, 1994, International Patent Application No., WO 96/34096, published Oct. 31, 1996, WO 98/24893, published Jun. 11, 1998, WO 00/76310, published Dec. 21, 2000. The disclosures of each of the above-cited patents, applications, and references are hereby incorporated by reference in their entirety.

Using hybridoma technology, antigen-specific human mAbs with the desired specificity can be produced and selected from the transgenic mice such as those described herein. Such antibodies can be cloned and expressed using a suitable vector and host cell, or the antibodies can be harvested from cultured hybridoma cells.

Fully human antibodies can also be derived from phage-display libraries (as described in Hoogenboom et al., (1991) J. Mol. Biol. 227:381; and Marks et al., (1991) J. Mol. Biol. 222:581). Phage display techniques mimic immune selection through the display of antibody repertoires on the surface of filamentous bacteriophage, and subsequent selection of phage by their binding to an antigen of choice. One such technique is described in PCT Publication No. WO 99/10494 (hereby incorporated by reference), which describes the isolation of high affinity and functional agonistic antibodies for MPL- and msk-receptors using such an approach.

### Bispecific or Bifunctional Antigen Binding Proteins

Also provided are bispecific and bifunctional antibodies that include one or more CDRs or one or more variable regions as described above. A bispecific or bifunctional antibody in some instances can be an artificial hybrid antibody having two different heavy/light chain pairs and two different binding sites. Bispecific antibodies can be produced by a variety of methods including, but not limited to, fusion of hybridomas or linking of Fab′ fragments. See, e.g., Songsivilai & Lachmann, (1990) Clin. Exp. Immunol. 79:315-321; Kostelny et al., (1992) J. Immunol. 148:1547-1553. When an antigen binding protein of the instant disclosure binds to PCSK9, the binding may lead to the inhibition of PCSK9 binding to LDLR as described in Example 3.

### Various Other Forms

Some of the antigen binding proteins that specifically bind to PCSK9 that are provided in the present disclosure include variant forms of the antigen binding proteins disclosed herein (e.g., those having the sequences listed in Tables 1-4).

In various embodiments, the antigen binding proteins disclosed herein can comprise one or more non-naturally occurring/encoded amino acids. For instance, some of the antigen binding proteins have one or more non-naturally occurring/encoded amino acid substitutions in one or more of the heavy or light chains, variable regions or CDRs listed in Tables 1-6. Examples of non-naturally occurring/encoded amino acids (which can be substituted for any naturally-occurring amino acid found in any sequence disclosed herein, as desired) include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and imino acids (e.g., 4-hydroxyproline). In the polypeptide notation used herein, the left-hand direction is the amino terminal direction and the right-hand direction is the carboxyl-terminal direction, in accordance with standard usage and convention. A non-limiting lists of examples of non-naturally occurring/encoded amino acids that can be inserted into an antigen binding protein sequence or substituted for a wild-type residue in an antigen binding sequence include β-amino acids, homoamino acids, cyclic amino acids and amino acids with derivatized side chains. Examples include (in the L-form or D-form; abbreviated as in parentheses): citrulline (Cit), homocitrulline (hCit), Nα-methylcitrulline (NMeCit), Nα-methylhomocitrulline (Nα-MeHoCit), ornithine (Orn), Nα-Methylornithine (Nα-MeOrn or NMeOrn), sarcosine (Sar), homolysine (hLys or hK), homoarginine (hArg or hR), homoglutamine (hQ), Nα-methylarginine (NMeR), Nα-methylleucine (Nα-MeL or NMeL), N-methylhomolysine (NMeHoK), Nα-methylglutamine (NMeQ), norleucine (Nle), norvaline (Nva), 1,2,3,4-tetrahydroisoquinoline (Tic), Octahydroindole-2-carboxylic acid (Oic), 3-(1-naphthyl)alanine (1-Nal), 3-(2-naphthyl)alanine (2-Nal), 1,2,3,4-tetrahydroisoquinoline (Tic), 2-indanylglycine (IgI), para-iodophenylalanine (pI-Phe), para-aminophenylalanine (4AmP or 4-Amino-Phe), 4-guanidino phenylalanine (Guf), glycyllysine (abbreviated “K(Nε-glycyl)” or “K(glycyl)” or “K(gly)”), nitrophenylalanine (nitrophe), aminophenylalanine (aminophe or Amino-Phe), benzylphenylalanine (benzylphe), γ-carboxyglutamic acid (γ-carboxyglu), hydroxyproline (hydroxypro), p-carboxyl-phenylalanine (Cpa), α-aminoadipic acid (Aad), Nα-methyl valine (NMeVal), N-α-methyl leucine (NMeLeu), Nα-methylnorleucine (NMeNle), cyclopentylglycine (Cpg), cyclohexylglycine (Chg), acetylarginine (acetylarg), α,β-diaminopropionoic acid (Dpr), α,γ-diaminobutyric acid (Dab), diaminopropionic acid (Dap), cyclohexylalanine (Cha), 4-methyl-phenylalanine (MePhe), β,β-diphenyl-alanine (BiPhA), aminobutyric acid (Abu), 4-phenyl-phenylalanine (or biphenylalanine; 4Bip), α-amino-isobutyric acid (Aib), beta-alanine, beta-aminopropionic acid, piperidinic acid, aminocaprioic acid, aminoheptanoic acid, aminopimelic acid, desmosine, diaminopimelic acid, N-ethylglycine, N-ethylaspargine, hydroxylysine, allo-hydroxylysine, isodesmosine, allo-isoleucine, N-methylglycine, N-methylisoleucine, N-methylvaline, 4-hydroxyproline (Hyp), γ-carboxyglutamate, ε-N,N,N-trimethyllysine, ε-N-acetyllysine, O-phosphoserine, N-acetylserine, N-formylmethionine, 3-methylhistidine, 5-hydroxylysine, ω-methylarginine, 4-Amino-O-Phthalic Acid (4APA), and other similar amino acids, and derivatized forms of any of those specifically listed.

Additionally, the antigen binding proteins can have one or more conservative amino acid substitutions in one or more of the heavy or light chains, variable regions or CDRs listed in Tables 1-6. Naturally-occurring amino acids can be divided into classes based on common side chain properties:

1) hydrophobic: norleucine, Met, Ala, Val, Leu, Ile;

2) neutral hydrophilic: Cys, Ser, Thr, Asn, Gln;

3) acidic: Asp, Glu;

4) basic: His, Lys, Arg;

5) residues that influence chain orientation: Gly, Pro; and

6) aromatic: Trp, Tyr, Phe.

Conservative amino acid substitutions can involve exchange of a member of one of these classes with another member of the same class. Conservative amino acid substitutions can encompass non-naturally occurring/encoded amino acid residues, which are typically incorporated by chemical peptide synthesis rather than by synthesis in biological systems. See Table 8, infra. These include peptidomimetics and other reversed or inverted forms of amino acid moieties.

Non-conservative substitutions can involve the exchange of a member of one of the above classes for a member from another class. Such substituted residues can be introduced into regions of the antibody that are homologous with human antibodies, or into the non-homologous regions of the molecule.

In making such changes, according to certain embodiments, the hydropathic index of amino acids can be considered. The hydropathic profile of a protein is calculated by assigning each amino acid a numerical value (“hydropathy index”) and then repetitively averaging these values along the peptide chain. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic profile in conferring interactive biological function on a protein is understood in the art (see, e.g., Kyte et al., 1982, J. Mol. Biol. 157:105-131). It is known that certain amino acids can be substituted for other amino acids having a similar hydropathic index or score and still retain a similar biological activity. In making changes based upon the hydropathic index, in certain embodiments, the substitution of amino acids whose hydropathic indices are within ±2 is included. In some aspects, those which are within ±1 are included, and in other aspects, those within ±0.5 are included.

It is also understood in the art that the substitution of like amino acids can be made effectively on the basis of hydrophilicity, particularly where the biologically functional protein or peptide thereby created is intended for use in immunological embodiments, as in the present case. In certain embodiments, the greatest local average hydrophilicity of a protein, as governed by the hydrophilicity of its adjacent amino acids, correlates with its immunogenicity and antigen-binding or immunogenicity, that is, with a biological property of the protein.

The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in certain embodiments, the substitution of amino acids whose hydrophilicity values are within ±2 is included, in other embodiments, those which are within ±1 are included, and in still other embodiments, those within ±0.5 are included. In some instances, one can also identify epitopes from primary amino acid sequences on the basis of hydrophilicity. These regions are also referred to as “epitopic core regions.”

Exemplary conservative amino acid substitutions are set forth in Table 8.

TABLE 8
Conservative Amino Acid Substitutions

| Original Residue | Exemplary Substitutions |
|---|---|
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile |
| Phe | Met, Leu, Tyr |
| Ser | Thr |
| Thr | Ser |
| Trp | Tyr |
| Tyr | Trp, Phe |
| Val | Ile, Leu |

A skilled artisan will be able to determine suitable variants of polypeptides as set forth herein using well-known techniques coupled with the information provided herein. One skilled in the art can identify suitable areas of the molecule that can be changed without destroying activity by targeting regions not believed to be important for activity. The skilled artisan also will be able to identify residues and portions of the molecules that are conserved among similar polypeptides. In further embodiments, even areas that can be important for biological activity or for structure can be subject to conservative amino acid substitutions without destroying the biological activity or without adversely affecting the polypeptide structure.

Additionally, one skilled in the art can review structure-function studies identifying residues in similar polypeptides that are important for activity or structure. In view of such a comparison, one can predict the importance of amino acid residues in a protein that correspond to amino acid residues important for activity or structure in similar proteins. One skilled in the art can opt for chemically similar amino acid substitutions for such predicted important amino acid residues.

One skilled in the art can also analyze the three-dimensional structure and amino acid sequence in relation to that structure in similar polypeptides. In view of such information, one skilled in the art can predict the alignment of amino acid residues of an antibody with respect to its three dimensional structure. One skilled in the art can choose not to make radical changes to amino acid residues predicted to be on the surface of the protein, since such residues can be involved in important interactions with other molecules. Moreover, one skilled in the art can generate test variants containing a single amino acid substitution at each desired amino acid residue. These variants can then be screened using assays for inhibition of PCSK9 binding to LDLR, (including those described in the Examples provided herein) thus yielding information regarding which amino acids can be changed and which must not be changed. In other words, based on information gathered from such routine experiments, one skilled in the art can readily determine the amino acid positions where further substitutions should be avoided either alone or in combination with other mutations.

A number of scientific publications have been devoted to the prediction of secondary structure. See, Moult, (1996) Curr. Op. in Biotech. 7:422-427; Chou et al., (1974) Biochem. 13:222-245; Chou et al., (1974) Biochemistry 113:211-222; Chou et al., (1978) Adv. Enzymol. Relat. Areas Mol. Biol. 47:45-148; Chou et al., (1979) Ann. Rev. Biochem. 47:251-276; and Chou et al., (1979) Biophys. J. 26:367-384. Moreover, computer programs are currently available to assist with predicting secondary structure. One method of predicting secondary structure is based upon homology modeling. For example, two polypeptides or proteins that have a sequence identity of greater than 30%, or similarity greater than 40% can have similar structural topologies. The growth of the protein structural database (PDB) has provided enhanced predictability of secondary structure, including the potential number of folds within a polypeptide's or protein's structure. See, Holm et al., (1999) Nucl. Acid. Res. 27:244-247. It has been suggested (Brenner et al., (1997) Curr. Op. Struct. Biol. 7:369-376) that there are a limited number of folds in a given polypeptide or protein and that once a critical number of structures have been resolved, structural prediction will become dramatically more accurate.

Additional methods of predicting secondary structure include “threading” (Jones, (1997) Curr. Opin. Struct. Biol. 7:377-387; Sippl et al., (1996) Structure 4:15-19), “profile analysis” (Bowie et al., (1991) Science 253:164-170; Gribskov et al., (1990) Meth. Enzym. 183:146-159; Gribskov et al., (1987) Proc. Nat. Acad. Sci. 84:4355-4358), and “evolutionary linkage” (See, Holm, (1999) supra; and Brenner, (1997) supra).

In some embodiments, amino acid substitutions are made that: (1) reduce susceptibility to proteolysis, (2) reduce susceptibility to oxidation, (3) alter binding affinity for forming protein complexes, (4) alter ligand or antigen binding affinities, and/or (4) confer or modify other physicochemical or functional properties on such polypeptides. For example, single or multiple amino acid substitutions (in some embodiments, conservative amino acid substitutions) can be made in the naturally-occurring sequence. Substitutions can be made in that portion of the antibody that lies outside the domain(s) forming intermolecular contacts. In such embodiments, conservative amino acid substitutions can be used that do not substantially change the structural characteristics of the parent sequence (e.g., one or more replacement amino acids that do not disrupt the secondary structure that characterizes the parent or native antigen binding protein). Examples of art-recognized polypeptide secondary and tertiary structures are described in Creighton, Proteins: Structures and Molecular Properties 2^{nd} edition, 1992, W. H. Freeman & Company; Creighton, Proteins: Structures and Molecular Principles, 1984, W. H. Freeman & Company; Introduction to Protein Structure (Branden and Tooze, eds.), 2^{nd} edition, 1999, Garland Publishing; Petsko & Ringe, Protein Structure and Function, 2004, New Science Press Ltd; and Thornton et al., (1991) Nature 354:105, which are each incorporated herein by reference.

Additional preferred antibody variants include cysteine variants wherein one or more cysteine residues in the parent or native amino acid sequence are deleted from or substituted with another amino acid (e.g., serine). Cysteine variants are useful, inter alia when antibodies must be refolded into a biologically active conformation. Cysteine variants can have fewer cysteine residues than the native antibody, and typically have an even number to minimize interactions resulting from unpaired cysteines.

The heavy and light chains, variable regions domains and CDRs that are disclosed can be used to prepare polypeptides that contain an antigen binding region that can specifically bind to a PCSK9 and inhibit PCSK9 binding to LDLR. For example, one or more of the CDRs listed in Tables 3-6 can be incorporated into a molecule (e.g., a polypeptide) covalently or noncovalently to make an immunoadhesion. An immunoadhesion can incorporate the CDR(s) as part of a larger polypeptide chain, can covalently link the CDR(s) to another polypeptide chain, or can incorporate the CDR(s) noncovalently. The CDR(s) enable the immunoadhesion to bind specifically to a particular antigen of interest (e.g., to PCSK9, including an epitope thereon).

The heavy and light chains, variable regions domains and CDRs that are disclosed can be used to prepare polypeptides that contain an antigen binding region that can specifically bind to PCSK9 and inhibit PCSK9 from binding to LDLR. For example, one or more of the CDRs listed in Tables 3-6 can be incorporated into a molecule (e.g., a polypeptide) that is structurally similar to a “half” antibody comprising the heavy chain, the light chain of an antigen binding protein paired with a Fc fragment so that the antigen binding region is monovalent (like a Fab fragment) but with a dimeric Fc moiety.

Mimetics (e.g., “peptide mimetics” or “peptidomimetics”) based upon the variable region domains and CDRs that are described herein are also provided. These analogs can be peptides, non-peptides or combinations of peptide and non-peptide regions. Fauchere, (1986) Adv. Drug Res. 15:29; Veber and Freidinger, (1985) TINS p. 392; and Evans et al., (1987) J. Med. Chem. 30:1229, which are incorporated herein by reference for any purpose. Peptide mimetics that are structurally similar to therapeutically useful peptides can be used to produce a similar therapeutic or prophylactic effect. Such compounds are often developed with the aid of computerized molecular modeling. Generally, peptidomimetics are proteins that are structurally similar to an antibody displaying a desired biological activity, such as the ability to specifically bind to PCSK9, but have one or more peptide linkages optionally replaced by a linkage selected from: —CH₂NH—, —CH₂S—, —CH₂—CH₂—, —CH—CH-(cis and trans), —COCH₂—, —CH(OH)CH₂—, and —CH₂SO—, by methods well known in the art. Systematic substitution of one or more amino acids of a consensus sequence with a D-amino acid of the same type (e.g., D-lysine in place of L-lysine) can be used in certain embodiments to generate more stable proteins. In addition, constrained peptides comprising a consensus sequence or a substantially identical consensus sequence variation can be generated by methods known in the art (Rizo and Gierasch, (1992) Ann. Rev. Biochem. 61:387), incorporated herein by reference), for example, by adding internal cysteine residues capable of forming intramolecular disulfide bridges which cyclize the peptide.

Derivatives of the antigen binding proteins that specifically bind to PCSK9 that are described herein are also provided. The derivatized antigen binding proteins can comprise any molecule or substance that imparts a desired property to the antibody or fragment, such as increased half-life in a particular use. The derivatized antigen binding protein can comprise, for example, a detectable (or labeling) moiety (e.g., a radioactive, colorimetric, antigenic or enzymatic molecule, a detectable bead (such as a magnetic or electrodense (e.g., gold) bead), or a molecule that binds to another molecule (e.g., biotin or streptavidin), a therapeutic or diagnostic moiety (e.g., a radioactive, cytotoxic, or pharmaceutically active moiety), or a molecule that increases the suitability of the antigen binding protein for a particular use (e.g., administration to a subject, such as a human subject, or other in vivo or in vitro uses). Examples of molecules that can be used to derivatize an antigen binding protein include albumin (e.g., human serum albumin) and polyethylene glycol (PEG). Albumin-linked and PEGylated derivatives of antigen binding proteins can be prepared using techniques well known in the art. Certain antigen binding proteins include a PEGylated single chain polypeptide as described herein. In one embodiment, the antigen binding protein is conjugated or otherwise linked to transthyretin (“TTR”) or a TTR variant. The TTR or TTR variant can be chemically modified with, for example, a chemical selected from the group consisting of dextran, poly(n-vinyl pyrrolidone), polyethylene glycols, propropylene glycol homopolymers, polypropylene oxide/ethylene oxide co-polymers, polyoxyethylated polyols and polyvinyl alcohols.

Other derivatives include covalent or aggregative conjugates of the antigen binding proteins that specifically bind to PCSK9 that are disclosed herein with other proteins or polypeptides, such as by expression of recombinant fusion proteins comprising heterologous polypeptides fused to the N-terminus or C-terminus of an antigen binding protein that inhibits PCSK9 from binding LDLR. For example, the conjugated peptide can be a heterologous signal (or leader) polypeptide, e.g., the yeast alpha-factor leader, or a peptide such as an epitope tag. An antigen binding protein-containing fusion protein of the present disclosure can comprise peptides added to facilitate purification or identification of an antigen binding protein that specifically binds to PCSK9 (e.g., a poly-His tag) and that inhibits PSCK9 binding to LDLR. An antigen binding protein that specifically binds to PCSK9 also can be linked to the FLAG peptide as described in Hopp et al., (1988) Bio/Technology 6:1204; and U.S. Pat. No. 5,011,912. The FLAG peptide is highly antigenic and provides an epitope reversibly bound by a specific monoclonal antibody (mAb), enabling rapid assay and facile purification of expressed recombinant protein. Reagents useful for preparing fusion proteins in which the FLAG peptide is fused to a given polypeptide are commercially available (Sigma, St. Louis, Mo.).

Multimers that comprise one or more antigen binding proteins that specifically bind to PCSK9 form another aspect of the present disclosure. Multimers can take the form of covalently-linked or non-covalently-linked dimers, trimers, or higher multimers. Multimers comprising two or more antigen binding proteins that bind to PCSK9 and which inhibit PCSK9 binding to LDLR are contemplated for use as therapeutics, diagnostics and for other uses as well, with one example of such a multimer being a homodimer. Other exemplary multimers include heterodimers, homotrimers, heterotrimers, homotetramers, heterotetramers, etc.

One embodiment is directed to multimers comprising multiple antigen binding proteins that specifically bind to PCSK9 joined via covalent or non-covalent interactions between peptide moieties fused to an antigen binding protein that specifically binds to PCSK9. Such peptides can be peptide linkers (spacers), or peptides that have the property of promoting multimerization. Leucine zippers and certain polypeptides derived from antibodies are among the peptides that can promote multimerization of antigen binding proteins attached thereto, as described in more detail herein.

In particular embodiments, the multimers comprise from two to four antigen binding proteins that bind to PCSK9. The antigen binding protein moieties of the multimer can be in any of the forms described above, e.g., variants or fragments. Preferably, the multimers comprise antigen binding proteins that have the ability to specifically bind to PCSK9.

In one embodiment, an oligomer is prepared using polypeptides derived from immunoglobulins. Preparation of fusion proteins comprising certain heterologous polypeptides fused to various portions of antibody-derived polypeptides (including the Fc domain) has been described, e.g., by Ashkenazi et al., (1991) Proc. Natl. Acad. Sci. USA 88:10535; Byrn et al., (1990) Nature 344:677; and Hollenbaugh et al., (1992) Current Protocols in Immunology, Suppl. 4, pages 10.19.1-10.19.11.

One embodiment comprises a dimer comprising two fusion proteins created by fusing an antigen binding protein that specifically binds to PCSK9 to the Fc region of an antibody. The dimer can be made by, for example, inserting a gene fusion encoding the fusion protein into an appropriate expression vector, expressing the gene fusion in host cells transformed with the recombinant expression vector, and allowing the expressed fusion protein to assemble much like antibody molecules, whereupon interchain disulfide bonds form between the Fc moieties to yield the dimer

The term “Fc polypeptide” as used herein includes native and mutein forms of polypeptides derived from the Fc region of an antibody. Truncated forms of such polypeptides containing the hinge region that promotes dimerization also are included. Fusion proteins comprising Fc moieties (and oligomers formed therefrom) offer the advantage of facile purification by affinity chromatography over Protein A or Protein G columns.

One suitable Fc polypeptide, described in PCT application WO 93/10151 and U.S. Pat. No. 5,426,048 and U.S. Pat. No. 5,262,522, is a single chain polypeptide extending from the N-terminal hinge region to the native C-terminus of the Fc region of a human IgG1 antibody. Another useful Fc polypeptide is the Fc mutein described in U.S. Pat. No. 5,457,035, and in Baum et al., (1994) EMBO J. 13:3992-4001. The amino acid sequence of this mutein is identical to that of the native Fc sequence presented in WO 93/10151, except that amino acid 19 has been changed from Leu to Ala, amino acid 20 has been changed from Leu to Glu, and amino acid 22 has been changed from Gly to Ala. The mutein exhibits reduced affinity for Fc receptors.

In other embodiments, the variable portion of the heavy and/or light chains of a antigen binding protein such as disclosed herein can be substituted for the variable portion of an antibody heavy and/or light chain.

Alternatively, the oligomer is a fusion protein comprising multiple antigen binding proteins that specifically bind to PCSK9 with or without peptide linkers (spacer peptides). Among the suitable peptide linkers are those described in U.S. Pat. No. 4,751,180 and U.S. Pat. No. 4,935,233.

Another method for preparing oligomeric derivatives comprising that antigen binding proteins that specifically bind to a PCSK9 involves use of a leucine zipper. Leucine zipper domains are peptides that promote oligomerization of the proteins in which they are found. Leucine zippers were originally identified in several DNA-binding proteins (Landschultz et al., (1988) Science 240:1759-64), and have since been found in a variety of different proteins. Among the known leucine zippers are naturally occurring peptides and derivatives thereof that dimerize or trimerize. Examples of leucine zipper domains suitable for producing soluble oligomeric proteins are described in PCT application WO 94/10308, and the leucine zipper derived from lung surfactant protein D (SPD) described in Hoppe et al., (1994) FEBS Letters 344:191, hereby incorporated by reference. The use of a modified leucine zipper that allows for stable trimerization of a heterologous protein fused thereto is described in Fanslow et al., (1994) Semin. Immunol. 6:267-278. In one approach, recombinant fusion proteins comprising an antigen binding protein fragment or derivative that specifically binds to PCSK9 is fused to a leucine zipper peptide are expressed in suitable host cells, and the soluble oligomeric antigen binding protein fragments or derivatives that form are recovered from the culture supernatant.

In certain embodiments, the antigen binding protein has a K_{D} (equilibrium binding affinity) of less than 1 pM, 10 pM, 100 pM, 1 nM, 2 nM, 5 nM, 10 nM, 25 nM or 50 nM.

In another aspect the instant disclosure provides an antigen binding protein having a half-life of at least one day in vitro or in vivo (e.g., when administered to a human subject). In one embodiment, the antigen binding protein has a half-life of at least three days. In another embodiment, the antibody or portion thereof has a half-life of four days or longer. In another embodiment, the antibody or portion thereof has a half-life of eight days or longer. In another embodiment, the antibody or portion thereof has a half-life of ten days or longer. In another embodiment, the antibody or portion thereof has a half-life of eleven days or longer. In another embodiment, the antibody or portion thereof has a half-life of fifteen days or longer. In another embodiment, the antibody or antigen-binding portion thereof is derivatized or modified such that it has a longer half-life as compared to the underivatized or unmodified antibody. In another embodiment, an antigen binding protein that specifically binds PCSK9 contains point mutations to increase serum half life, such as described in WO 00/09560, published Feb. 24, 2000, incorporated by reference.

### Glycosylation

An antigen binding protein that specifically binds to PCSK9 can have a glycosylation pattern that is different or altered from that found in the native species. As is known in the art, glycosylation patterns can depend on both the sequence of the protein (e.g., the presence or absence of particular glycosylation amino acid residues, discussed below), or the host cell or organism in which the protein is produced. Particular expression systems are discussed below.

Glycosylation of polypeptides is typically either N-linked or O-linked. N-linked refers to the attachment of the carbohydrate moiety to the side chain of an asparagine residue. The tri-peptide sequences asparagine-X-serine and asparagine-X-threonine, where X is any amino acid except proline, are the recognition sequences for enzymatic attachment of the carbohydrate moiety to the asparagine side chain. Thus, the presence of either of these tri-peptide sequences in a polypeptide creates a potential glycosylation site. O-linked glycosylation refers to the attachment of one of the sugars N-acetylgalactosamine, galactose, or xylose, to a hydroxyamino acid, most commonly serine or threonine, although 5-hydroxyproline or 5-hydroxylysine can also be used.

Addition of glycosylation sites to the antigen binding protein is conveniently accomplished by altering the amino acid sequence such that it contains one or more of the above-described tri-peptide sequences (for N-linked glycosylation sites). The alteration can also be made by the addition of, or substitution by, one or more serine or threonine residues to the starting sequence (for O-linked glycosylation sites). For ease, the antigen binding protein amino acid sequence can be altered through changes at the DNA level, particularly by mutating the DNA encoding the target polypeptide at preselected bases such that codons are generated that will translate into the desired amino acids.

Another means of increasing the number of carbohydrate moieties on the antigen binding protein is by chemical or enzymatic coupling of glycosides to the protein. These procedures are advantageous in that they do not require production of the protein in a host cell that has glycosylation capabilities for N- and O-linked glycosylation. Depending on the coupling mode used, the sugar(s) can be attached to (a) arginine and histidine; (b) free carboxyl groups; (c) free sulfhydryl groups such as those of cysteine; (d) free hydroxyl groups such as those of serine, threonine, or hydroxyproline; (e) aromatic residues such as those of phenylalanine, tyrosine, or tryptophan; or (f) the amide group of glutamine. These methods are described in WO 87/05330 and in Aplin & Wriston, (1981) CRC Crit. Rev. Biochem. 10:259-306.

Removal of carbohydrate moieties present on the starting antigen binding protein can be accomplished chemically or enzymatically. Chemical deglycosylation requires exposure of the protein to the compound trifluoromethanesulfonic acid, or an equivalent compound. This treatment results in the cleavage of most or all sugars except the linking sugar (N-acetylglucosamine or N-acetylgalactosamine), while leaving the polypeptide intact. Chemical deglycosylation is described by Hakimuddin et al., (1987) Arch. Biochem. Biophys. 259:52-57 and by Edge et al., (1981) Anal. Biochem. 118:131-37. Enzymatic cleavage of carbohydrate moieties on polypeptides can be achieved by the use of a variety of endo- and exo-glycosidases as described by Thotakura et al., (1987) Meth. Enzymol. 138:350-59. Glycosylation at potential glycosylation sites can be prevented by the use of the compound tunicamycin as described by Duskin et al., (1982) J. Biol. Chem. 257:3105-09. Tunicamycin blocks the formation of protein-N-glycoside linkages.

Hence, aspects of the present disclosure include glycosylation variants of antigen binding proteins that specifically bind to PCSK9 wherein the number and/or type of glycosylation site(s) has been altered compared to the amino acid sequences of the parent polypeptide. In certain embodiments, antibody protein variants comprise a greater or a lesser number of N-linked glycosylation sites than the native antibody. An N-linked glycosylation site is characterized by the sequence: Asn-X-Ser or Asn-X-Thr, wherein the amino acid residue designated as X can be any amino acid residue except proline. The substitution of amino acid residues to create this sequence provides a potential new site for the addition of an N-linked carbohydrate chain. Alternatively, substitutions that eliminate or alter this sequence will prevent addition of an N-linked carbohydrate chain present in the native polypeptide. For example, the glycosylation can be reduced by the deletion of an Asn or by substituting the Asn with a different amino acid. In other embodiments, one or more new N-linked sites are created. Antibodies typically have a N-linked glycosylation site in the Fc region.

### Labels and Effector Groups

In some embodiments, an antigen binding protein that specifically binds to PCSK9 comprises one or more labels. The term “labeling group” or “label” means any detectable label. Examples of suitable labeling groups include, but are not limited to, the following: radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I) fluorescent groups (e.g., FITC, rhodamine, lanthanide phosphors), enzymatic groups (e.g., horseradish peroxidase, β-galactosidase, luciferase, alkaline phosphatase), chemiluminescent groups, biotinyl groups, or predetermined polypeptide epitopes recognized by a secondary reporter (e.g., leucine zipper pair sequences, binding sites for secondary antibodies, metal binding domains, epitope tags). In some embodiments, the labeling group is coupled to the antigen binding protein via spacer arms of various lengths to reduce potential steric hindrance. Various methods for labeling proteins are known in the art and can be used as is seen fit.

The term “effector group” means any group coupled to an antigen binding protein that specifically binds PCSK9 and that acts as a cytotoxic agent. Examples for suitable effector groups are radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I). Other suitable groups include toxins, therapeutic groups, or chemotherapeutic groups. Examples of suitable groups include calicheamicin, auristatins, geldanamycin and cantansine. In some embodiments, the effector group is coupled to the antigen binding protein via spacer arms of various lengths to reduce potential steric hindrance.

In general, labels fall into a variety of classes, depending on the assay in which they are to be detected: a) isotopic labels, which can be radioactive or heavy isotopes; b) magnetic labels (e.g., magnetic particles); c) redox active moieties; d) optical dyes; enzymatic groups (e.g. horseradish peroxidase, β-galactosidase, luciferase, alkaline phosphatase); e) biotinylated groups; and f) predetermined polypeptide epitopes recognized by a secondary reporter (e.g., leucine zipper pair sequences, binding sites for secondary antibodies, metal binding domains, epitope tags, etc.). In some embodiments, the labeling group is coupled to the antigen binding protein via spacer arms of various lengths to reduce potential steric hindrance. Various methods for labeling proteins are known in the art.

Specific labels include optical dyes, including, but not limited to, chromophores, phosphors and fluorophores, with the latter being specific in many instances. Fluorophores can be either “small molecule” fluores, or proteinaceous fluores.

By “fluorescent label” is meant any molecule that can be detected via its inherent fluorescent properties. Suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue, Texas Red, IAEDANS, EDANS, BODIPY FL, LC Red 640, Cy 5, Cy 5.5, LC Red 705, Oregon green, the Alexa-Fluor dyes (Alexa Fluor 350, Alexa Fluor 430, Alexa Fluor 488, Alexa Fluor 546, Alexa Fluor 568, Alexa Fluor 594, Alexa Fluor 633, Alexa Fluor 647, Alexa Fluor 660, Alexa Fluor 680), Cascade Blue, Cascade Yellow and R-phycoerythrin (PE) (Molecular Probes, Eugene, Oreg.), FITC, Rhodamine, and Texas Red (Pierce, Rockford, Ill.), Cy5, Cy5.5, Cy7 (Amersham Life Science, Pittsburgh, Pa.). Suitable optical dyes, including fluorophores, are described in Molecular Probes Handbook by Richard P. Haugland and in subsequent editions, including Molecular Probes Handbook, A Guide to Fluorescent Probes and Labeling Technologies, 11^{th} edition, Johnson and Spence (eds), hereby expressly incorporated by reference.

Suitable proteinaceous fluorescent labels also include, but are not limited to, green fluorescent protein, including a Renilla, Ptilosarcus, or Aequorea species of GFP (Chalfie et al., (1994) Science 263:802-805), eGFP (Clontech Labs., Inc., Genbank Accession Number U55762), blue fluorescent protein (BFP, Quantum Biotechnologies, Inc., Quebec, Canada; Stauber, (1998) Biotechniques 24:462-71; Heim et al., (1996) Curr. Biol. 6:178-82), enhanced yellow fluorescent protein (EYFP, Clontech Labs., Inc.), luciferase (Ichiki et al., (1993) J. Immunol. 150:5408-17), β-galactosidase (Nolan et al., (1988) Proc. Natl. Acad. Sci. U.S.A. 85:2603-07) and Renilla (WO92/15673, WO95/07463, WO98/14605, WO98/26277, WO99/49019, U.S. Pat. Nos. 5,292,658, 5,418,155, 5,683,888, 5,741,668, 5,777,079, 5,804,387, 5,874,304, 5,876,995 and 5,925,558).

### Preparing of Antigen Binding Proteins

Non-human antibodies that are provided can be, for example, derived from any antibody-producing animal, such as a mouse, rat, rabbit, goat, donkey, or non-human primate (such as a monkey, (e.g., cynomolgus or rhesus monkey) or an ape (e.g., chimpanzee)). Non-human antibodies can be used, for instance, in in vitro cell culture and cell-culture based applications, or any other application where an immune response to the antibody does not occur or is insignificant, can be prevented, is not a concern, or is desired. In certain embodiments, the antibodies can be produced by immunizing with recombinant self-cleaved, mature, secreted PCSK9 comprising amino acids 31 to 692 of the amino acid sequence of SEQ ID NO: 2; or with full-length PCSK9; or with whole cells expressing PCSK9; or with membranes prepared from cells expressing PCSK9; or with fusion proteins, e.g., Fc fusions comprising PCSK9 (or extracellular domains thereof) fused to Fc, and other methods known in the art, e.g., as described in the Examples presented herein. Alternatively, the certain non-human antibodies can be raised by immunizing with amino acids which are segments PCSK9 that form part of the epitope to which certain antibodies provided herein bind. The antibodies can be polyclonal, monoclonal, or can be synthesized in host cells by expressing recombinant DNA.

Fully human antibodies can be prepared as described above by immunizing transgenic animals containing human immunoglobulin loci or by selecting a phage display library that is expressing a repertoire of human antibodies.

The monoclonal antibodies (mAbs) can be produced by a variety of techniques, including conventional monoclonal antibody methodology, e.g., the standard somatic cell hybridization technique of Kohler & Milstein, (1975) Nature 256:495-97. Alternatively, other techniques for producing monoclonal antibodies can be employed, for example, the viral or oncogenic transformation of B-lymphocytes. One suitable animal system for preparing hybridomas is the murine system, which is a very well established procedure. Immunization protocols and techniques for isolation of immunized splenocytes for fusion are known in the art. For such procedures, B cells from immunized mice are fused with a suitable immortalized fusion partner, such as a murine myeloma cell line. If desired, rats or other mammals besides can be immunized instead of mice and B cells from such animals can be fused with the murine myeloma cell line to form hybridomas. Alternatively, a myeloma cell line from a source other than mouse can be used. Fusion procedures for making hybridomas also are well known. SLAM technology can also be employed in the production of antibodies.

The single chain antibodies that are provided can be formed by linking heavy and light chain variable domain (Fv region) fragments via an amino acid bridge (short peptide linker), resulting in a single polypeptide chain. Such single-chain Fvs (scFvs) can be prepared by fusing DNA encoding a peptide linker between DNAs encoding the two variable domain polypeptides (V_{L} and V_{H}). The resulting polypeptides can fold back on themselves to form antigen-binding monomers, or they can form multimers (e.g., dimers, trimers, or tetramers), depending on the length of a flexible linker between the two variable domains (Kortt et al., (1997) Prot. Eng. 10:423; Kortt et al., (2001) Biomol. Eng. 18:95-108). By combining different V_{L} and V_{H}-comprising polypeptides, one can form multimeric scFvs that bind to different epitopes (Kriangkum et al., (2001) Biomol. Eng. 18:31-40). Techniques developed for the production of single chain antibodies include those described in U.S. Pat. No. 4,946,778; Bird et al., (1988) Science 242:423-26; Huston et al., (1988) Proc. Natl. Acad. Sci. U.S.A. 85:5879-83; Ward et al., (1989) Nature 334:544-46, de Graaf et al., (2002) Methods Mol Biol. 178:379-387. Single chain antibodies derived from antibodies provided herein include, but are not limited to scFvs comprising the variable domain combinations of the heavy and light chain variable regions depicted in Table 2, or combinations of light and heavy chain variable domains which include the CDRs depicted in Tables 3-4 and 6-23.

Antibodies provided herein that are of one subclass can be changed to antibodies from a different subclass using subclass switching methods. Thus, IgG antibodies can be derived from an IgM antibody, for example, and vice versa. Such techniques allow the preparation of new antibodies that possess the antigen binding properties of a given antibody (the parent antibody), but also exhibit biological properties associated with an antibody isotype or subclass different from that of the parent antibody. Recombinant DNA techniques can be employed. Cloned DNA encoding particular antibody polypeptides can be employed in such procedures, e.g., DNA encoding the constant domain of an antibody of the desired isotype. See, e.g., Lantto et al., (2002) Methods Mol. Biol. 178:303-16.

Accordingly, the antibodies that are provided include those comprising, for example, the variable domain combinations described, supra., having a desired isotype (for example, IgA, IgG1, IgG2, IgG3, IgG4, IgE, and IgD) as well as Fab or F(ab′)₂ fragments thereof. Moreover, if an IgG4 is desired, it can also be desired to introduce a point mutation (e.g., a mutation from CPSCP to CPPCP (SEQ ID NOs 1828 and 1829, respectively, in order of appearance) in the hinge region as described in Bloom et al., (1997) Protein Science 6:407-15, incorporated by reference herein) to alleviate a tendency to form intra-H chain disulfide bonds that can lead to heterogeneity in the IgG4 antibodies.

Moreover, techniques for deriving antibodies having different properties (i.e., varying affinities for the antigen to which they bind) are also known. One such technique, referred to as chain shuffling, involves displaying immunoglobulin variable domain gene repertoires on the surface of filamentous bacteriophage, often referred to as phage display. Chain shuffling has been used to prepare high affinity antibodies to the hapten 2-phenyloxazol-5-one, as described by Marks et al., (1992) Nature Biotechnology 10:779-83.

Conservative modifications can be made to the heavy and light chain variable regions described in Table 2, or the CDRs described in Tables 3A and 3B, and 4A and 4B.6-23 (and corresponding modifications to the encoding nucleic acids) to produce an antigen binding protein having functional and biochemical characteristics. Methods for achieving such modifications are described herein.

Antigen binding proteins that specifically bind to PCSK9 can be further modified in various ways. For example, if they are to be used for therapeutic purposes, they can be conjugated with polyethylene glycol (PEGylated) to prolong the serum half-life or to enhance protein delivery. PEG can be attached directly to the antigen binding protein or it can be attached via a linker, such as a glycosidic linkage.

Alternatively, the V region of the subject antibodies or fragments thereof can be fused with the Fc region of a different antibody molecule. The Fc region used for this purpose can be modified so that it does not bind complement, thus reducing the likelihood of inducing cell lysis in the patient when the fusion protein is used as a therapeutic agent. In addition, the subject antibodies or functional fragments thereof can be conjugated with human serum albumin to enhance the serum half-life of the antibody or fragment thereof. Another useful fusion partner for the antigen binding proteins or fragments thereof is transthyretin (TTR). TTR has the capacity to form a tetramer, thus an antibody-TTR fusion protein can form a multivalent antibody which can increase its binding avidity.

Alternatively, substantial modifications in the functional and/or biochemical characteristics of the antigen binding proteins described herein can be achieved by creating substitutions in the amino acid sequence of the heavy and light chains that differ significantly in their effect on maintaining (a) the structure of the molecular backbone in the area of the substitution, for example, as a sheet or helical conformation, (b) the charge or hydrophobicity of the molecule at the target site, or (c) the bulkiness of the side chain. A “conservative amino acid substitution” can involve a substitution of a native amino acid residue with a nonnative residue that has little or no effect on the polarity or charge of the amino acid residue at that position. See, Table 8, supra. Furthermore, any native residue in the polypeptide can also be substituted with alanine, as has been previously described for alanine scanning mutagenesis.

Amino acid substitutions (whether conservative or non-conservative) of the subject antibodies can be implemented by those skilled in the art by applying routine techniques. Amino acid substitutions can be used to identify important residues of the antibodies provided herein, or to increase or decrease the affinity of these antibodies PCSK9 or for modifying the binding affinity of other antigen-binding proteins described herein.

### Methods of Expressing Antigen Binding Proteins

Expression systems and constructs in the form of plasmids, expression vectors, transcription or expression cassettes that comprise at least one polynucleotide as described above are also provided herein, as well host cells comprising such expression systems or constructs.

The antigen binding proteins provided herein can be prepared by any of a number of conventional techniques. For example, antigen binding proteins that specifically bind to PCSK9 can be produced by recombinant expression systems, using any technique known in the art. See, e.g., Monoclonal Antibodies, Hybridomas: A New Dimension in Biological Analyses, (Kennet et al., eds.) Plenum Press (1980) and subsequent editions; and Harlow & Lane, (1988) supra.

Antigen binding proteins can be expressed in hybridoma cell lines (e.g., in particular antibodies can be expressed in hybridomas) or in cell lines other than hybridomas. Expression constructs encoding the antibodies can be used to transform a mammalian, insect or microbial host cell. Transformation can be performed using any known method for introducing polynucleotides into a host cell, including, for example packaging the polynucleotide in a virus or bacteriophage and transducing a host cell with the construct by transfection procedures known in the art, as exemplified by U.S. Pat. Nos. 4,399,216; 4,912,040; 4,740,461; and 4,959,455. The optimal transformation procedure used will depend upon which type of host cell is being transformed. Methods for introduction of heterologous polynucleotides into mammalian cells are well known in the art and include, but are not limited to, dextran-mediated transfection, calcium phosphate precipitation, polybrene mediated transfection, protoplast fusion, electroporation, encapsulation of the polynucleotide(s) in liposomes, mixing nucleic acid with positively-charged lipids, and direct microinjection of the DNA into nuclei.

Recombinant expression constructs typically comprise a nucleic acid molecule encoding a polypeptide comprising one or more of the following: one or more CDRs provided herein; a light chain constant region; a light chain variable region; a heavy chain constant region (e.g., C_{H}1, C_{H}2 and/or C_{H}3); and/or another scaffold portion of an antigen binding protein. These nucleic acid sequences are inserted into an appropriate expression vector using standard ligation techniques. In one embodiment, the heavy or light chain constant region is appended to the C-terminus of the anti-PCSK9 specific heavy or light chain variable region and is ligated into an expression vector. The vector is typically selected to be functional in the particular host cell employed (i.e., the vector is compatible with the host cell machinery, permitting amplification and/or expression of the gene can occur). In some embodiments, vectors are used that employ protein-fragment complementation assays using protein reporters, such as dihydrofolate reductase (see, for example, U.S. Pat. No. 6,270,964, which is hereby incorporated by reference). Suitable expression vectors can be purchased, for example, from Invitrogen Life Technologies or BD Biosciences. Other useful vectors for cloning and expressing the antibodies and fragments include those described in Bianchi and McGrew, (2003) Biotech. Biotechnol. Bioeng. 84:439-44, which is hereby incorporated by reference. Additional suitable expression vectors are discussed, for example, in “Gene Expression Technology,” Methods Enzymol., vol. 185, (Goeddel et al., ed.), (1990), Academic Press.

Typically, expression vectors used in any of the host cells will contain sequences for plasmid maintenance and for cloning and expression of exogenous nucleotide sequences. Such sequences, collectively referred to as “flanking sequences” in certain embodiments will typically include one or more of the following nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element.

Optionally, an expression vector can contain a “tag”-encoding sequence, i.e., an oligonucleotide molecule located at the 5′ or 3′ end of an antigen binding protein coding sequence; the oligonucleotide sequence encodes polyHis (such as hexaHis, HHHHHH (SEQ ID NO: 72)), or another “tag” such as FLAG, HA (hemaglutinin influenza virus), or myc, for which commercially available antibodies exist. This tag is typically fused to the polypeptide upon expression of the polypeptide, and can serve as a means for affinity purification or detection of the antigen binding protein from the host cell. Affinity purification can be accomplished, for example, by column chromatography using antibodies against the tag as an affinity matrix. Optionally, the tag can subsequently be removed from the purified antigen binding protein by various means such as using certain peptidases for cleavage.

Flanking sequences can be homologous (i.e., from the same species and/or strain as the host cell), heterologous (i.e., from a species other than the host cell species or strain), hybrid (i.e., a combination of flanking sequences from more than one source), synthetic or native. As such, the source of a flanking sequence can be any prokaryotic or eukaryotic organism, any vertebrate or invertebrate organism, or any plant, provided that the flanking sequence is functional in, and can be activated by, the host cell machinery.

Flanking sequences useful in the vectors can be obtained by any of several methods well known in the art. Typically, flanking sequences useful herein will have been previously identified by mapping and/or by restriction endonuclease digestion and can thus be isolated from the proper tissue source using the appropriate restriction endonucleases. In some cases, the full nucleotide sequence of a flanking sequence can be known. Here, the flanking sequence can be synthesized using the methods described herein for nucleic acid synthesis or cloning.

Whether all or only a portion of the flanking sequence is known, it can be obtained using polymerase chain reaction (PCR) and/or by screening a genomic library with a suitable probe such as an oligonucleotide and/or flanking sequence fragment from the same or another species. Where the flanking sequence is not known, a fragment of DNA containing a flanking sequence can be isolated from a larger piece of DNA that can contain, for example, a coding sequence or even another gene or genes. Isolation can be accomplished by restriction endonuclease digestion to produce the proper DNA fragment followed by isolation using agarose gel purification, column chromatography or other methods known to the skilled artisan. The selection of suitable enzymes to accomplish this purpose will be readily apparent to one of ordinary skill in the art.

An origin of replication is typically a part of those prokaryotic expression vectors purchased commercially, and the origin aids in the amplification of the vector in a host cell. If the vector of choice does not contain an origin of replication site, one can be chemically synthesized based on a known sequence, and ligated into the vector. For example, the origin of replication from the plasmid pBR322 (GenBank Accession # J01749, New England Biolabs, Beverly, Mass.) is suitable for most gram-negative bacteria, and various viral origins (e.g., SV40, polyoma, adenovirus, vesicular stomatitus virus (VSV), or papilloma viruses such as HPV or BPV) are useful for cloning vectors in mammalian cells. Generally, the origin of replication component is not needed for mammalian expression vectors (for example, the SV40 origin is often used only because it also contains the virus early promoter).

A transcription termination sequence is typically located 3′ to the end of a polypeptide coding region and serves to terminate transcription. Usually, a transcription termination sequence in prokaryotic cells is a G-C rich fragment followed by a poly-T sequence. While the sequence is easily cloned from a library or even purchased commercially as part of a vector, it can also be readily synthesized using methods for nucleic acid synthesis such as those described herein.

A selectable marker gene encodes a protein necessary for the survival and growth of a host cell grown in a selective culture medium. Typical selection marker genes encode proteins that (a) confer resistance to antibiotics or other toxins, e.g., ampicillin, tetracycline, or kanamycin for prokaryotic host cells; (b) complement auxotrophic deficiencies of the cell; or (c) supply critical nutrients not available from complex or defined media. Specific selectable markers are the kanamycin resistance gene, the ampicillin resistance gene, and the tetracycline resistance gene. Advantageously, a neomycin resistance gene can also be used for selection in both prokaryotic and eukaryotic host cells.

Other selectable genes can be used to amplify the gene that will be expressed. Amplification is the process wherein genes that are required for production of a protein critical for growth or cell survival are reiterated in tandem within the chromosomes of successive generations of recombinant cells. Examples of suitable selectable markers for mammalian cells include dihydrofolate reductase (DHFR) and promoterless thymidine kinase genes. Mammalian cell transformants are placed under selection pressure wherein only the transformants are uniquely adapted to survive by virtue of the selectable gene present in the vector. Selection pressure is imposed by culturing the transformed cells under conditions in which the concentration of selection agent in the medium is successively increased, thereby leading to the amplification of both the selectable gene and the DNA that encodes another gene, such as an antigen binding protein that binds to PCSK9. As a result, increased quantities of a polypeptide such as an antigen binding protein are synthesized from the amplified DNA.

A ribosome-binding site is usually necessary for translation initiation of mRNA and is characterized by a Shine-Dalgarno sequence (prokaryotes) or a Kozak sequence (eukaryotes). The element is typically located 3′ to the promoter and 5′ to the coding sequence of the polypeptide to be expressed.

In some cases, such as where glycosylation is desired in a eukaryotic host cell expression system, one can manipulate the various pre- or pro-sequences to improve glycosylation or yield. For example, one can alter the peptidase cleavage site of a particular signal peptide, or add prosequences, which also can affect glycosylation. The final protein product can have, in the −1 position (relative to the first amino acid of the mature protein), one or more additional amino acids incident to expression, which may not have been totally removed. For example, the final protein product can have one or two amino acid residues found in the peptidase cleavage site, attached to the amino-terminus. Alternatively, use of some enzyme cleavage sites can result in a slightly truncated form of the desired polypeptide, if the enzyme cuts at such area within the mature polypeptide.

Expression and cloning will typically contain a promoter that is recognized by the host organism and operably linked to the molecule encoding an antigen binding protein that specifically binds to PCSK9. Promoters are untranscribed sequences located upstream (i.e., 5′) to the start codon of a structural gene (generally within about 100 to 1000 bp) that control transcription of the structural gene. Promoters are conventionally grouped into one of two classes: inducible promoters and constitutive promoters. Inducible promoters initiate increased levels of transcription from DNA under their control in response to some change in culture conditions, such as the presence or absence of a nutrient or a change in temperature. Constitutive promoters, on the other hand, uniformly transcribe a gene to which they are operably linked, that is, with little or no control over gene expression. A large number of promoters, recognized by a variety of potential host cells, are well known. A suitable promoter is operably linked to the DNA encoding heavy chain or light chain comprising an antigen binding protein by removing the promoter from the source DNA by restriction enzyme digestion and inserting the desired promoter sequence into the vector.

Suitable promoters for use with yeast hosts are also well known in the art. Yeast enhancers are advantageously used with yeast promoters. Suitable promoters for use with mammalian host cells are well known and include, but are not limited to, those obtained from the genomes of viruses such as polyoma virus, fowlpox virus, adenovirus (such as Adenovirus 2), bovine papilloma virus, avian sarcoma virus, cytomegalovirus, retroviruses, hepatitis-B virus, and Simian Virus 40 (SV40). Other suitable mammalian promoters include heterologous mammalian promoters, for example, heat-shock promoters and the actin promoter.

Additional promoters which can be of interest include, but are not limited to: SV40 early promoter (Benoist & Chambon, (1981) Nature 290:304-310); CMV promoter (Thomsen et al., (1984) Proc. Natl. Acad. U.S.A. 81:659-663); the promoter contained in the 3′ long terminal repeat of Rous sarcoma virus (Yamamoto et al., (1980) Cell 22:787-97); herpes thymidine kinase promoter (Wagner et al., (1981) Proc. Natl. Acad. Sci. U.S.A. 78:1444-45); promoter and regulatory sequences from the metallothionine gene (Prinster et al., (1982) Nature 296:39-42); and prokaryotic promoters such as the beta-lactamase promoter (Villa-Kamaroff et al., (1978) Proc. Natl. Acad. Sci. U.S.A. 75:3727-31); or the tac promoter (DeBoer et al., (1983) Proc. Natl. Acad. Sci. U.S.A. 80:21-25). Also of interest are the following animal transcriptional control regions, which exhibit tissue specificity and have been utilized in transgenic animals: the elastase I gene control region that is active in pancreatic acinar cells (Swift et al., (1984) Cell 38:639-46; Ornitz et al., (1986) Cold Spring Harbor Symp. Quant. Biol. 50:399-409; MacDonald, (1987) Hepatology 7:425-515); the insulin gene control region that is active in pancreatic beta cells (Hanahan, (1985) Nature 315:115-22); the immunoglobulin gene control region that is active in lymphoid cells (Grosschedl et al., (1984) Cell 38:647-58; Adames et al., (1985) Nature 318:533-38; Alexander et al., (1987) Mol. Cell. Biol. 7:1436-44); the mouse mammary tumor virus control region that is active in testicular, breast, lymphoid and mast cells (Leder et al., (1986) Cell 45:485-95); the albumin gene control region that is active in liver (Pinkert et al., (1987) Genes and Devel. 1:268-76); the alpha-feto-protein gene control region that is active in liver (Krumlauf et al., (1985) Mol. Cell. Biol. 5:1639-48; Hammer et al., (1987) Science 253:53-58); the alpha 1-antitrypsin gene control region that is active in liver (Kelsey et al., (1987) Genes and Devel. 1:161-71); the beta-globin gene control region that is active in myeloid cells (Mogram et al., (1985) Nature 315:338-40; Kollias et al., (1986) Cell 46:89-94); the myelin basic protein gene control region that is active in oligodendrocyte cells in the brain (Readhead et al., (1987) Cell 48:703-12); the myosin light chain-2 gene control region that is active in skeletal muscle (Sani, (1985) Nature 314:283-86); and the gonadotropic releasing hormone gene control region that is active in the hypothalamus (Mason et al., (1986) Science 234:1372-78).

An enhancer sequence can be inserted into the vector to increase transcription of DNA encoding light chain or heavy chain comprising an antigen binding protein that specifically binds to PCSK9 by higher eukaryotes, e.g., a human antigen binding protein that specifically binds to PCSK9. Enhancers are cis-acting elements of DNA, usually about 10-300 bp in length, that act on the promoter to increase transcription. Enhancers are relatively orientation and position independent, having been found at positions both 5′ and 3′ to the transcription unit. Several enhancer sequences available from mammalian genes are known (e.g., globin, elastase, albumin, alpha-feto-protein and insulin). Typically, however, an enhancer from a virus is used. The SV40 enhancer, the cytomegalovirus early promoter enhancer, the polyoma enhancer, and adenovirus enhancers known in the art are exemplary enhancing elements for the activation of eukaryotic promoters. While an enhancer can be positioned in the vector either 5′ or 3′ to a coding sequence, it is typically located at a site 5′ from the promoter. A sequence encoding an appropriate native or heterologous signal sequence (leader sequence or signal peptide) can be incorporated into an expression vector, to promote extracellular secretion of the antibody. The choice of signal peptide or leader depends on the type of host cells in which the antibody is to be produced, and a heterologous signal sequence can replace the native signal sequence. Examples of signal peptides that are functional in mammalian host cells include the following: the signal sequence for interleukin-7 (IL-7) described in U.S. Pat. No. 4,965,195; the signal sequence for interleukin-2 receptor described in Cosman et al., (1984) Nature 312:768-71; the interleukin-4 receptor signal peptide described in EP Patent No. 0367 566; the type I interleukin-1 receptor signal peptide described in U.S. Pat. No. 4,968,607; the type II interleukin-1 receptor signal peptide described in EP Patent No. 0 460 846.

Expression vectors can be constructed from a starting vector such as a commercially available vector. Such vectors can but need not contain all of the desired flanking sequences. Where one or more of the flanking sequences are not already present in the vector, they can be individually obtained and ligated into the vector. Methods used for obtaining each of the flanking sequences are well known to one skilled in the art.

After the vector has been constructed and a nucleic acid molecule encoding light chain, a heavy chain, or a light chain and a heavy chain comprising an antigen binding protein that specifically binds to PCSK9 has been inserted into the proper site of the vector, the completed vector can be inserted into a suitable host cell for amplification and/or polypeptide expression. The transformation of an expression vector for an antigen binding protein into a selected host cell can be accomplished by well known methods including transfection, infection, calcium phosphate co-precipitation, electroporation, microinjection, lipofection, DEAE-dextran mediated transfection, or other known techniques. The method selected will in part be a function of the type of host cell to be used. These methods and other suitable methods are well known to the skilled artisan, and are set forth, for example, in Sambrook et al., (2001), supra.

A host cell, when cultured under appropriate conditions, synthesizes an antigen binding protein that can subsequently be collected from the culture medium (if the host cell secretes it into the medium) or directly from the host cell producing it (if it is not secreted). The selection of an appropriate host cell will depend upon various factors, such as desired expression levels, polypeptide modifications that are desirable or necessary for activity (such as glycosylation or phosphorylation) and ease of folding into a biologically active molecule.

Mammalian cell lines available as hosts for expression are well known in the art and include, but are not limited to, immortalized cell lines available from the American Type Culture Collection (ATCC), including but not limited to HeLa cells, Human Embryonic Kidney 293 cells (HEK293 cells), Chinese hamster ovary (CHO) cells, HeLa cells, baby hamster kidney (BHK) cells, monkey kidney cells (COS), human hepatocellular carcinoma cells (e.g., Hep G2), and a number of other cell lines. In certain embodiments, cell lines can be selected through determining which cell lines have high expression levels and constitutively produce antigen binding proteins with desirable binding properties (e.g., the ability to bind PCSK9). In another embodiment, a cell line from the B cell lineage that does not make its own antibody but has a capacity to make and secrete a heterologous antibody can be selected. The ability to inhibit PCSK9 binding to LDLR can also form a selection criterion.

### Uses of Antigen Binding Proteins for Diagnostic and Therapeutic Purposes

In certain instances, PCSK9 activity correlates with a number of human disease states. For example, in certain instances, too much PCSK9 activity correlates with certain conditions, such as hypercholesterolemia. Therefore, in certain instances, modulating PCSK9 activity can be therapeutically useful. In certain embodiments, a neutralizing antigen binding protein to PCSK9 is used to modulate at least one PCSK9 activity (e.g., binding to LDLR). Such methods can treat and/or prevent and/or reduce the risk of disorders that relate to elevated serum cholesterol levels or in which elevated cholesterol levels are relevant.

As will be appreciated by one of skill in the art, in light of the present disclosure, disorders that relate to, involve, or can be influenced by varied cholesterol, LDL, or LDLR levels can be addressed by various embodiments of the antigen binding proteins. In some embodiments, a “cholesterol related disorder” (which includes “serum cholesterol related disorders”) includes any one or more of the following: hypercholesterolemia, heart disease, metabolic syndrome, diabetes, coronary heart disease, stroke, cardiovascular diseases, Alzheimer's disease and generally dyslipidemias, which can be manifested, for example, by an elevated total serum cholesterol, elevated LDL, elevated triglycerides, elevated VLDL, and/or low HDL. Some non-limiting examples of primary and secondary dyslipidemias that can be treated using an ABP, either alone, or in combination with one or more other agents include the metabolic syndrome, diabetes mellitus, familial combined hyperlipidemia, familial hypertriglyceridemia, familial hypercholesterolemias, including heterozygous hypercholesterolemia, homozygous hypercholesterolemia, familial defective apolipoprotein B-100; polygenic hypercholesterolemia; remnant removal disease, hepatic lipase deficiency; dyslipidemia secondary to any of the following: dietary indiscretion, hypothyroidism, drugs including estrogen and progestin therapy, beta-blockers, and thiazide diuretics; nephrotic syndrome, chronic renal failure, Cushing's syndrome, primary biliary cirrhosis, glycogen storage diseases, hepatoma, cholestasis, acromegaly, insulinoma, isolated growth hormone deficiency, mTOR inhibitors, HIV protease inhibitors, certain anti-depressants, and alcohol-induced hypertriglyceridemia. ABP can also be useful in preventing or treating atherosclerotic diseases, such as, for example, coronary heart disease, coronary artery disease, peripheral arterial disease, stroke (ischemic and hemorrhagic), angina pectoris, or cerebrovascular disease and acute coronary syndrome, myocardial infarction. In some embodiments, the ABP is useful in reducing the risk of: nonfatal heart attacks, fatal and nonfatal strokes, certain types of heart surgery, hospitalization for heart failure, chest pain in patients with heart disease, and/or cardiovascular events because of established heart disease such as prior heart attack, prior heart surgery, and/or chest pain with evidence of clogged arteries. In some embodiments, the ABP and methods can be used to reduce the risk of recurrent cardiovascular events.

As will be appreciated by one of skill in the art, diseases or disorders that are generally addressable (either treatable or preventable) through the use of statins can also benefit from the application of the instant antigen binding proteins. In addition, in some embodiments, disorders or disease that can benefit from increased LDLR expression can also be treated by various embodiments of the antigen binding proteins. In addition, as will be appreciated by one of skill in the art, the use of the anti-PCSK9 antibodies can be especially useful in the treatment of diabetes. Not only is diabetes a risk factor for coronary heart disease, but insulin increases the expression of PCSK9. That is, people with diabetes have elevated plasma lipid levels (which can be related to high PCSK9 levels) and can benefit from lowering those levels. This is generally discussed in more detail in Costet et al. (“Hepatic PCSK9 Expression is Regulated by Nutritional Status via Insulin and Sterol Regulatory Element-binding Protein 1C”, J. Biol. Chem., 281: 6211-6218, 2006), the entirety of which is incorporated herein by reference.

In some embodiments, the antigen binding protein is administered to those who have diabetes mellitus, abdominal aortic aneurysm, atherosclerosis and/or peripheral vascular disease in order to decrease their serum cholesterol levels to a safer range. In some embodiments, the antigen binding protein is administered to patients at risk of developing any of the herein described disorders. In some embodiments, the ABPs are administered to subjects that smoke, have hypertension or a familial history of early heart attacks.

In some embodiments, a subject is administered an ABP if they are at a moderate risk or higher on the 2004 NCEP treatment goals. In some embodiments, the ABP is administered to a subject if the subject's LDL cholesterol level is greater than 160 mg/dl. In some embodiments, the ABP is administered if the subjects LDL cholesterol level is greater than 130 mg/dl (and they have a moderate or moderately high risk according to the 2004 NCEP treatment goals). In some embodiments, the ABP is administered if the subjects LDL cholesterol level is greater than 100 mg/dl (and they have a high or very high risk according to the 2004 NCEP treatment goals).

A physician will be able to select an appropriate treatment indication and target lipid level depending on the individual profile of a particular patient. One well-accepted standard for guiding treatment of hyperlipidemia is the Third Report of the National Cholesterol Education Program (NCEP) Expert Panel on Detection, Evaluation, and Treatment of the High Blood Cholesterol in Adults (Adult Treatment Panel III) Final Report, National Institutes of Health, NIH Publication No. 02-5215 (2002), the printed publication of which is hereby incorporated by reference in its entirety.

In some embodiments, antigen binding proteins to PCSK9 are used to decrease the amount of PCSK9 activity from an abnormally high level or even a normal level. In some embodiments, antigen binding proteins to PCSK9 are used to treat or prevent hypercholesterolemia and/or in the preparation of medicaments therefore and/or for other cholesterol related disorders (such as those noted herein). In certain embodiments, an antigen binding protein to PCSK9 is used to treat or prevent conditions such as hypercholesterolemia in which PCSK9 activity is normal. In such conditions, for example, reduction of PCSK9 activity to below normal can provide a therapeutic effect.

In some embodiments, more than one antigen binding protein to PCSK9 is used to modulate PCSK9 activity.

In certain embodiments, methods are provided of treating a cholesterol related disorder, such as hypercholesterolemia comprising administering a therapeutically effective amount of one or more antigen binding proteins to PCSK9 and another therapeutic agent.

In certain embodiments, an antigen binding protein to PCSK9 is administered alone. In certain embodiments, an antigen binding protein to PCSK9 is administered prior to the administration of at least one other therapeutic agent. In certain embodiments, an antigen binding protein to PCSK9 is administered concurrent with the administration of at least one other therapeutic agent. In certain embodiments, an antigen binding protein to PCSK9 is administered subsequent to the administration of at least one other therapeutic agent. In other embodiments, an antigen binding protein to PCSK9 is administered prior to the administration of at least one other therapeutic agent. Therapeutic agents (apart from the antigen binding protein), include, but are not limited to, at least one other cholesterol-lowering (serum and/or total body cholesterol) agent or an agent. In some embodiments, the agent increases the expression of LDLR, have been observed to increase serum HDL levels, lower LDL levels or lower triglyceride levels. Exemplary therapeutic agents include, but are not limited to, statins (atorvastatin, cerivastatin, fluvastatin, lovastatin, mevastatin, pitavastatin, pravastatin, rosuvastatin, simvastatin), Nicotinic acid (Niacin) (NIACOR, Niaspan (slow release niacin), Slo-Niacin (slow release niacin)), Fibric acid (Lopid (Gemfibrozil), Tricor (fenofibrate), Bile acid sequestrants (Questran (cholestyramine), colesevelam (Welchol), Colestid (colestipol)), Cholesterol absorption inhibitors (Zetia (ezetimibe)), Combining nicotinic acid with statin (Advicor (lovastatin and niaspan), Combining a statin with an absorption inhibitor (Vytorin (Zocor and Zetia) and/or lipid modifying agents. In some embodiments, the ABP is combined with PPAR gamma agonsits, PPAR alpha/gamma agonists, squalene synthase inhibitors, CETP inhibitors, anti-hypertensives, anti-diabetic agents (such as sulphonyl ureas, insulin, GLP-1 analogs, DDPIV inhibitors), ApoB modulators, MTP inhibitoris and/or arteriosclerosis obliterans treatments. In some embodiments, the ABP is combined with an agent that increases the level of LDLR protein in a subject, such as statins, certain cytokines like oncostatin M, estrogen, and/or certain herbal ingredients such as berberine. In some embodiments, the ABP is combined with an agent that increases serum cholesterol levels in a subject (such as certain anti-psycotic agents, certain HIV protease inhibitors, dietary factors such as high fructose, sucrose, cholesterol or certain fatty acids and certain nuclear receptor agonists and antagonists for RXR, RAR, LXR, FXR). In some embodiments, the ABP is combined with an agent that increases the level of PCSK9 in a subject, such as statins and/or insulin. The combination of the two can allow for the undesirable side-effects of other agents to be mitigated by the ABP. As will be appreciated by one of skill in the art, in some embodiments, the ABP is combined with the other agent/compound. In some embodiments, the ABP and other agent are administered concurrently. In some embodiments, the ABP and other agent are not administered simultaneously, with the ABP being administered before or after the agent is administered. In some embodiments, the subject receives both the ABP and the other agent (that increases the level of LDLR) during a same period of prevention, occurrence of a disorder, and/or period of treatment.

Pharmaceutical compositions of the invention can be administered in combination therapy, i.e., combined with other agents. In certain embodiments, the combination therapy comprises an antigen binding protein capable of binding PCSK9, in combination with at least one anti-cholesterol agent. Agents include, but are not limited to, in vitro synthetically prepared chemical compositions, antibodies, antigen binding regions, and combinations and conjugates thereof. In certain embodiments, an agent can act as an agonist, antagonist, allosteric modulator, or toxin. In certain embodiments, an agent can act to inhibit or stimulate its target (e.g., receptor or enzyme activation or inhibition), and thereby promote increased expression of LDLR or decrease serum cholesterol levels.

In certain embodiments, an antigen binding protein to PCSK9 can be administered prior to, concurrent with, and subsequent to treatment with a cholesterol-lowering (serum and/or total cholesterol) agent. In certain embodiments, an antigen binding protein to PCSK9 can be administered prophylactically to prevent or mitigate the onset of hypercholesterolemia, heart disease, diabetes, and/or any of the cholesterol related disorder. In certain embodiments, an antigen binding protein to PCSK9 can be administered for the treatment of an existing hypercholesterolemia condition. In some embodiments, the ABP delays the onset of the disorder and/or symptoms associated with the disorder. In some embodiments, the ABP is provided to a subject lacking any symptoms of any one of the cholesterol related disorders or a subset thereof.

In certain embodiments, an antigen binding protein to PCSK9 is used with particular therapeutic agents to treat various cholesterol related disorders, such as hypercholesterolemia. In certain embodiments, in view of the condition and the desired level of treatment, two, three, or more agents can be administered. In certain embodiments, such agents can be provided together by inclusion in the same formulation. In certain embodiments, such agent(s) and an antigen binding protein to PCSK9 can be provided together by inclusion in the same formulation. In certain embodiments, such agents can be formulated separately and provided together by inclusion in a treatment kit. In certain embodiments, such agents and an antigen binding protein to PCSK9 can be formulated separately and provided together by inclusion in a treatment kit. In certain embodiments, such agents can be provided separately. In certain embodiments, when administered by gene therapy, the genes encoding protein agents and/or an antigen binding protein to PCSK9 can be included in the same vector. In certain embodiments, the genes encoding protein agents and/or an antigen binding protein to PCSK9 can be under the control of the same promoter region. In certain embodiments, the genes encoding protein agents and/or an antigen binding protein to PCSK9 can be in separate vectors.

In certain embodiments, the invention provides for pharmaceutical compositions comprising an antigen binding protein to PCSK9 together with a pharmaceutically acceptable diluent, carrier, solubilizer, emulsifier, preservative and/or adjuvant.

In certain embodiments, the invention provides for pharmaceutical compositions comprising an antigen binding protein to PCSK9 and a therapeutically effective amount of at least one additional therapeutic agent, together with a pharmaceutically acceptable diluent, carrier, solubilizer, emulsifier, preservative and/or adjuvant.

In certain embodiments, an antigen binding protein to PCSK9 can be used with at least one therapeutic agent for inflammation. In certain embodiments, an antigen binding protein to PCSK9 can be used with at least one therapeutic agent for an immune disorder. Exemplary therapeutic agents for inflammation and immune disorders include, but are not limited to cyclooxygenase type 1 (COX-1) and cyclooxygenase type 2 (COX-2) inhibitors small molecule modulators of 38 kDa mitogen-activated protein kinase (p38-MAPK); small molecule modulators of intracellular molecules involved in inflammation pathways, wherein such intracellular molecules include, but are not limited to, jnk, IKK, NF-κB, ZAP70, and lck. Certain exemplary therapeutic agents for inflammation are described, e.g., in C. A. Dinarello & L. L. Moldawer Proinflammatory and Anti-Inflammatory Cytokines in Rheumatoid Arthritis: A Primer for Clinicians Third Edition (2001) Amgen Inc. Thousand Oaks, Calif.

In certain embodiments, pharmaceutical compositions will include more than one different antigen binding protein to PCSK9. In certain embodiments, pharmaceutical compositions will include more than one antigen binding protein to PCSK9 wherein the antigen binding proteins to PCSK9 bind more than one epitope. In some embodiments, the various antigen binding proteins will not compete with one another for binding to PCSK9. In some embodiments, any of the antigen binding proteins depicted in Table 1-4, can be combined together in a pharmaceutical composition.

In certain embodiments, acceptable formulation materials preferably are nontoxic to recipients at the dosages and concentrations employed. In some embodiments, the formulation material(s) are for s.c. and/or I.V. administration. In certain embodiments, the pharmaceutical composition can contain formulation materials for modifying, maintaining or preserving, for example, the pH, osmolarity, viscosity, clarity, color, isotonicity, odor, sterility, stability, rate of dissolution or release, adsorption or penetration of the composition. In certain embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. (Remington's Pharmaceutical Sciences, 18^{th} Edition, A. R. Gennaro, ed., Mack Publishing Company (1995). In some embodiments, the formulation comprises PBS; 20 mM NaOAC, pH 5.2, 50 mM NaCl; and/or 10 mM NAOAC, pH 5.2, 9% Sucrose.

In certain embodiments, an antigen binding protein to PCSK9 and/or a therapeutic molecule is linked to a half-life extending vehicle known in the art. Such vehicles include, but are not limited to, polyethylene glycol, glycogen (e.g., glycosylation of the ABP), and dextran. Such vehicles are described, e.g., in U.S. application Ser. No. 09/428,082, now U.S. Pat. No. 6,660,843 and published PCT Application No. WO 99/25044, which are hereby incorporated by reference for any purpose.

In certain embodiments, the optimal pharmaceutical composition will be determined by one skilled in the art depending upon, for example, the intended route of administration, delivery format and desired dosage. See, for example, Remington's Pharmaceutical Sciences, supra. In certain embodiments, such compositions may influence the physical state, stability, rate of in vivo release and rate of in vivo clearance of the antibodies of the invention.

In certain embodiments, the primary vehicle or carrier in a pharmaceutical composition can be either aqueous or non-aqueous in nature. For example, in certain embodiments, a suitable vehicle or carrier can be water for injection, physiological saline solution or artificial cerebrospinal fluid, possibly supplemented with other materials common in compositions for parenteral administration. In some embodiments, the saline comprises isotonic phosphate-buffered saline. In certain embodiments, neutral buffered saline or saline mixed with serum albumin are further exemplary vehicles. In certain embodiments, a composition comprising an antigen binding protein to PCSK9, with or without at least one additional therapeutic agents, can be prepared for storage by mixing the selected composition having the desired degree of purity with optional formulation agents (Remington's Pharmaceutical Sciences, supra) in the form of a lyophilized cake or an aqueous solution. Further, in certain embodiments, a composition comprising an antigen binding protein to PCSK9, with or without at least one additional therapeutic agent, can be formulated as a lyophilizate using appropriate excipients such as sucrose.

In certain embodiments, the pharmaceutical composition can be selected for parenteral delivery. In certain embodiments, the compositions can be selected for inhalation or for delivery through the digestive tract, such as orally. The preparation of such pharmaceutically acceptable compositions is within the ability of one skilled in the art.

In certain embodiments, the formulation components are present in concentrations that are acceptable to the site of administration. In certain embodiments, buffers are used to maintain the composition at physiological pH or at a slightly lower pH, typically within a pH range of from about 5 to about 8.

In certain embodiments, when parenteral administration is contemplated, a therapeutic composition can be in the form of a pyrogen-free, parenterally acceptable aqueous solution comprising a desired antigen binding protein to PCSK9, with or without additional therapeutic agents, in a pharmaceutically acceptable vehicle. In certain embodiments, a vehicle for parenteral injection is sterile distilled water in which an antigen binding protein to PCSK9, with or without at least one additional therapeutic agent, is formulated as a sterile, isotonic solution, properly preserved. In certain embodiments, the preparation can involve the formulation of the desired molecule with an agent, such as injectable microspheres, bio-erodible particles, polymeric compounds (such as polylactic acid or polyglycolic acid), beads or liposomes, that can provide for the controlled or sustained release of the product which can then be delivered via a depot injection. In certain embodiments, hyaluronic acid can also be used, and can have the effect of promoting sustained duration in the circulation. In certain embodiments, implantable drug delivery devices can be used to introduce the desired molecule.

In certain embodiments, a pharmaceutical composition can be formulated for inhalation. In certain embodiments, an antigen binding protein to PCSK9, with or without at least one additional therapeutic agent, can be formulated as a dry powder for inhalation. In certain embodiments, an inhalation solution comprising an antigen binding protein to PCSK9, with or without at least one additional therapeutic agent, can be formulated with a propellant for aerosol delivery. In certain embodiments, solutions can be nebulized. Pulmonary administration is further described in PCT application no. PCT/US94/001875, which describes pulmonary delivery of chemically modified proteins.

In certain embodiments, it is contemplated that formulations can be administered orally. In certain embodiments, an antigen binding protein to PCSK9, with or without at least one additional therapeutic agents, that is administered in this fashion can be formulated with or without those carriers customarily used in the compounding of solid dosage forms such as tablets and capsules. In certain embodiments, a capsule can be designed to release the active portion of the formulation at the point in the gastrointestinal tract when bioavailability is maximized and pre-systemic degradation is minimized. In certain embodiments, at least one additional agent can be included to facilitate absorption of an antigen binding protein to PCSK9 and/or any additional therapeutic agents. In certain embodiments, diluents, flavorings, low melting point waxes, vegetable oils, lubricants, suspending agents, tablet disintegrating agents, and binders can also be employed.

In certain embodiments, a pharmaceutical composition can involve an effective quantity of an antigen binding protein to PCSK9, with or without at least one additional therapeutic agents, in a mixture with non-toxic excipients which are suitable for the manufacture of tablets. In certain embodiments, by dissolving the tablets in sterile water, or another appropriate vehicle, solutions can be prepared in unit-dose form. In certain embodiments, suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions will be evident to those skilled in the art, including formulations involving antigen binding proteins to PCSK9, with or without at least one additional therapeutic agent(s), in sustained- or controlled-delivery formulations. In certain embodiments, techniques for formulating a variety of other sustained- or controlled-delivery means, such as liposome carriers, bio-erodible microparticles or porous beads and depot injections, are also known to those skilled in the art. See for example, PCT Application No. PCT/US93/00829 which describes the controlled release of porous polymeric microparticles for the delivery of pharmaceutical compositions. In certain embodiments, sustained-release preparations can include semipermeable polymer matrices in the form of shaped articles, e.g. films, or microcapsules. Sustained release matrices can include polyesters, hydrogels, polylactides (U.S. Pat. No. 3,773,919 and EP 058,481), copolymers of L-glutamic acid and gamma ethyl-L-glutamate (Sidman et al., Biopolymers, 22:547-556 (1983)), poly (2-hydroxyethyl-methacrylate) (Langer et al., J. Biomed. Mater. Res., 15:167-277 (1981) and Langer, Chem. Tech., 12:98-105 (1982)), ethylene vinyl acetate (Langer et al., supra) or poly-D(−)-3-hydroxybutyric acid (EP 133,988). In certain embodiments, sustained release compositions can also include liposomes, which can be prepared by any of several methods known in the art. See, e.g., Eppstein et al., Proc. Natl. Acad. Sci. USA, 82:3688-3692 (1985); EP 036,676; EP 088,046 and EP 143,949.

The pharmaceutical composition to be used for in vivo administration typically is sterile. In certain embodiments, this can be accomplished by filtration through sterile filtration membranes. In certain embodiments, where the composition is lyophilized, sterilization using this method can be conducted either prior to or following lyophilization and reconstitution. In certain embodiments, the composition for parenteral administration can be stored in lyophilized form or in a solution. In certain embodiments, parenteral compositions generally are placed into a container having a sterile access port, for example, an intravenous solution bag or vial having a stopper pierceable by a hypodermic injection needle.

In certain embodiments, once the pharmaceutical composition has been formulated, it can be stored in sterile vials as a solution, suspension, gel, emulsion, solid, or as a dehydrated or lyophilized powder. In certain embodiments, such formulations can be stored either in a ready-to-use form or in a form (e.g., lyophilized) that is reconstituted prior to administration.

In certain embodiments, kits are provided for producing a single-dose administration unit. In certain embodiments, the kit can contain both a first container having a dried protein and a second container having an aqueous formulation. In certain embodiments, kits containing single and multi-chambered pre-filled syringes (e.g., liquid syringes and lyosyringes) are included.

In certain embodiments, the effective amount of a pharmaceutical composition comprising an antigen binding protein to PCSK9, with or without at least one additional therapeutic agent, to be employed therapeutically will depend, for example, upon the therapeutic context and objectives. One skilled in the art will appreciate that the appropriate dosage levels for treatment, according to certain embodiments, will thus vary depending, in part, upon the molecule delivered, the indication for which an antigen binding protein to PCSK9, with or without at least one additional therapeutic agent, is being used, the route of administration, and the size (body weight, body surface or organ size) and/or condition (the age and general health) of the patient. In certain embodiments, the clinician can titer the dosage and modify the route of administration to obtain the optimal therapeutic effect. In certain embodiments, a typical dosage can range from about 0.1 μg/kg to up to about 100 mg/kg or more, depending on the factors mentioned above.

In certain embodiments, the frequency of dosing will take into account the pharmacokinetic parameters of an antigen binding protein to PCSK9 and/or any additional therapeutic agents in the formulation used. In certain embodiments, a clinician will administer the composition until a dosage is reached that achieves the desired effect. In certain embodiments, the composition can therefore be administered as a single dose, or as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. In certain embodiments, appropriate dosages can be ascertained through use of appropriate dose-response data. In some embodiments, the amount and frequency of administration can take into account the desired cholesterol level (serum and/or total) to be obtained and the subject's present cholesterol level, LDL level, and/or LDLR levels, all of which can be obtained by methods that are well known to those of skill in the art.

In certain embodiments, the route of administration of the pharmaceutical composition is in accord with known methods, e.g. orally, through injection by intravenous, intraperitoneal, intracerebral (intra-parenchymal), intracerebroventricular, intramuscular, subcutaneously, intra-ocular, intraarterial, intraportal, or intralesional routes; by sustained release systems or by implantation devices. In certain embodiments, the compositions can be administered by bolus injection or continuously by infusion, or by implantation device.

In certain embodiments, the composition can be administered locally via implantation of a membrane, sponge or another appropriate material onto which the desired molecule has been absorbed or encapsulated. In certain embodiments, where an implantation device is used, the device can be implanted into any suitable tissue or organ, and delivery of the desired molecule can be via diffusion, timed-release bolus, or continuous administration.

In certain embodiments, it can be desirable to use a pharmaceutical composition comprising an antigen binding protein to PCSK9, with or without at least one additional therapeutic agent, in an ex vivo manner. In such instances, cells, tissues and/or organs that have been removed from the patient are exposed to a pharmaceutical composition comprising an antigen binding protein to PCSK9, with or without at least one additional therapeutic agent, after which the cells, tissues and/or organs are subsequently implanted back into the patient.

In certain embodiments, an antigen binding protein to PCSK9 and/or any additional therapeutic agents can be delivered by implanting certain cells that have been genetically engineered, using methods such as those described herein, to express and secrete the polypeptides. In certain embodiments, such cells can be animal or human cells, and can be autologous, heterologous, or xenogeneic. In certain embodiments, the cells can be immortalized. In certain embodiments, in order to decrease the chance of an immunological response, the cells can be encapsulated to avoid infiltration of surrounding tissues. In certain embodiments, the encapsulation materials are typically biocompatible, semi-permeable polymeric enclosures or membranes that allow the release of the protein product(s) but prevent the destruction of the cells by the patient's immune system or by other detrimental factors from the surrounding tissues.

Based on the ability of ABPs to significantly neutralize PCSK9 activity (as demonstrated in the Examples below), these ABPs will have therapeutic effects in treating and preventing symptoms and conditions resulting from PCSK9-mediated activity, such as hypercholesterolemia.

Diagnostic Applications

In some embodiments, the ABP is used as a diagnostic tool. The ABP can be used to assay the amount of PCSK9 present in a sample and/or subject. As will be appreciated by one of skill in the art, such ABPs need not be neutralizing ABPs. In some embodiments, the diagnostic ABP is not a neutralizing ABP. In some embodiments, the diagnostic ABP binds to a different epitope than the neutralizing ABP binds to. In some embodiments, the two ABPs do not compete with one another.

In some embodiments, the ABPs disclosed herein are used or provided in an assay kit and/or method for the detection of PCSK9 in mammalian tissues or cells in order to screen/diagnose for a disease or disorder associated with changes in levels of PCSK9. The kit comprises an ABP that binds PCSK9 and means for indicating the binding of the ABP with PCSK9, if present, and optionally PCSK9 protein levels. Various means for indicating the presence of an ABP can be used. For example, fluorophores, other molecular probes, or enzymes can be linked to the ABP and the presence of the ABP can be observed in a variety of ways. The method for screening for such disorders can involve the use of the kit, or simply the use of one of the disclosed ABPs and the determination of whether the ABP binds to PCSK9 in a sample. As will be appreciated by one of skill in the art, high or elevated levels of PCSK9 will result in larger amounts of the ABP binding to PCSK9 in the sample. Thus, degree of ABP binding can be used to determine how much PCSK9 is in a sample. Subjects or samples with an amount of PCSK9 that is greater than a predetermined amount (e.g., an amount or range that a person without a PCSK9 related disorder would have) can be characterized as having a PCSK9 mediated disorder. In some embodiments, the ABP is administered to a subject taking a statin, in order to determine if the statin has increased the amount of PCSK9 in the subject.

In some embodiments, the ABP is a non-neutralizing ABP and is used to determine the amount of PCSK9 in a subject receiving an ABP and/or statin treatment.

### EXAMPLES

The following examples, including the experiments conducted and the results achieved, are provided for illustrative purposes only and are not to be construed as limiting.

### Example 1

### Generation of Anti-PCSK9 Antibodies and Hybridomas

Antibodies to the self-cleaved, mature, secreted PCSK9 comprising amino acids 31 to 692 of the amino acid sequence of SEQ ID NO: 2, were raised in XenoMouse® mice (Abgenix, Fremont, Calif.), which are mice containing human immunoglobulin genes. XenoMouse® strains including; XMG2KL, XMG4KL, XMG2/K and XMG4/K were used for immunization. PCSK9 was prepared using standard recombinant techniques using the GenBank sequence as reference (NM_{—}174936).

Each mouse was injected with a total of 10 μg of antigen delivered intraperitoneally into the abdomen. Subsequent boosts were 5 ug doses and injection method was staggered between intraperitoneal injections into the abdomen and sub-cutaneous injections at the base of the tail. For intraperitoneal injections, antigen was prepared as an emulsion with TiterMax® Gold (Sigma, Cat # T2684) and for subcutaneous injections antigen was mixed with Alum (aluminum phosphate) and CpG oligos. A final injection of 5 μg of antigen per mouse was delivered in Phospho buffered saline (“PBS”) and delivered into 2 sites, 50% IP into the abdomen and 50% SQ at the base of tail. The mice were injected with antigen eight to eleven times.

Mice were then monitored for an anti-PCSK9 specific immune response using a titer protocol as follows: Costar 3368 medium binding plates were coated with neutravadin at 8 ug/ml (50 ul/well) and incubated at 4° C. in 1×PBS/0.05% azide overnight. They were washed using TiterTek 3-cycle wash with reversed osmosis purified (“RO”) water. Plates were blocked using 250 ul of 1×PBS/1% milk and incubated for at least 30 minutes at room temperature (“RT”). Block was washed off using TiterTek 3-cycle wash with RO water. Biotinylated (b)-human PCSK9 was captured at 2 ug/ml in 1×PBS/1% milk/10 mM Ca2+(assay diluent) 50 ul/well and incubated for 1 hr at RT. Unbound b-PCSK9 was washed off using TiterTek 3-cycle wash with RO water. For the primary antibody, sera was titrated 1:3 in duplicate from 1:100. This was done in assay diluent 50 ul/well and incubated for 1 hr at RT and then washed using TiterTek 3-cycle wash with RO water. The secondary antibody was goat anti Human IgG Fc HRP at 400 ng/ml in assay diluent at 50 ul/well. This was incubated for 1 hr at RT. This was then washed using TiterTek 3-cycle wash with RO water and patted dry on paper towels. For the substrate, one-step TMB solution (Neogen, Lexington, Ky.) was used (50 ul/well) and it was allowed to develop for 30 min at RT. Positive controls to detect plate bound PCSK9 were soluble LDL receptor (R&D Systems, Cat #2148LD/CF) and a polyclonal rabbit anti-PCSK9 antibody (Caymen Chemical #10007185) titrated 1:3 in duplicate from 3 μg/ml in assay diluent. LDLR was detected with goat anti LDLR (R&D Systems, Cat #AF2148) and rabbit anti goat IgGFc HRP at a concentration of 400 ng/ml; the rabbit polyclonal was detected with goat anti-rabbit IgG Fc at a concentration of 400 ng/ml in assay diluent. The negative control was naive XMG2-KL and XMG4-KL sera titrated 1:3 in duplicate from 1:100 in assay diluent.

Titers of the antibody against human PCSK9 were tested by ELISA assay for mice immunized with soluble antigen as described. Animals which were identified to have raised specific immune responses to PCSK9 were harvested and advanced to antibody generation.

Multiple rounds of antibody generation were conducted to generate the panels used to select the antibodies described herein.

### Example 2

### Recovery of Lymphocytes, B-Cell Isolations, Fusions and Generation of Hybridomas

This example outlines how the immune cells were recovered and the hybridomas were generated. Selected immunized mice were sacrificed by cervical dislocation and the draining lymph nodes were harvested and pooled from each cohort. The B cells were dissociated from lymphoid tissue by grinding in DMEM to release the cells from the tissues, and the cells were suspended in DMEM. The cells were counted, and 0.9 ml DMEM per 100 million lymphocytes was added to the cell pellet to resuspend the cells gently but completely.

Lymphocytes were mixed with nonsecretory myeloma P3X63Ag8.653 cells purchased from ATCC, cat.# CRL 1580 (Kearney et al., (1979) J. Immunol. 123, 1548-1550) at a ratio of 1:4. The cell mixture was gently pelleted by centrifugation at 400×g 4 min. After decanting of the supernatant, the cells were gently mixed using a 1 ml pipette. Preheated PEG/DMSO solution from Sigma (cat# P7306) (1 ml per million of B-cells) was slowly added with gentle agitation over 1 min followed by 1 min of mixing. Preheated IDMEM (2 ml per million of B cells) (DMEM without glutamine, L-glutamine, pen/strep, MEM non-essential amino acids (all from Invitrogen), was then added over 2 minutes with gentle agitation. Finally preheated IDMEM (8 ml per 10⁶ B-cells) was added over 3 minutes.

The fused cells were spun down 400×g 6 min and resuspended in 20 ml selection media DMEM (Invitrogen), 15% FBS (Hyclone), supplemented with L-glutamine, pen/strep, MEM Non-essential amino acids, Sodium Pyruvate, 2-Mercaptoethanol (all from Invitrogen), HA-Azaserine Hypoxanthine and OPI (oxaloacetate, pyruvate, bovine insulin) (both from Sigma) and IL-6 (Boehringer Mannheim)) per million B-cells. Cells were incubated for 20-30 min at 37 C and then resuspended in 200 ml selection media and cultured for 3-4 days in T175 flask prior to 96 well plating. Accordingly, hybridomas that produced antigen binding proteins to PCSK9 were produced.

### Example 3

### Selection of PCSK9 Antibodies

The present example outlines how the various PCSK9 antigen binding proteins were characterized and selected. The binding of secreted antibodies (produced from the hybridomas produced in Examples 1 and 2) to PCSK9 was assessed. Selection of antibodies was based on one or more of the following characteristics; binding data, inhibition of PCSK9 binding to LDLR, pH sensitive binding, domain-specific binding and affinity.

### Primary Screen

A primary screen for antibodies which bind to wild-type PCSK9 was performed. The primary screen was performed on two harvests. The primary screen comprised an ELISA assay and was performed using the following protocol:

Costar 3702 medium binding 384 well plates (Corning Life Sciences) were employed. The plates were coated with neutravadin at a concentration of 4 μg/ml in 1×PBS/0.05% Azide, at a volume of 40 μl/well. The plates were incubated at 4° C. overnight. The plates were then washed using a Titertek plate washer (Titertek, Huntsville, Ala.). A 3-cycle wash was performed. The plates were blocked with 90 μl of 1×PBS/1% milk and incubated approximately 30 minutes at room temperature. The plates were then washed. Again, a 3-cycle wash was performed. The capture sample was biotinylated-PCSK9, and was added at 0.9 μg/ml in 1×PBS/1% milk/10 mM Ca²⁺ at a volume of 40 μl/well. The plates were then incubated for 1 hour at room temperature. Next, the plates were washed using the Titertek plate washer operated using a 3-cycle wash. 10 μl of supernatant was transferred into 40 μl of 1×PBS/1% milk/10 mM Ca²⁺ and incubated 1.5 hours at room temperature. Again the plates were washed using the Titertek plate washer operated using a 3-cycle wash. 40 μl/well of Goat anti-Human IgG Fc HRP at a concentration of 100 ng/ml (1:4000) in 1λ PBS/1% milk/10 mM Ca²⁺ was added to the plate and was incubated 1 hour at room temperature. The plates were washed once again, using a 3-cycle wash. Finally, 40 μl/well of One-step TMB (Neogen, Lexington, Ky.) was added to the plate and quenching with 40 μl/well of 1N hydrochloric acid was performed after 30 minutes at room temperature. OD's were read immediately at 450 nm using a Titertek plate reader. Results are reported in Table 10A below. Positive binding was determined as 3 times the average of signal of the negative control which was hybridoma supernatant. The average OD for the negative control was 0.17.

TABLE 10A

| | Early | Late |
|---|---|---|
| | primary | primary |
| | screen | screen |
| Plate | OD | OD |
| 101F7 | 3.45 | 3.91 |
| 103B7 | 4.03 | 3.46 |
| 131D12 | 3.30 | 3.56 |
| 27B2 | 4.90 | 4.20 |

Multiple rounds of Immunization, hybridoma generation and primary screening resulted in the identification of 830 antigen specific hybridomas being identified. The panels were then advanced to screening for the ability to block the LDLR binding interaction.

### Large Scale Receptor Ligand Blocking Screen

To screen for the antibodies that block PCSK9 binding to LDLR an assay was developed using the D374Y PCSK9 mutant. The mutant was used for this assay because it has a higher binding affinity to LDLR allowing a more sensitive receptor ligand blocking assay to be developed. The following protocol was employed in the receptor ligand blocking screen: Costar 3702 medium binding 384 well plates (Corning Life Sciences) were employed in the screen. The plates were coated with goat anti-LDLR (R&D Cat #AF2148) at 2 μg/ml in 1λPBS/0.05% Azide at a volume of 40 μl/well. The plates were incubated at 4° C. overnight. The plates were then washed using a Titertek plate washer (Titertek, Huntsville, Ala.). A 3-cycle wash was performed. The plates were blocked with 90 μl of 1λ PBS/1% milk and incubated approximately 30 minutes at room temperature. The plates were then washed using the Titertek plate washer. A 3-cycle wash was performed. The capture sample was LDLR (R&D, Cat #2148LD/CF), and was added at 0.4 μg/ml in 1×PBS/1% milk/10 mM Ca²⁺ at a volume of 40 μl/well. The plates were then incubated for 1 hour and 10 minutes at room temperature. Contemporaneously, 20 ng/ml of biotinylated human D374Y PCSK9 was incubated with 15 microliters of hybridoma exhaust supernatant in Nunc polypropylene plates and the exhaust supernatant concentration was diluted 1:5. The plates were then pre-incubated for about 1 hour and 30 minutes at room temperature. Next, the plates were washed using the Titertek plate washer operated using a 3-cycle wash. 50 μl/well of the pre-incubated mixture was transferred onto the LDLR coated ELISA plates and incubated for 1 hour at room temperature. To detect LDLR-bound b-PCSK9, 40 μl/well streptavidin HRP at 500 ng/ml in assay diluent was added to the plates. The plates were incubated for 1 hour at room temperature. The plates were again washed using a Titertek plate washer. A 3-cycle wash was performed. Finally, 40 μl/well of One-step TMB (Neogen, Lexington, Ky.) was added to the plate and was quenched with 40 μl/well of 1N hydrochloric acid after 30 minutes at room temperature. OD's were read immediately at 450 nm using a Titertek plate reader. The screen identified 384 antibodies that blocked the interaction between PCSK9 and the LDLR well, 100 antibodies blocked the interaction strongly (OD<0.3). These antibodies inhibited the binding interaction of PCSK9 and LDLR greater than 90% (greater than 90% inhibition). Results are reported in Table 3D below.

TABLE 10D

| | % R-L |
|---|---|
| Plate | Inhibition |
| 101F7 | 94% |
| 103B7 | 93% |
| 131D12 | 92% |
| 27B2 | 93% |

### Example 4

### Antibody Competition Binning by ELISA

Hybridoma supernatants were then assayed for direct binding competition. To screen for antibody competition an ELISA based assay was developed using the following protocol: Non-treated 384 well microtiter plates from Corning Costar Catalogue number 3702, were coated with the following antibodies using 40 ul/well in 1λ PBS with 0.01% sodium azide: 31H4 at 1.0 ug/ml, 26H5 at 1.0 ug/ml, Plates were wrapped in plastic stored at ambient room temperature overnight. The next day each hybridoma supernatant was preincubated with 100 ng/ml b-PCSK9 at a 1:5 final dilution. These samples were then incubated for 2 hours at RT. The precoated antibody capture plates were then washed and the pre-incubated samples of hybridoma Antibody supernatants and B-PCSK9 were added to the plates and incubated for 1 hour at room temperature. Detection of bound b-PCSK9 was done with streptavidin HRP (Thermo P21126) at 500 ng/ml in 1×PBS/1% non fat skim milk. Incubated for 1 hour then added chromogenic substrate TMB, 3,3′,5,5′-tetramethylbenzidine, 40 ul/well incubated anywhere from 15 minutes to 60 minutes depending on OD then stopped with one molar hydrochloric acid. Optical density at 450 nm read on Multiskan Ascent reader. A % Inhibition was then calculated by comparing the amount of bound b-PCSK9 after preincubation with anti-PCSK9 hybridoma sups versus negative control wells using irrelevant hybridoma supernatants. % Inhibition=1−(OD of Ab supernatant/B-PCSK9/OD of Neg. control supernatant/B-PCSK9). The results are presented in Table 11 below.

TABLE 11A

| | % | % |
|---|---|---|
| | inhibition | inhibition |
| | of 31H4 | of 26H5 |
| Plate | Binding | Binding |
| 101F7 | 82% | 92% |
| 103B7 | 79% | 90% |
| 131D12 | 89% | 90% |

Antibodies 31H4 and 26H5 have the following amino acid sequences:

TABLE 11B

| Contained | | SEQ ID | |
|---|---|---|---|
| in Clone | Designation | NO. | Amino Acid Sequence |
| 31H4 | Heavy | 73 | EVQLVESGGGLVKPGGSLRLSCAASGFTFSSYS |
| | Chain | | MNWVRQAPGKGLEWVSSISSSSSYISYADSVKG |
| | | | RFTISRDNAKNSLYLQMNSLRAEDTAVYFCARD |
| | | | YDFWSAYYDAFDVWGQGTMVTVSS |
| | | | ASTKGPSVFPLAPCSRSTSESTAALGCLVKDYFP |
| | | | EPVTVSWNSGALTSGVHTF |
| | | | PAVLQSSGLYSLSSVVTVPSSNFGTQTYTCNVD |
| | | | HKPSNTKVDKTVERKCC |
| | | | VECPPCPAPPVAGPSVFLFPPKPKDTLMISRTPE |
| | | | VTCVVVDVSHEDPEVQ |
| | | | FNWYVDGVEVHNAKTKPREEQFNSTFRVVSVL |
| | | | TVVHQDWLNGKEYKCKVS |
| | | | NKGLPAPIEKTISKTKGQPREPQVYTLPPSREEM |
| | | | TKNQVSLTCLVKGFYP |
| | | | SDIAVEWESNGQPENNYKTTPPMLDSDGSFFLY |
| | | | SKLTVDKSRWQQGNVFS |
| | | | CSVMHEALHNHYTQKSLSLSPGK |
| 8A3 | Light Chain | 74 | QSVLTQPPSVSGAPGQRVTISCTGSSSNIGAGYD |
| 31H4 | | | VHWYQQLPGTAPKLLISGNSNRPSGVPDRFSGS |
| | | | KSGTSASLAITGLQAEDEADYYCQSYDSSLSGS |
| | | | VFGGGTKLTVL |
| | | | QPKANPTVTLFPPSSEELQANKATLVCLISDFYP |
| | | | GAVTV |
| | | | AWKADGSPVKAGVETTKPSKQSNNKYAASSYL |
| | | | SLTPEQWKSHRSYSCQVT |
| | | | HEGSTVEKTVAPTECS |
| 26H5 | Heavy | 75 | QVQLVQSGAEVKKPGASVKVSCKASGYTLTSY |
| | Chain | | GISWVRQAPGQGLEWMGWISFYNGNTNYAQK |
| | | | VQGRVTMTTDTSTSTVYMELRSLRSDDTAVYY |
| | | | CARGYGMDVWGQGTTVTVSS |
| | | | ASTKGPSVFPLAPCSRSTSESTAALGCLVKDYFP |
| | | | EPVTVSWNSGALTSGVHTF |
| | | | PAVLQSSGLYSLSSVVTVPSSNFGTQTYTCNVD |
| | | | HKPSNTKVDKTVERKCC |
| | | | VECPPCPAPPVAGPSVFLFPPKPKDTLMISRTPE |
| | | | VTCVVVDVSHEDPEVQ |
| | | | FNWYVDGVEVHNAKTKPREEQFNSTFRVVSVL |
| | | | TVVHQDWLNGKEYKCKVS |
| | | | NKGLPAPIEKTISKTKGQPREPQVYTLPPSREEM |
| | | | TKNQVSLTCLVKGFYP |
| | | | SDIAVEWESNGQPENNYKTTPPMLDSDGSFFLY |
| | | | SKLTVDKSRWQQGNVFS |
| | | | CSVMHEALHNHYTQKSLSLSPGK |
| 26H5 | Light Chain | 76 | QSALTQPASVSGSPGQSITISCTGTSSDVGGYNS |
| | | | VSWYQQHPGKPPKLMIYEVSNRPSGVSIRFSGS |
| | | | KSGNTASLTISGLQAEDEADYFCSSYTSTSMVF |
| | | | GGGTKLTVL |
| | | | QPKANPTVTLFPPSSEELQANKATLVCLISDFYP |
| | | | GAVTV |
| | | | AWKADGSPVKAGVETTKPSKQSNNKYAASSYL |
| | | | SLTPEQWKSHRSYSCQVT |
| | | | HEGSTVEKTVAPTECS |

### Example 5

### Sequence Analysis of Antibody Heavy and Light Chains

The nucleic acid and amino acid sequences for the light and heavy chains of the above antibodies were then determined by Sanger (dideoxy) nucleotide sequencing. Amino acid sequences were then deduced for the nucleic acid sequences. The sequences of 101F7, 103B7, 131D12 and 27B2.6 are presented in Tables 1-6, herein.

### Example 6

### Binding Affinity Characterization of Antibodies

Binding of anti-PCSK9 antibodies to human and cynomolgus monkey PCSK9 was measured by solution equilibrium binding assay on BIAcore. Antibody was immobilized on the second flow cell of a CM5 chip using amine coupling (reagents provided by GE Healthcare, Piscataway, N.J.) with an approximate density of 6000 RU. The first flow cell was used as a background control. For assay at pH 7.4, 0.1 nM or 1.0 nM of PCSK9 were mixed with serial dilutions of antibody (ranging from 0.003 nM to 50 nM) in PBS plus 0.1 mg/mL BSA, 0.005% P20 and incubated at room temperature for 4 hours. For assay at pH 5.5, 0.1 nM or 1.0 nM of PCSK9 were mixed with serial dilutions of antibody (ranging from 0.003 nM to 50 nM) in 10 mM Sodium Citrate, pH 5.5, plus 150 mM NaCl, 0.1 mg/mL BSA, 0.005% P20 and incubated at room temperature for 4 hours. Binding of free PCSK9 in the mixed solutions was measured by injecting over the antibody coated chip surface. One hundred percent PCSK9 binding signal on the antibody surface was determined in the absence of antibody in the solution. A decreased PCSK9 binding response with increasing concentrations of antibody in solution indicated PCSK9 was binding to the antibody, preventing PCSK9 from binding to the immobilized antibody surface. Plotting the PCSK9 binding signal versus antibody concentration, the KD was obtained from nonlinear regression of the competition curves using an n-curve one-site homogeneous binding model provided in the KinExA Pro software. The results are presented in Table 12 below:

TABLE 12
Analysis of the binding of antibodies to human and cynomolgus PCSK9 by
solution based equilibrium assay

| | huPCSK9-pH 7.4 | | huPCSK9-pH 5.5 | | cyPCSK9-pH 7.4 | | cyPCSK9-pH 5.5 | |
|---|---|---|---|---|---|---|---|---|
| | KD (pM) | 95% CI (pM) | KD (pM) | 95% CI (pM) | KD (pM) | 95% CI (pM) | KD (pM) | 95% CI (pM) |
| 101F7 | 6 | 4~7 | 16 | 13~20 | 8 | 7~9 | 8 | 6~9 |
| 103B7 | 7 | 5~8 | 18 | 14~23 | 11 | 8~14 | 22 | 20~26 |
| 131D12 | 67 | 47~92 | 120 | 94~160 | 1500 | 1200~1800 | 3100 | 2500~3700 |

1. An isolated anti-PCSK9 antigen binding protein, comprising an immunoglobulin heavy chain polypeptide, or functional fragment thereof, and an immunoglobulin light chain polypeptide, or functional fragment thereof, wherein the anti-PCSK9 antigen binding protein competes for binding to human PCSK9 with an 101F7, 103B7 or 131D12 antigen binding protein.

2. The isolated anti-PCSK9 antigen binding protein of claim 1 that competes for binding to human PCSK9 with both antibody 31H4 and antibody 26H5.

3. The isolated anti-PCSK9 antigen binding protein of claim 1 comprising an immunoglobulin heavy chain variable domain polypeptide, or functional fragment thereof having at least 85% sequence identity with any one of SEQ ID NO: 20, 21, 22, or 23.

4. The isolated anti-PCSK9 antigen binding protein of claim 3 comprising an immunoglobulin heavy chain variable domain polypeptide, or functional fragment thereof having at least 90% sequence identity with any one of SEQ ID NO: 20, 21, 22, or 23.

5. The isolated anti-PCSK9 antigen binding protein of claim 4 comprising an immunoglobulin heavy chain variable domain polypeptide, or functional fragment thereof having at least 95% sequence identity with any one of SEQ ID NO: 20, 21, 22, or 23.

6. The isolated anti-PCSK9 antigen binding protein of claim 5 comprising an immunoglobulin heavy chain variable domain polypeptide, or functional fragment thereof having the amino acid sequence according to any one of SEQ ID NO: 20, 21, 22, or 23.

7. The isolated anti-PCSK9 antigen binding protein of claim 1 comprising an immunoglobulin light chain variable domain polypeptide, or functional fragment thereof having at least 85% sequence identity with any one of SEQ ID NO: 16, 17, 18 or 19.

8. The isolated anti-PCSK9 antigen binding protein of claim 7 comprising an immunoglobulin light chain variable domain polypeptide, or functional fragment thereof having at least 90% sequence identity with any one of SEQ ID NO: 16, 17, 18 or 19.

9. The isolated anti-PCSK9 antigen binding protein of claim 8 comprising an immunoglobulin light chain variable domain polypeptide, or functional fragment thereof having at least 95% sequence identity with any one of SEQ ID NO: 16, 17, 18 or 19.

10. The isolated anti-PCSK9 antigen binding protein of claim 9 comprising an immunoglobulin light chain variable domain polypeptide, or functional fragment thereof having the amino acid sequence according to any one of SEQ ID NO: 16, 17, 18 or 19.

11. The anti-PCSK9 antigen binding protein of claim 1, wherein the antigen binding protein is a monoclonal antibody.

12. The anti-PCSK9 antibody of claim 11, wherein the antibody is humanized.

13. The anti-PCSK9 antibody of claim 11, wherein the antibody is human.

14. The anti-PCSK9 antibody of any of claim 11, wherein the antibody is an antibody fragment selected from a Fab, Fab′-SH, Fv, scFv or (Fab′).sub.2 fragment.

15. The anti-PCSK9 antibody of claim 11, wherein at least a portion of the framework sequence is a human consensus framework sequence.

16. An isolated nucleic acid encoding an anti-PCSK9 antigen binding protein of claim 1.

17. A vector comprising the nucleic acid of claim 16.

18. The vector of claim 17, wherein the vector is an expression vector.

19. A host cell comprising the vector of claim 17.

20. The host cell of claim 19, wherein the host cell is prokaryotic.

21. The host cell of claim 19, wherein the host cell is eukaryotic.

22. A method for making an anti-PCSK9 antigen binding protein, said method comprising culturing a host cell comprising a vector comprising a nucleic acid encoding the anti-PCSK9 antigen binding protein of claim 1 under conditions suitable for expression of the nucleic acid encoding the anti-PCSK9 antibody.

23. The method of claim 22, further comprising recovering the anti-PCSK9 antigen binding protein from the host cell.

24. A pharmaceutical composition comprising the anti-PCSK9 antigen binding protein of claim 1 and a pharmaceutically acceptable carrier.

25. A method of reducing LDL-cholesterol level in a subject, said method comprising administering to the subject an effective amount of the anti-PCSK9 antigen binding protein of claim 1.

26. A method of treating cholesterol related disorder in a subject, said method comprising administering to the subject an effective amount of the anti-PCSK9 antigen binding protein of claim 1.

27. A method of treating hypercholesterolemia in a subject, said method comprising administering to the subject an effective amount of the anti-PCSK9 antigen binding protein of claim 1.

28. The method of claim 25, further comprising administering to the subject an effective amount of a second medicament, wherein the anti-PCSK9 antigen binding protein is the first medicament.

29. The method of claim 28, wherein the second medicament elevates the level of LDLR.

30. The method of claim 28, wherein the second medicament reduces the level of LDL-cholesterol.

31. The method of claim 28, wherein the second medicament comprises a statin.

32. The method of claim 31, wherein the statin is selected from the group consisting of atorvastatin, fluvastatin, lovastatin, mevastatin, pitavastatin, pravastatin, rosuvastatin, simvastatin, and any combination thereof.

33. A method of inhibiting binding of PCSK9 to LDLR in a subject, said method comprising administering to the subject an effective amount of the anti-PCSK9 antigen binding protein of claim 1.

34. A method of detecting PCSK9 protein in a sample, said method comprising (a) contacting the sample with the antigen binding protein of claim 1; and (b) detecting formation of a complex between the anti-PCSK9 antigen binding protein and the PCSK9 protein.

35. The isolated anti-PCSK9 antigen binding protein of claim 3 comprising an immunoglobulin light chain variable domain polypeptide, or functional fragment thereof having at least 85% sequence identity with any one of SEQ ID NO: 16, 17, 18 or 19.

36. The isolated anti-PCSK9 antigen binding protein of claim 4 comprising an immunoglobulin light chain variable domain polypeptide, or functional fragment thereof having at least 90% sequence identity with any one of SEQ ID NO: 16, 17, 18 or 19.

37. The isolated anti-PCSK9 antigen binding protein of claim 5 comprising an immunoglobulin light chain variable domain polypeptide, or functional fragment thereof having at least 95% sequence identity with any one of SEQ ID NO: 16, 17, 18 or 19.

38. The isolated anti-PCSK9 antigen binding protein of claim 6 comprising an immunoglobulin light chain variable domain polypeptide, or functional fragment thereof having the amino acid sequence according to any one of SEQ ID NO: 16, 17, 18 or 19.
